(19) United States
(12) Patent Application Publication
(10) Pub. No.: US 2024/0382473 A1
(43) Pub. Date: Nov. 21, 2024

(54) Combination Therapies for Treatment of Myelodysplastic Syndrome

(71) Applicant: Sumitomo Pharma Oncology, Inc., Marborough, MA (US)

(72) Inventors: Steven L. Warner, Sandy, UT (US); Stephen Patrick Anthony, Heber City, UT (US); David J. Bearss, Alpine, UT (US); Michael Vincent McCullar, Walnut Creek, CA (US)

(21) Appl. No.: 17/782,033

(22) PCT Filed: Dec. 4, 2020
(86) PCT No.: PCT/US2020/063381

§ 371 Date: Jun. 2, 2022

Related U.S. Application Data
(60) Provisional application No. 63/037,553, filed on Jun. 10, 2020.
(60) Provisional application No. 62/944,339, filed on Dec. 5, 2019.

(51) Int. Cl. A61K 31/453; A61K 31/675; A61K 31/706; A61P 35/02

(52) U.S. Cl. CPC A61K 31/453; A61K 31/675; A61K 31/706; A61P 35/02

(57) ABSTRACT

The present invention relates to methods for treatment of myelodysplastic syndrome (MDS) by administration of a hypomethylating agent (HMA), such as azacitidine or decitabine, or a prodrug of either of the foregoing, or a pharmaceutically acceptable salt of any of the foregoing, and alvocidib, or a prodrug thereof, or a pharmaceutically acceptable salt of the foregoing.

### RELATED APPLICATIONS

This application is the U.S. National Stage of International Application No. PCT/US2020/063381, filed on Dec. 4, 2020, published in English, which claims the benefit of U.S. Application No. 63/037,553, filed on Jun. 10, 2020, and U.S. Application No. 62/944,339, filed on Dec. 5, 2019. The entire teachings of the above applications are incorporated herein by reference.

### FIELD OF INVENTION

The present invention relates to methods for treatment of myelodysplastic syndrome (MDS) by administration of a hypomethylating agent (HMA) and alvocidib.

### BACKGROUND OF THE INVENTION

Myelodysplastic Syndrome (MDS) is a diverse group of bone marrow disorders characterized by the inability to produce healthy numbers of blood cells. Frequently (in around 30% of cases), MDS progresses to acute myelogenous leukemia (AML), which remains largely an incurable condition with relatively poor survival rates. One treatment option for MDS is the hypomethylating agents (HMAs) azacitidine and deoxyazacitidine (decitabine), which act by two principal mechanisms: 1) by inhibiting DNA methyltransferases, which leads to the activation of key tumor suppressor genes; and 2) by directly damaging DNA following incorporation into replicating DNA strands. The second of these mechanisms activates the programmed cell-death pathway (apoptosis), and this pathway has been shown to depend somewhat on the expression levels of key apoptosis regulatory proteins, including MCL-1, an anti-apoptotic member of the BH3 family of apoptotic regulating proteins.

Cyclin-dependent kinases (CDKs) are important regulators that control the timing and coordination of the cell cycle. CDKs form reversible complexes with their obligate cyclin partners to control transition through key junctures in the cell cycle. In addition to regulating cell cycle progression, some CDK family members, for example, CDK7 and CDK9, also play an active role in transcription. In particular, CDK9 directly phosphorylates RNA polymerase II and contributes to productive transcription. Agents which inhibit CDK9 have been shown to inhibit the expression of MCL-1, an important protein in the apoptosis pathway activated by DNA methyltransferase inhibitors. One such CDK inhibitor is alvocidib, which is a potent and selective inhibitor of the CDKs (e.g., CDK-9) and has antitumor activity against various tumor cell lines. Alvocidib is also known to rapidly decrease expression levels of MCL-1.

While progress has been made with combinations of DNA methyltransferase inhibitors and cyclin-dependent kinase inhibitors for the treatment of cancers, there remains a need in the art for improved combination therapies and improved methods for treatment of MDS. The present invention fulfills this need and provides related advantages.

### SUMMARY OF THE INVENTION

Various non-limiting aspects and embodiments of the invention are described below.

In one aspect, the present disclosure provides a method of treating myelodysplastic syndrome (MDS) in a patient in need thereof, comprising administering to the patient a therapeutically effective amount of a hypomethylating agent (HMA) (e.g., azacitidine, or a prodrug thereof, or a pharmaceutically acceptable salt of the foregoing; or decitabine, or a prodrug thereof, or a pharmaceutically acceptable salt of the foregoing) and a therapeutically effective amount of alvocidib, or a prodrug thereof, or a pharmaceutically acceptable salt of the foregoing, wherein the patient has previously untreated MDS; received fewer than six cycles of treatment with a hypomethylating agent; de novo MDS and/or secondary MDS.

In another aspect, the present disclosure provides a method of treating MDS in a patient in need thereof, comprising administering to the patient a therapeutically effective amount of azacitidine, or a prodrug thereof, or a pharmaceutically acceptable salt of the foregoing; and a therapeutically effective amount of alvocidib, or a prodrug thereof, or a pharmaceutically acceptable salt of the foregoing. The azacitidine, or a prodrug thereof, or a pharmaceutically acceptable salt of the foregoing, is administered on days 1, 2, 3, 4, 5, 6 and 7, or on days 1, 2, 3, 4, 5, 8 and 9 of a 28-day treatment cycle. The alvocidib, or a prodrug thereof, or a pharmaceutically acceptable salt of the foregoing, is administered on day 10 of the 28-day treatment cycle.

In yet another aspect, the present disclosure provides a method of treating MDS in a patient in need thereof, comprising administering to the patient a therapeutically effective amount of decitabine, or a prodrug thereof, or a pharmaceutically acceptable salt of the foregoing; and a therapeutically effective amount of alvocidib, or a prodrug thereof, or a pharmaceutically acceptable salt of the foregoing. The decitabine, or a prodrug thereof, or a pharmaceutically acceptable salt of the foregoing, is administered on days 1, 2, 3, 4 and 5 of a 28-day treatment cycle. The alvocidib, or a prodrug thereof, or a pharmaceutically acceptable salt of the foregoing, is administered on day 8 of the 28-day treatment cycle.

In another aspect, the present disclosure provides a method of treating myelodysplastic syndrome (MDS) in a patient with previously untreated MDS comprising administering to the patient a therapeutically effective amount of a hypomethylating agent (HMA) and a therapeutically effective amount of alvocidib.

In another aspect, the present disclosure provides a method of treating MDS in a patient who received fewer than six cycles of treatment with a hypomethylating agent (HMA) comprising administering to the patient a therapeutically effective amount of an HMA and a therapeutically effective amount of alvocidib.

In another aspect, the present disclosure provides a method of treating MDS in a patient with de novo MDS who has not received a previous MDS treatment comprising administering to the patient a therapeutically effective amount of a hypomethylating agent (HMA) and a therapeutically effective amount of alvocidib.

In one embodiment, the patient is not eligible for intensive induction chemotherapy or a stem cell transplant.

In one embodiment, the patient has one or more mutations in one or more of RUNX1, SRSF2, SF3B1, U2AF1, ZRSR2, ASXL1, EZH2, BCOR and STAG2.

In another aspect, the present disclosure provides a method of treating MDS in a patient with secondary MDS who has not received a previous MDS treatment comprising administering to the patient a therapeutically effective amount of a hypomethylating agent (HMA) and a therapeutically effective amount of alvocidib.

In one embodiment, the patient is not eligible for intensive induction chemotherapy or a stem cell transplant.

In one embodiment, the patient has one or more mutations in one or more of RUNX1, SRSF2, SF3B1, U2AF1, ZRSR2, ASXL1, EZH2, BCOR and STAG2.

In another embodiment, the MDS is selected from the group consisting of refractory anemia (RA), refractory anemia with ringed sideroblasts (RARS), refractory anemia with excess blasts (RAEB), refractory anemia with excess blasts in transformation (RAEB-T), and chronic myelomonocytic leukemia (CMML).

In another embodiment, the MDS is selected from an intermediate-1 Revised International Prognostic Scoring System (IPSS-R) group, an intermediate-2 IPSS-R group, and a high IPSS-R group.

In another embodiment, the HMA and the alvocidib are administered simultaneously.

In another embodiment, the HMA and the alvocidib are administered sequentially.

In another embodiment, the HMA is administered first, followed by administration of alvocidib.

In another embodiment, the alvocidib is administered during a period of elevated NOXA expression following HMA administration.

In another embodiment, the HMA is administered as a prodrug.

In another embodiment, the alvocidib is administered as a prodrug.

In another embodiment, the alvocidib prodrug is an alvocidib phosphate prodrug.

In another embodiment, the alvocidib phosphate prodrug is a compound having the structure

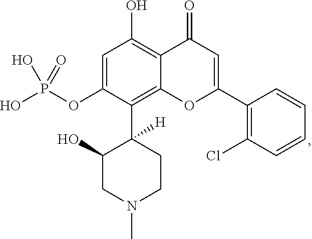 [SMILES: CN1CC[C@H](c2c(OP(=O)(O)O)cc(O)c3c(=O)cc(-c4ccccc4Cl)oc23)[C@H](O)C1]

or a pharmaceutically acceptable salt thereof.

In another embodiment, the HMA is administered in combination with a cytidine deaminase inhibitor.

In another embodiment, the HMA is administered intravenously or by subcutaneous injection.

In another embodiment, the HMA is selected from azacitidine and decitabine.

In another embodiment, the HMA is azacitidine.

In another embodiment, the azacitidine is administered as an azacitidine phosphate prodrug.

In another embodiment, the azacitidine phosphate prodrug has the formula

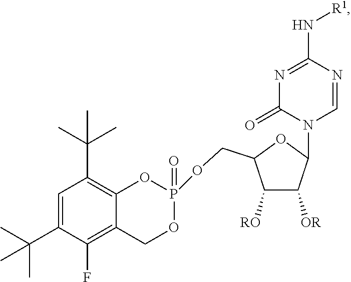

where R and R¹ are independently H or CO₂(C₁-C₆ alkyl).

In another embodiment, R is H at each occurrence and R1 is selected from H and CO₂(C₅ alkyl).

In another embodiment, the azacitidine is 2′,3′,5′-triacetyl-5-azacitidine.

In another embodiment, the azacitidine is administered orally.

In another embodiment, the azacitidine is administered as CC-486 composition.

In another embodiment, the azacitidine is administered as an intravenous infusion.

In another embodiment, the intravenous infusion is over from about 5 to about 100 minutes.

In another embodiment, the intravenous infusion is over from about 10 to about 40 minutes.

In another embodiment, the azacitidine is administered subcutaneously.

In another embodiment, the azacitidine is administered consecutively for 7 days.

In another embodiment, the azacitidine is administered once daily for 5 days, followed by 2 azacitidine-free days, then followed by once daily administration of azacitidine for 2 days.

In another embodiment, the azacitidine is administered at a dosage of about 10 mg/m² to about 90 mg/m².

In another embodiment, the azacitidine is administered at a dosage lower than about 90 mg/m² and subsequently escalated to the dosage of about 90 mg/m².

In another embodiment, the azacitidine is administered at a dosage of about 75 mg/m².

In another embodiment, the alvocidib is administered on day 10 from the start of the azacitidine administration.

In another embodiment, the alvocidib is administered during a period of elevated NOXA expression following azacitidine administration.

In another embodiment, the alvocidib is administered as an intravenous infusion.

In another embodiment, the intravenous infusion is over from about 20 to about 120 minutes.

In another embodiment, the intravenous infusion over about 1 hour.

In another embodiment, the alvocidib is administered at a dosage of about 90 mg/m².

In another embodiment, the HMA is decitabine.

In another embodiment, the decitabine is administered in combination with cedazuridine.

In another embodiment, the decitabine is administered as an intravenous infusion.

In another embodiment, the intravenous infusion is over from about 20 to about 120 minutes.

In another embodiment, the intravenous infusion over about 1 hour.

In another embodiment, the decitabine is administered daily for 5 days.

In another embodiment, the alvocidib is administered on day 8 from the start of the decitabine administration.

In another embodiment, the alvocidib is administered during a period of elevated NOXA expression following decitabine administration.

In another embodiment, the alvocidib is administered as a bolus followed by an intravenous infusion.

In another embodiment, the bolus is over about 10 to about 40 minutes.

In another embodiment, the bolus is over about 30 minutes.

In another embodiment, the intravenous infusion is over from about 30 minutes to about 6 hours.

In another embodiment, the intravenous infusion is over about 4 hours.

In another embodiment, the alvocidib is administered as a bolus at a dosage of about 20 mg/m² followed by an intravenous infusion at a dosage of about 10 mg/m² to about 60 mg/m².

In another embodiment, the alvocidib is administered at an overall dosage of about 20 mg/m² to about 100 mg/m².

In another embodiment, the alvocidib is administered as an intravenous infusion.

In another embodiment, the intravenous infusion is over about 1 hour.

In another embodiment, the alvocidib is administered at a dosage of about 90 mg/m².

In another embodiment, the decitabine is administered at a daily dosage of about 10 mg/m² to about 30 mg/m².

In another embodiment, the decitabine is administered at a daily dosage of about 20 mg/m².

In another embodiment, the patient is further administered a tumor lysis syndrome prophylaxis.

In another embodiment, the tumor lysis syndrome prophylaxis comprises intravenous hydration with a 0.45% aqueous NaCl.

In another embodiment, the tumor lysis syndrome prophylaxis comprises administering one or more of allopurinol, an oral phosphate binder, replacement of fluid losses, and an anti-diarrheal medication.

In another embodiment, the tumor lysis syndrome prophylaxis is administered prior to first HMA dose.

In another embodiment, the tumor lysis syndrome prophylaxis is administered prior to first alvocidib dose.

In another embodiment, the patient is 18 years old or greater.

In another embodiment, the patient has an Eastern Cooperative Oncology Group (ECOG) Performance Status (PS) score which is less than or equal to 2.

In another embodiment, the patient has a life expectancy of greater than or equal to 3 months.

In another embodiment, the patient has one or more mutations in one or more of RUNX1, SRSF2, SF3B1, U2AF1, ZRSR2, ASXL1, EZH2, BCOR and STAG2.

In another embodiment, the patient meets the following criteria based on laboratory data:
a) wherein the patient's serum creatinine is less than or equal to 1.8 times the upper limit of the normal (ULN) range;
b) wherein the patient's total bilirubin is less than or equal to 2 times the ULN range, and
c) wherein the patient's aspartate transaminase (AST) and alanine transaminase (ALT) are less than or equal to 3 times the ULN range.

In another embodiment, the patient does not have a concomitant severe cardiovascular disease.

In another embodiment, the patient does not have a condition selected from New York Heart Association (NYHA) Functional Class III or IV heart disease, National Cancer Institute (NCI) Common Terminology Criteria for Adverse Events (CTCAE) v5.0 grade equal to or greater than 3 arrhythmia, angina pectoris, abnormal electrocardiogram findings, interstitial pneumonia, and pulmonary fibrosis.

In another embodiment, the patient has not had myocardial infarction within 6 months before the treatment.

In another embodiment, the patient does not have a concomitant malignancy requiring chemotherapy, or a concomitant malignancy for which the patient received chemotherapy within 6 months prior to treatment, with the proviso that the malignancy is not selected from basal and squamous cell carcinoma of the skin.

In another embodiment, the patient does not have an uncontrolled or uncontrollable infection, or a Grade equal to or greater than 3 infection according to NCI CTCAE v5.0.

In another embodiment, the patient does not have a dry tap on bone marrow aspiration.

In another embodiment, the patient does not have a concurrent autoimmune disease or a history of chronic or recurrent autoimmune disease.

In another embodiment, the patient does not require a long-term systemic steroid therapy greater than the equivalent of 20 mg of prednisone daily.

In another embodiment, the patient does not have another documented malignancy within the past year.

In another embodiment, the patient does not have Grade equal to or greater than 2 hemorrhage according to NCI CTCAE v5.0.

In another embodiment, the patient is not pregnant or breastfeeding.

In another embodiment, the patient has not previously received alvocidib or another cyclin-dependent kinase 9 (CDK9) inhibitor.

In another embodiment, the method further comprises determining a BH3 profile for the patient's tumor cell specimen.

In another embodiment, the method further comprises measurement of an additional biomarker associated with MDS.

In another embodiment, the additional biomarker associated with MDS is selected from the group consisting of nucleic acids, proteins, lipids, and metabolites.

In another embodiment, the additional biomarker associated with MDS is MCL-1.

In another embodiment, the method further comprises classifying the patient for likelihood of response to MDS treatment based on the patient's BH3 profile.

In another embodiment, the BH3 profile is determined by flow cytometry.

In another aspect, the present disclosure provides a method for determining a response to MDS treatment comprising administering a hypomethylating agent and alvocidib to a patient with previously untreated MDS, the method comprising determining a BH3 profile for the patient's tumor cell specimen, and classifying the patient for likelihood of response to MDS treatment.

In one embodiment, the patient has one or more mutations in one or more of RUNX1, SRSF2, SF3B1, U2AF1, ZRSR2, ASXL1, EZH2, BCOR and STAG2.

In another embodiment, the method further comprises measurement of an additional biomarker associated with MDS.

In another embodiment, the additional biomarker is selected from the group consisting of nucleic acids, proteins, lipids, and metabolites.

In another embodiment, the additional biomarker is MCL-1.

In another embodiment, the BH3 profile is determined by flow cytometry.

In yet another aspect, the present disclosure provides a method of treating a patient with myelodysplastic syndrome (MDS) comprising administering to the patient a therapeutically effective amount of a hypomethylating agent (HMA) selected from azacitidine and decitabine and subsequently administering to the patient a therapeutically effective amount of alvocidib.

In another embodiment, the HMA is administered intravenously.

In another embodiment, the HMA is decitabine administered at a dose of about 10 mg/m² to about 30 mg/m² for from about 1 to about 3 hours, once to three times/day.

In another embodiment, the decitabine is administered once/day for 3 to 7 days.

In another embodiment, the decitabine is administered once/day for 5 days.

In another embodiment, the decitabine is administered at a dose of about 20 mg/m² in a one-hour infusion.

In another embodiment, the alvocidib is administered at a rate of about 10 mg/m² to about 120 mg/m².

In another embodiment, the alvocidib is administered two days after the cessation of the decitabine administration.

In another embodiment, the alvocidib is administered two days after the cessation of the decitabine administration.

In another embodiment, a portion of the alvocidib is administered as a bolus dose of from about 10 mg/m² to about 50 mg/m² over a period of about 10 minutes to about 60 minutes.

In another embodiment, the bolus dose is administered over a period of about 30 minutes.

In another embodiment, the bolus dose is from about 20 mg/m² to about 30 mg/m².

In another embodiment, from about 10 mg/m² to about 60 mg/m² of alvocidib is administered intravenously over a period of about 2 hours to about 6 hours.

In another embodiment, the alvocidib is administered over a period of about 4 hours.

In another embodiment, the dose of the alvocidib is from about 20 mg/m² to about 60 mg/m².

In another embodiment, the alvocidib is administered intravenously at a dose of about 90 mg/m² over a period of about 20 minutes to about 120 minutes.

In another embodiment, the alvocidib is administered over a period of about 1 hour.

In another embodiment, the administration of the alvocidib by intravenous infusion is begun within about 30 minutes of the completion of the bolus dose.

In another embodiment, the HMA is azacitidine at a dose of about 30 to about 90 mg/m².

In another embodiment, the dose is about 75 mg/m² per day.

In another embodiment, the azacitidine is administered for seven days as an intravenous bolus injection over about 10 to about 40 minutes or subcutaneous injection.

In another embodiment, the alvocidib is administered intravenously two days after the cessation of azacitidine administration.

In another embodiment, the alvocidib is administered on day 10 after the commencement of azacitidine administration with no azacitidine administration on days 8 and 9.

In another embodiment, 90 mg/m² of the alvocidib is administered intravenously over a period of about 20 minutes to about 120 minutes.

In another embodiment, the alvocidib is administered over a period of about 1 hour.

In another embodiment, the azacitidine is administered at a dose of about 30 to about 90 mg/m²/day for five consecutive days, followed by azacitidine-free days 6 and 7, further followed by intravenous administration of azacitidine at a dose of about 30 to about 90 mg/m² on days 8 and 9, and further followed by intravenous administration of the alvocidib on day 10.

In another embodiment, the azacitidine is administered at a dose of about 75 mg/m²/day by intravenous bolus injection on days 1 to 5 and days 8 and 9, and wherein the alvocidib is administered at a dose of about 90 mg/m² over a period of about one hour by intravenous infusion on day 10.

In another embodiment, the treatment is repeated at least once.

In another embodiment, the treatment is repeated at least once.

In another embodiment, a treatment cycle comprises 28 days.

In another embodiment, the treatment cycle is repeated at least once.

In another embodiment, the treatment is repeated for at least 4 cycles.

In another embodiment, a treatment cycle comprises four to six weeks.

In another embodiment, the treatment is repeated for at least 4 cycles.

In another embodiment, the HMA is administered orally.

In another embodiment, the HMA is administered as a prodrug.

In another embodiment, the HMA is administered in combination with a cytidine deaminase inhibitor.

In another embodiment, the HMA is decitabine.

In another embodiment, the cytidine deaminase inhibitor is cedazuridine.

In another embodiment, the HMA is an azacitidine phosphate prodrug.

In another embodiment, wherein the azacitidine prodrug has the formula

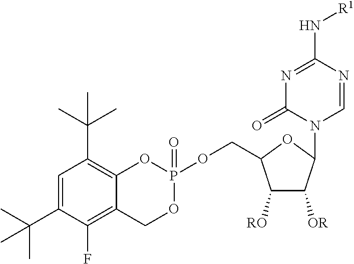

where R and R¹ are independently H or CO₂(C₁-C₆ alkyl).

In another embodiment, the HMA is the composition CC-486.

In another embodiment, the HMA is azacitidine administered as 2′,3′,5′-triacetyl-5-azacitidine.

These and other aspects of the present invention will become apparent to those skilled in the art after a reading of the following detailed description of the invention, including the appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is an illustration providing an overview of CDK functions in a cell.

FIG. 2 is a plot of the percentage of cells undergoing apoptosis (y-axis) following 24 hours alvocidib exposure in nM (x-axis).

FIGS. 3A and 3B are bar graphs showing IC50 values for alvocidib. FIG. 3A is a bar graph of IC50 values (M) for different CDK family members targeted by alvocidib in a cell-free biochemical assay. FIG. 3B is a bar graph of IC50 values (nM) for kinases inhibited by alvocidib at cellular concentrations below 2 [M.

FIG. 4 demonstrates that up-regulation of CDK9 kinase activity and MCL-1 stability contribute to acquired resistance to cyclin-dependent kinase inhibitors in leukemia. The bar graph in the left panel of FIG. 4 shows that cells acquired resistance to flavopiridol-induced cell death in vitro. The top right panel of FIG. 4 shows that phosphorylation of Ser2 of RNA Pol II CTD is more resistant against flavopiridol's drug action. The middle panel on the right of FIG. 4 shows that CDK9 kinase activity is upregulated to promote RNA Pol II activity, counter to the drug mechanism of flavopiridol. The lower right panel of FIG. 4 shows that MCL-1 protein levels are more stable to antagonize the flavopiridol-mediated depletion in Flavo-R.

FIG. 5 is an illustration of MCL-1 transcription regulation by CDK9 and alvocidib disruption of super-enhancer activity via CDK9 inhibition.

FIG. 6 is a diagram showing how CDK9 is part of a super-enhancer complex regulating RNA polymerase II and drives the expression of MCL-1, a member of the BCL-2 family that inhibits apoptosis.

FIGS. 7A and 7B together demonstrate that MCL-1 is regulated by a super-enhancer in AML cells. FIG. 7A is a schematic showing how cooperative interactions of enhancer-associated factors at super-enhancers leads to both higher transcriptional output and increased sensitivity to factor concentration. FIG. 7B shows gene tracks of MED1 (top) and BRD4 (bottom) ChIP-seq occupancy overlapping with and downstream of the MCL1 gene.

FIG. 8 shows that alvocidib inhibits expression of MCL-1. The left panel of FIG. 8 is a Western blot demonstrating a dose-dependent decrease in MCL-1 in response to alvocidib. The right panel of FIG. 8 is a Western blot showing a progressive decrease over time of MCL-1 in MV4-11 cells treated with 80 nM alvocidib. The right panel of FIG. 8 demonstrates that knock-down of MCL-1 exceeds 96 hours.

FIG. 9 is an illustration of a proposed mechanism by which alvocidib inhibits expression of MCL-1 by inhibiting CDK9.

FIG. 10 is a bar graph of normalized MCL-1 gene expression (measured as mRNA) at indicated concentrations of alvocidib.

FIG. 11 shows that NOXA priming (MCL-1 dependence) is associated with alvocidib response. FIG. 11 shows the results of a BH3 profile (mitochondrial profiling) of cells treated with indicated concentrations of alvocidib. Significance values (p-value) are provided.

FIGS. 12A and 12B are diagrams summarizing the design of a Zella 201 clinical trial. FIG. 12A provides an overview of Stage 1 and Stage 2 of the Zella 201 clinical trial.

FIG. 12B provides an overview of a dose and treatment schedule for the Zella 201 clinical trial.

FIG. 13 lists patient characteristics for the Zella 201 clinical trial.

FIG. 14 lists response characteristics for Stage 1 of the Zella 201 clinical trial.

FIG. 15 is a graph summarizing responder (CR/CRi) results form the Zella 201 clinical trial.

FIGS. 16A and 16B show that NOXA priming is predictive of response to alvocidib while NOXA priming is not predictive of response to 7+3 chemotherapy. FIG. 16A is a box-plot showing that high NOXA priming (MCL-1 dependence) is predictive of alvocidib sensitivity in acute myeloid leukemia (AML) patients. FIG. 16B is a box-plot showing that there is no correlation of response to NOXA-primed (MCL-1 dependent) patients to treatment with 7+3 chemotherapy (CR=complete remission; NR=no response).

FIG. 17 is a bar graph demonstrating that MCL-1 dependence is predictive of alvocidib sensitivity in AML patients.

FIGS. 18A and 18B demonstrate that myelodysplastic syndrome (MDS) patients developing AML (secondary AML) are more likely to be responsive to alvocidib treatment than non-MDS AML patients. FIG. 18A is a plot showing that MDS patients are more highly NOXA primed (MCL-1 dependent) than a general AML patient population. Shaded areas represent 95% confidence interval. FIG. 18B is a plot showing that patients with NOXA priming (MCL-1 dependence) greater than or equal to 40% demonstrated greater survival than patients with NOXA priming less than 40%. NOXA priming above 40% is considered highly NOXA primed (or, alternatively, MCL-1 dependent).

FIG. 19 shows that MCL-1 dependency is common in heme malignancies. FIG. 19 graphs the percentage of population estimated to be MCL-1 dependent, as determined by BH3 profiling.

FIG. 20 depicts a hypothesized synergy mechanism between alvocidib and HMAs.

FIGS. 21A and 21B show that alvocidib shows clinical activity in secondary AML. FIG. 21A is a bar graph of CR/CRi rate for treatment groups having secondary AML. FIG. 21B shows results of mitochondrial profiling performed on pre-treatment bone marrow samples (n=24) and correlated with response.

FIGS. 22A and 22C show that alvocidib reduces RNA pol II phosphorylation and MCL-1 expression in vitro. FIGS. 22A and 22B show that alvocidib inhibits RNA pol II phosphorylation in a dose-dependent fashion in MV-4-11 AML cells, as measured by flow cytometry following 24-hour treatment. FIG. 22C is a graphical representation of data shown in FIGS. 22A and 22B.

FIG. 23 is a bar graph showing that phospho-RNA polymerase (pRpb1) and MCL-1 (MCL1) levels are decreased after alvocidib treatment in MDS patient cells.

FIGS. 24A-24C show that HMAs increase NOXA expression in vitro. FIGS. 24A and 24C are Western blots and FIG. 24B is a bar graph of mRNA concentrations. Together, FIGS. 24A-C demonstrate that HMAs, such as decitabine, induce NOXA re-expression.

FIG. 25 is a Western blot showing that azacitidine induces NOXA expression in vitro. MV-4-11 AML cells were treated with indicated concentrations of azacitidine for 24 hours and protein assessed by Western blot for NOXA expression. A dose-dependent increase of NOXA expression was observed.

FIGS. 26A and 26B are scatter plots showing cell viability following 48 hours treatment at indicated concentrations of azacytidine or alvocidib. FIG. 26A is a plot of cell viability following 48-hour treatment against indicated concentrations of azacitidine. IC₅₀ for azacitidine treatment was 1031 nM. FIG. 26B is a plot of cell viability following 48-hour treatment against indicated concentrations of alvocidib. IC₅₀ for azacitidine treatment was 95.63 nM.

FIGS. 27A and 27B demonstrate a synergy between alvocidib and HMAs to induce apoptosis. FIG. 27A is a plot of cell viability in cells treated with DMSO (control) and indicated concentrations of azacytidine or 80 nM alvocibib and indicated concentrations of azacitidine. The lower panel of FIG. 27A lists EC50 values determined from the plot of cell viability. Cell viability was assessed (Celltiter-glo) in MV4-11 cells. FIG. 27B is a bar graph of caspase activity in cells treated with DMSO (control), decitabine, alvocidib, or decitabine and alvocidib.

FIGS. 28A and 28B show that alvocidib inhibits upregulation of MCL-1 by azacytidine without affecting NOXA induction in MV-4-11 cells. FIG. 28A is an overview of an experimental used to evaluate an interaction between alvocidib and azacytidine with regard to MCL-1 and NOXA expression. FIG. 28B is a Western blot showing NOXA and MCL-1 levels in cells treated according to the experiment summarized in FIG. 28A at indicated drug concentrations.

FIGS. 29A and 29B show that HMA treatment increases MCL-1 dependency. FIG. 29A provides an overview of an MCL-1 dependency assay. FIG. 29B shows flow cytometry data. The lower panel of FIG. 29B lists % priming under each treatment condition.

FIG. 30 is a bar graph demonstrating that HMA treatment increases sensitivity to MCL-1 suppression.

FIGS. 31A and 31B show results from sequential dosing of alvocidib and HMAs in a MOLM13 model for MDS/sAML. HMA dosing sensitized AML cells to sequential dosing and improved survival in vivo. Alvocidib, azacytidine, and decitabine activity were assessed in the MOLM13 xenograft model. FIG. 31A is a plot of tumor volume over time and FIG. 31B is a plot of percent survival over time. Mice were dosed with alvocidib, qdx2, and/or an HMA, qdx3 on a weekly basis. Doses are indicated in mg/kg (mpk).

FIGS. 32A and 32B show results from aggressive daily dosing of alvocidib and decitabine in the MOLM13 model. Tumor bearing mice were treated. Doses and schedule are indicated. FIG. 32A plots tumor volume following treatment and FIG. 32B plots body weight following treatment. As a single agent, alvocidib achieved tumor growth inhibition (% TGI) of 75.8. Decitabine achieved a % TGI of 58.6 as a single agent. In combination, decitabine and alvocidib achieved a % TGI of 95.8.

FIG. 33 is an illustration of a study schema for an ALV-102 clinical trial including an azacitidine arm. The clinical trial will evaluate azacitidine+/−alvocidib in newly diagnosed intermediate and high-risk myelodysplastic syndromes. Alvocidib will be administered as 1-hour infusion.

FIG. 34 demonstrates that alvocidib as part of an ACM regimen showed clinical activity in AML patients with prior MDS.

FIG. 35 illustrates decitabine-mediated re-expression of NOXA is complementary with MCL-1 repression by alvocidib.

FIG. 36 is an image of a Western blot demonstrating that decitabine effects an increase in NOXA expression.

FIG. 37 demonstrates that administration of decitabine followed by administration of alvocidib results in a synergistic increase in normalized caspase 3/7 activity in an AML cell line. The lower panel of FIG. 37 illustrates a dosing regimen used to assess any synergy between decitabine and alvocidib. Cells were exposed to decitabine (DAC) for 24 hours followed by 24 hours exposure to ALV or Palbo. The upper left panel of FIG. 37 and the upper right panel of FIG. 37 are bar graphs demonstrating a synergy between DAC and AML administered at indicated concentrations according to the protocol shown in the lower panel of FIG. 37.

FIG. 38 shows patient NOXA mRNA expression over the course of treatment.

FIG. 39A is a Western blot obtained from the experiment described in Example 16, and shows that azacitidine (alone) induced NOXA expression and alvocidib (alone) suppressed MCL-1 expression, whereas sequential treatment of azacitidine and alvocidib showed both NOXA induction and MCL-1 suppression compared with a DMSO-treated sample in CD34⁺ MDS bone marrow mononuclear cells (BMMNC) from Patient 0219.

FIG. 39B is a graph of apoptotic versus treatment according to the experiment described in Example 16, and shows that azacitidine (alone) showed 52 and 62% increase in apoptotic activity at the concentration of 0.3 and 0.6 μM, respectively, whereas sequential treatment of azacitidine (0.3 or 0.6 μM) and alvocidib showed 104 and 110% increase of apoptotic activity, respectively, compared with a DMSO-treated sample.

FIG. 40A shows the effect of alvocidib, azacitidine or alvocidib+azacitidine on apoptosis in CD34⁺ MDS BMMNC from a patient sample.

FIG. 40B shows the effect of alvocidib, azacitidine or alvocidib+azacitidine on apoptosis in CD34⁺ BMMNC from another patient sample.

FIG. 41A shows the gating strategy used in the MCL-1 dependency assay described in Example 18.

FIG. 41B is a schematic of the MCL-1 dependency assay described in Example 18.

FIG. 41C is a graph of calibrated percent MCL-1 priming versus patient as a function of azacitidine treatment, and shows a dose-dependent increase in priming observed with 0.3, 1 and 2.5 μM azacitidine treatment across multiple bone marrow samples from patients with MDS.

FIG. 42 is an interim response assessment summary from the clinical trial described in Examples 9-15, and shows the responses and durations of treatment by tumor type.

FIG. 43A shows pharmacokinetic curves for alvocidib from patients in cohort 4 (30 mg/m² bolus+60 mg/m² infusion) of the clinical trial described in Examples 9-15 on C1D8.

FIG. 43B shows pharmacokinetic curves for alvocidib from patients in cohort 5 (75 mg/m² IVI) of the clinical trial described in Examples 9-15 on C1D10.

FIG. 43C is a plot of mean Cₘₐₓ of alvocidib as a function of cohort in the clinical trial described in Examples 9-15.

FIG. 43D is a plot of mean AUC of alvocidib as a function of cohort in the clinical trial described in Examples 9-15.

FIG. 43E shows various treatment trends for cohorts 1-4 during cycle 1 of treatment according to the clinical trial described in Examples 9-15.

FIG. 43F shows various treatment trends for cohorts 1-4 during cycle 2 of treatment according to the clinical trial described in Examples 9-15.

FIG. 43G shows various treatment trends for cohorts 1-4 during cycle 4 of treatment according to the clinical trial described in Examples 9-15.

FIG. 43H shows relative NOXA expression on C1D8 as a function of cohort in the clinical trial described in Examples 9-15.

FIG. 43I shows relative NOXA expression on C1D15 as a function of cohort in the clinical trial described in Examples 9-15.

FIG. 44A is an x-ray diffractogram obtained from XRPD analysis of polymorph Form B.

FIG. 44B shows the differential scanning calorimetry output of heat flow plotted as a function of temperature for polymorph Form B.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, e.g., arresting, reducing or delaying the development of the disease or a relapse thereof or at least one clinical or sub-clinical symptom thereof, or (3) relieving the disease, e.g., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

Pharmaceutically acceptable salts can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

The term “solvate” means a physical association of a compound with one or more solvent molecules, whether organic or inorganic. This physical association includes hydrogen bonding. In certain instances, the solvate will be capable of isolation, for example, when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution phase and isolable solvates. Examples of solvates include, but are not limited to, hydrates, ethanolates, methanolates, and isopropanolates. Methods of solvation are generally known in the art. Compounds referred to herein (e.g., alvocidib, or a prodrug thereof, or a pharmaceutically acceptable salt of the foregoing; azacitidine, or a prodrug thereof, or a pharmaceutically acceptable salt of the foregoing; decitabine, or a prodrug thereof, or a pharmaceutically acceptable salt of the foregoing) can, in certain embodiments, be present in solvated form, as a solvate (e.g., hydrate).

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Many therapies are administered on a treatment cycle, or cycle. As used herein, “treatment cycle” and “cycle” are used interchangeably to refer to a therapy (e.g., schedule or course of therapy comprising periods of treatment) that is repeated on a regular or substantially regular schedule. The length of a treatment cycle is determined by the treatment being administered, but can be 1, 2, 3, 4, 5, 6, 7, 8, 9, 10, 11, 12, 13, 14, 15, 16, 17, 18, 19, 20, 21, 22, 23, 24, 25, 26, 27 or 28 days, or 4, 5, 6, 7, 8, 9, 10, 11 or 12 weeks. In some embodiments, a treatment cycle is 21 days. In some embodiments, a treatment cycle is 28 days.

Many treatment cycles comprise periods of treatment and periods of no treatment. As used herein, a “drug holiday” refers to a period of time during which the subject is not given the agent or agent(s) that make up the therapy (e.g., alvocidib, or a prodrug thereof, or a pharmaceutically acceptable salt of the foregoing, and/or a hypomethylating agent). In some embodiments, the subject may not be given any therapeutic agent during a drug holiday. In other embodiments, the subject may be administered prophylactic agents or palliative care during a drug holiday.

Therapies and/or particular therapeutic agents (e.g., alvocidib, or a prodrug thereof, or a pharmaceutically acceptable salt of the foregoing, in particular, a compound of structural formula I), can also be administered continuously.

“MCL-1-dependent” refers to the subset of cancers wherein myeloid cell leukemia 1 (MCL-1) is the primary driver of suppressing apoptosis. Typically, MCL-1 dependency promotes blast survival, and is associated with treatment resistance and relapse. MCL-1 dependence can be assessed, for example, by contacting a subject's cancer cell with a profiling peptide, as described in International Publication Nos. WO 2016/172214 and WO 2018/119000, the relevant contents of which are incorporated herein by reference in their entireties. Examples 5 and 18 herein describe the assessment of MCL-1 dependence in various populations of hematologic cancer cells, including blasts, from MDS patient samples.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

A “DNA methyltransferase inhibitor” is an agent having dual activity as an inhibitor of DNA methyltransferase (i.e., a hypomethylating agent (“HMA”)) and activity as a DNA-damaging agent. Exemplary DNA methyltransferase inhibitors are incorporated into DNA (e.g., DNA in a cancer cell), thereby inhibiting DNA methyltransferase and leading to DNA damage and apoptosis. Exemplary DNA methyltransferase inhibitors include nucleoside analogues, such as azanucleosides.

“Azanucleosides” are analogues of natural occurring nucleosides, wherein at least one carbon atom has been replaced with a nitrogen atom. Non-limiting examples of azanucleosides include azacitidine (e.g., ONUREG®), or a prodrug thereof (such as a phosphate prodrug or 2′,3′,5′-triacetyl-5-azacitidine), and decitabine (e.g., INQOVI®), or a prodrug thereof. Phosphate prodrugs of azacitidine suitable for use in the present methods are disclosed in International Publication No. WO 2011/153374, which is hereby incorporated by reference in its entirety. For example, one phosphate prodrug of azacitidine has the formula:

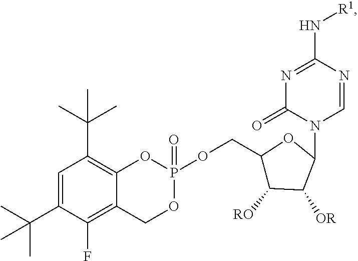

or a pharmaceutically acceptable salt or solvate thereof, wherein R and R¹ are independently H or CO₂(C₁-C₆ alkyl) (e.g., each R is H and R¹ is CO₂(C₅-alkyl)). Prodrugs of azacitidine, including phosphate prodrugs and 2′,3′,5′-triacetyl-5-azacitidine, can be administered orally. Exemplary azanucleosides include hypomethylating agents (HMAs) azacitidine and decitabine, which have the following structures, respectively:

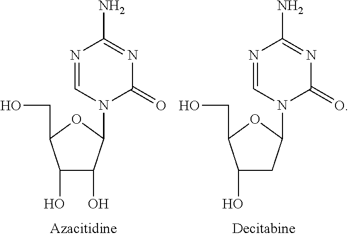

A “cyclin-dependent kinase inhibitor” is an agent which inhibits the activity of cyclin dependent kinases (CDKs), including CDK1, CDK2, CDK3, CDK4, CDK5, CDK6, CDK7, CDK8, CDK9 and CDK11. Exemplary CDK inhibitors inhibit the expression of MCL-1. Exemplary CDK inhibitors include, but are not limited to, alvocidib, dinaciclib, olomoucine, roscovitine, purvalanol, paullones, palbociclib, thio/oxoflavopiridols, oxindoles, aminothiazoles, benzocarbazoles, pyrimidines and seliciclib.

“Alvocidib” (also known as “flavopiridol”) is a synthetic flavone having the following structure:

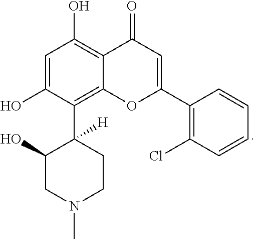 [SMILES: CN1CC[C@H](c2c(O)cc(O)c3c(=O)cc(-c4ccccc4Cl)oc23)[C@H](O)C1]

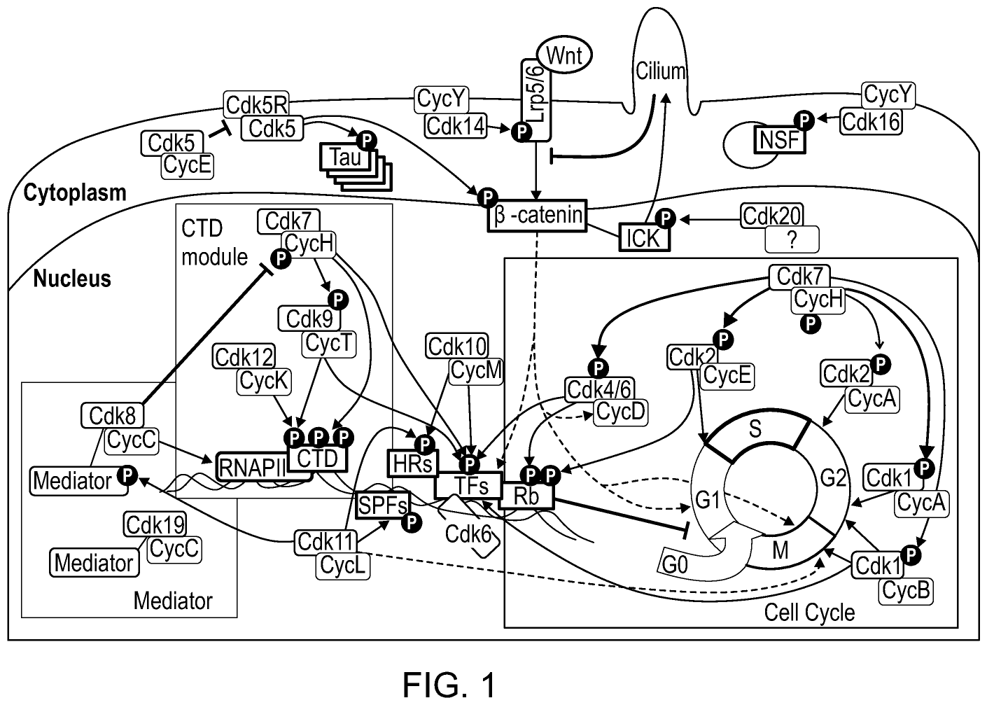

A schematic illustration providing an overview of CDK functions in a cell is shown in FIG. 1. Malumbres M. Genome Biol. 2014; 15(6):122. Each CDK (in orange boxes) is shown in a complex with its major partner (green)—for clarity, only a few substrates are depicted. Most CDKs function in the nucleus (orange background), whereas a few family members are attached to the cell membrane or display cytoplasmic activities (blue background). Classical cell cycle CDKs—Cdk4, Cdk6, Cdk2 and Cdk1—regulate the transitions through the different phases of the cell-division cycle. These activities are at least partially mediated by the control of multiple transcription factors (TFs) or regulatory elements such as the retinoblastoma protein (Rb). Cdk10 and Cdk11 also control transcription by phosphorylating TFs, hormone receptors and associated regulators (HRs), or splicing factors (SPFs). Cdk7, Cdk9 and Cdk12 directly phosphorylate the C-terminal domain (CTD) of RNA polymerase II (RNAPII), thus modulating the different phases of generation of transcripts. The Mediator complex is specifically regulated by Cdk8 or the highly related Cdk19. Cdk7 functions as a CDK-activating kinase (CAK) by directly phosphorylating several of the CDKs mentioned above. Cdk5 displays many functions in the cell, but it is better known for its function in the control of neuron-specific proteins such as Tau. The members of the Cdk14 subfamily, such as Cdk14 itself or Cdk16, are activated at the membrane by cyclin Y and also participate in many different pathways, such as Wnt-dependent signaling or signal transduction in the primary cilium. For clarity, many interactions between CDKs and other partners, substrates or cellular processes are not shown—for instance, Cdk1 can bind to other cyclins and can also phosphorylate more than 100 substrates during mitotic entry that are not indicated here. CAK, CDK-activating kinase; CDK, cyclin-dependent kinase; CTD, C-terminal domain; Rb, retinoblastoma protein; RNAPII, RNA polymerase II; SPF, splicing factor; TF, transcription factor.

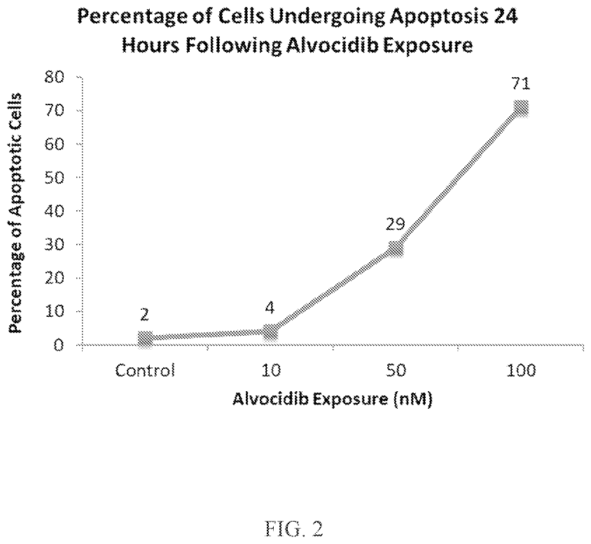

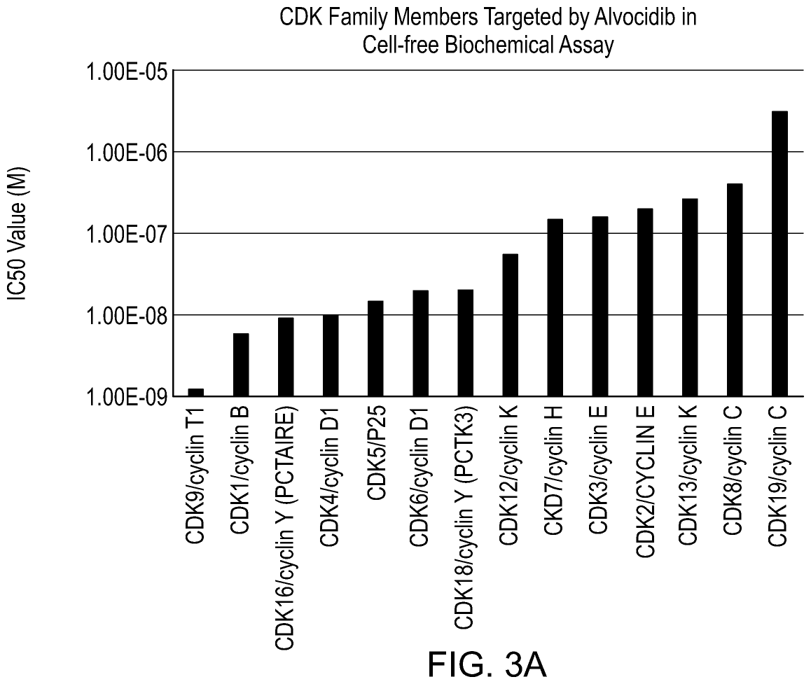

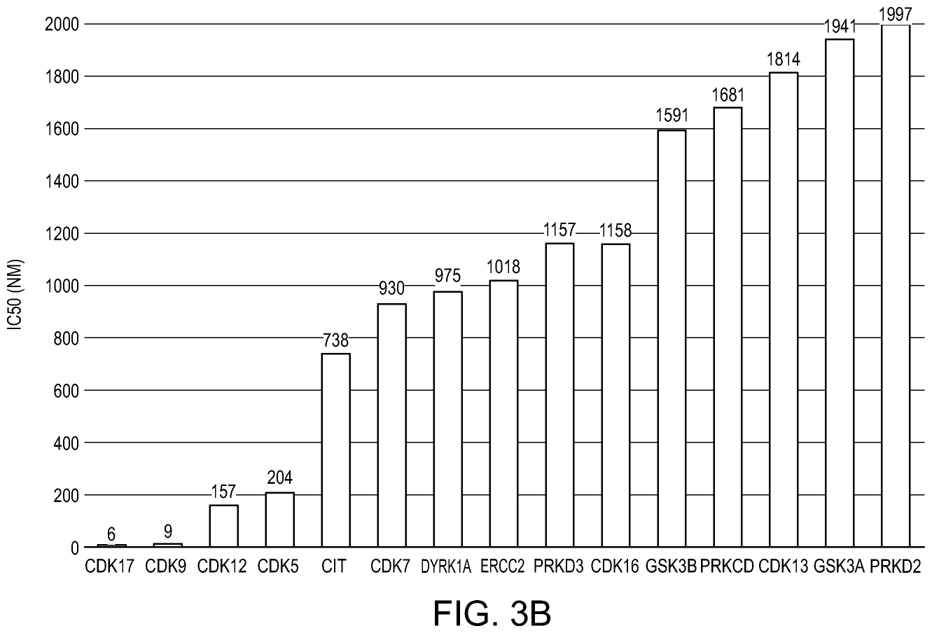

Alvocidib causes rapid cell death independent of the cell cycle. As shown in an in vitro study, alvocidib-induced cell apoptosis is rapid and not linked to cell cycle arrest. Mayer F. Invest New Drugs. 2005; 23:205-211. FIG. 2 is a plot of the percentage of cells undergoing apoptosis (y-axis) following 24 hours alvocidib exposure in nM (x-axis). In the clinic, three-day dosing of alvocidib resulted in an average reduction of circulating blasts of greater than 75%. FIGS. 3A and 3B are bar graphs showing IC50 values for alvocidib. FIG. 3A is a bar graph of IC50 values (M) for different CDK family members targeted by alvocidib in a cell-free biochemical assay. FIG. 3B is a bar graph of IC50 values (nM) for kinases inhibited by alvocidib at cellular concentrations below 2 μM. Methodology: Kinobeads (proteomics); Cell Lysate mixture: K-562, COLO 205, MV-4-11, SK-N-BE; Klaeger S, et al. Science. 2017; 358(6367).pii: eaan4368 In vivo studies further demonstrate that three-day dosing of alvocidib results in an average reduction of circulating blasts of greater than 75%. Blum W. Haematologica. 2010; 1098-1105

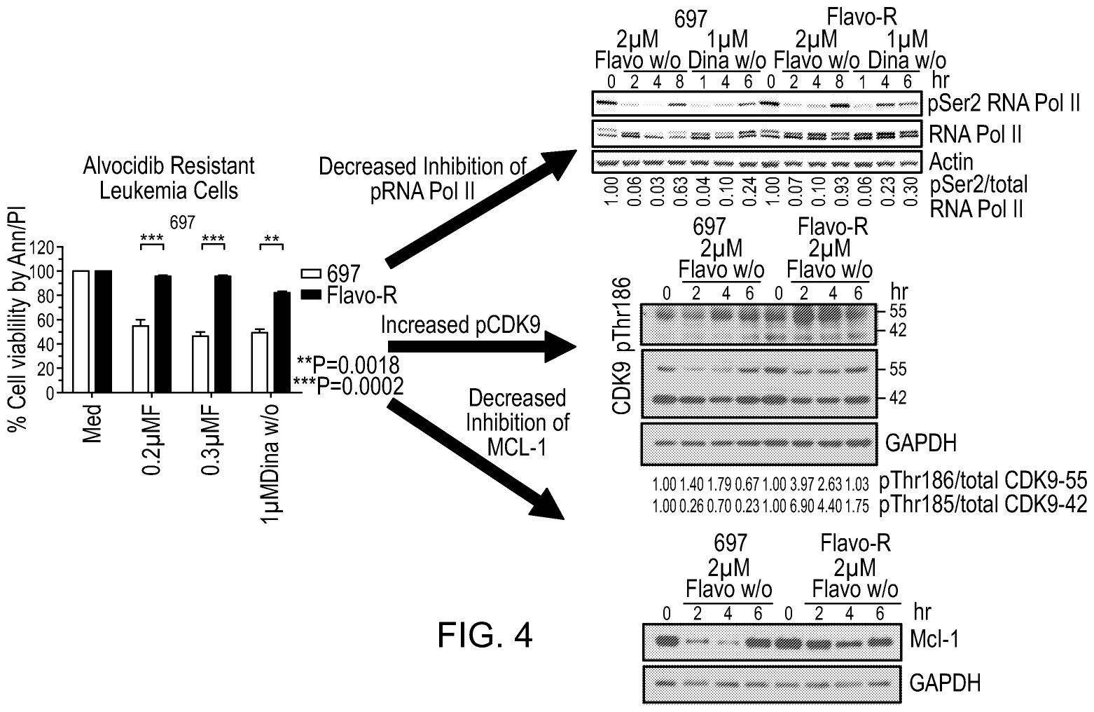

As demonstrated in FIG. 4, up-regulation of CDK9 kinase activity and MCL-1 stability contribute to acquired resistance to cyclin-dependent kinase inhibitors in leukemia. Yey Y Y, et al. Oncotarget. 2014:6(5):2667-2679 The bar graph in the left panel of FIG. 4 shows that leukemia cells acquired resistance to flavopiridol-induced cell death in vitro. 697 parental and flavopiridol-resistant (Flavo-R) cells were treated with continuous flavopiridol (0.2 M or 0.3 μM), or 1 μM dinaciclib with 2-hour exposure and washout (w/o). Cell viability was measured post 24 hours by annexin V-FITC and PI-PE stains, followed by flow cytometry. Flavo-R also developed cross-resistance to dinaciclib with a significant survival advantage over parental cells at 24 hours post to dinaciclib treatment. Due to the similar effects of continuous 0.2 M or 0.3 M flavopiridol treatment, p-values represent the average effect for both doses. The top right panel of FIG. 4 shows that phosphorylation of Ser2 of RNA Pol II CTD is more resistant against flavopiridol's drug action. 697 parental and Flavo-R cells were treated with either 2 μM flavopiridol with 4-hour exposure and washout (w/o), or 1 μM dinaciclib with 2-hour exposure and washout (w/o) and collected at various time points as indicated in the top right panel. Protein lysates were prepared and subjected to immunoblotting for phosphorylation of Ser2 of RNA Pol II, total RNA Pol II and actin. Consistently, Flavo-R revealed more robust Ser2 phosphorylation with flavopiridol and dinaciclib, implicating higher activity of RNA Pol II. The upper right panel also suggests that Flavo-R mechanistically established resistance to dinaciclib in vitro in concert with observations in cell cytotoxicity described in reference to the left panel. Densitometry was applied to quantify intensity of immunoreactive bands. Arbitrary numbers are shown at the bottom of the upper right panel. The middle panel on the right of FIG. 4 shows that CDK9 kinase activity is upregulated to promote RNA Pol II activity, counter to the drug mechanism of flavopiridol. 697 parental and resistant cells were treated with 4 hr-exposure of 2 μM flavopiridol and harvested for protein lysate at pre (0 hr), 2, 4 and 6 hr. Lysates were subjected to immunoblotting for phospho-Thr186 in the CDK9 activation loop as a surrogate for CDK9 kinase activity. Densitometry was applied to quantify the intensity of immunoreactive bands for phospho-Thr186 of both CDK9 isoforms, which were normalized to total CDK9 and the arbitrary numbers are shown at the bottom of the figure. CDK9 kinase activity of both isoforms was further increased with flavopiridol in Flavo-R. The lower right panel of FIG. 4 shows that MCL-1 protein levels are more stable to antagonize the flavopiridol-mediated depletion in Flavo-R. Immunoblotting was applied to detect MCL-1 protein levels in protein lysates collected from cells treated with 2 M flavopiridol for 4 hours and washout (w/o).

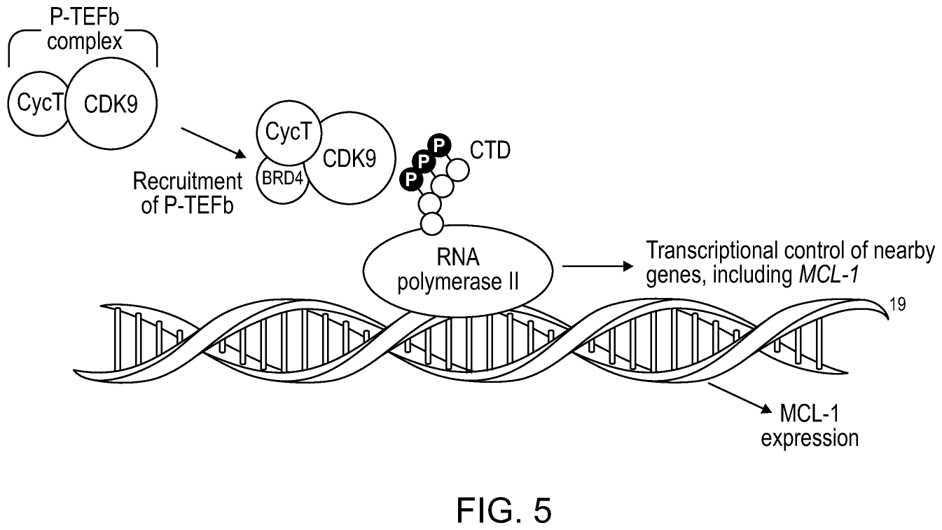

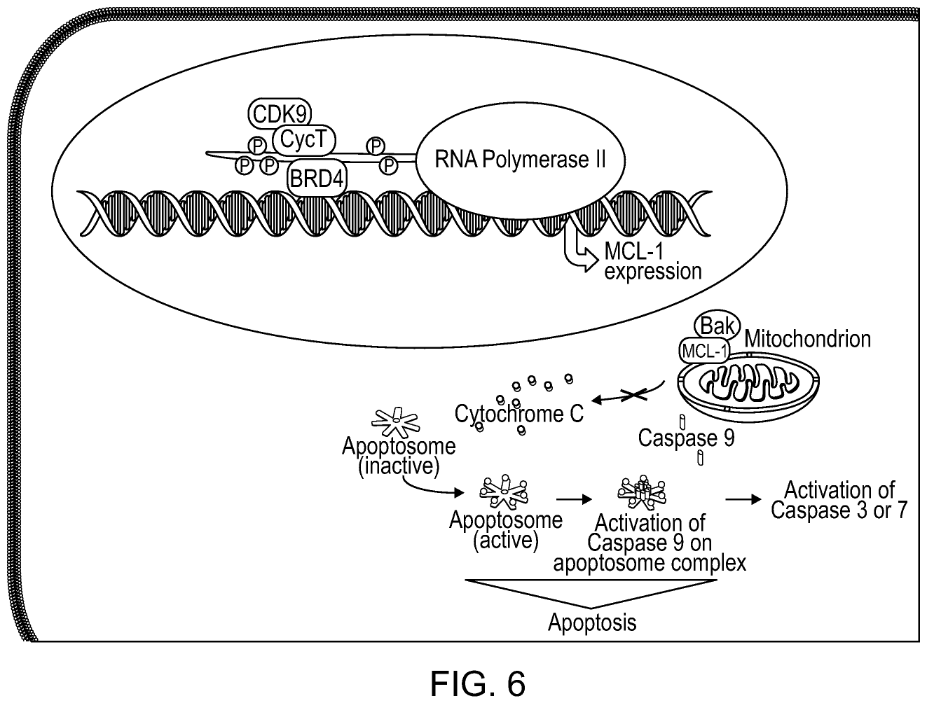

As a potent inhibitor of CDK9, alvocidib disrupts super enhancer activity via CDK9 inhibition. As shown in a schematic in FIG. 5, Alvcodib downregulates CDK9-driven transcription of super enhancer-regulated genes, such as c-Myc and MCL-1. FIG. 6 is a diagram showing how CDK9 is part of a super-enhancer complex regulating RNA polymerase II and drives the expression of MCL-1, a member of the BCL-2 family that inhibits apoptosis.

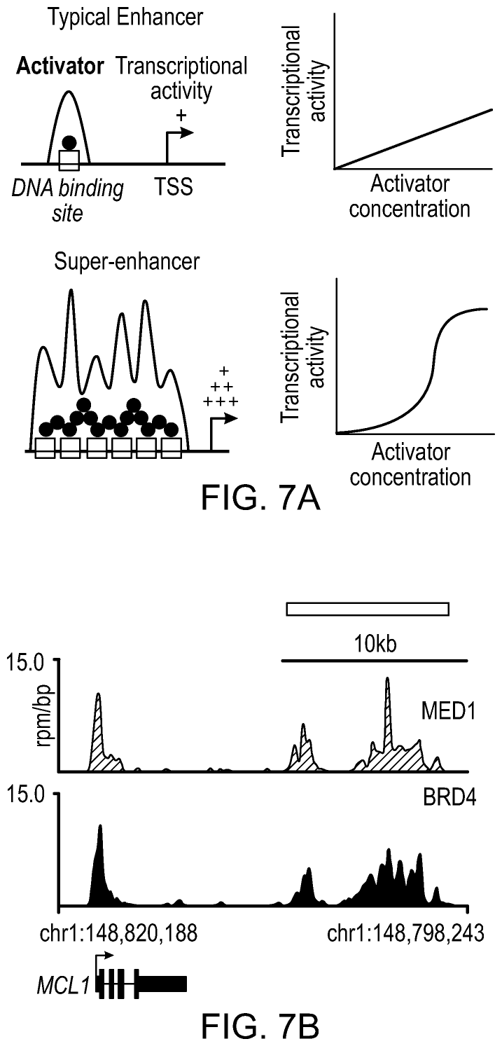

FIG. 4 together demonstrate that MCL-1 is regulated by a super-enhancer in AML cells. Cooperative interactions of enhancer-associated factors at super-enhancers leads to both higher transcriptional output and increased sensitivity to factor concentration, as shown in FIG. 7A. The left panel of FIG. 7A shows (left) gene tracks of a typical enhancer (black) and of a super-enhancer (red) of ChIP-seq occupancy upstream of a transcription start site (TSS) for a representative gene controlled by a typical enhancer (top) and for a gene controlled by a super-enhancer (bottom). Instances of the symbol “+” represents relative transcriptional activity. The right panel of FIG. 7A illustrates how transcriptional activity responds to changes in activator concentration for a typical enhancer and for a super-enhancer. FIG. 7B shows gene tracks of MED1 (top) and BRD4 (bottom) ChIP-seq occupancy overlapping with and downstream of the MCL1 gene. A super-enhancer-containing region downstream of MCL1 is depicted with a gray box. The y-axis shows signal of ChIP-seq occupancy in units of reads per million mapped reads per bp (rpm/bp).

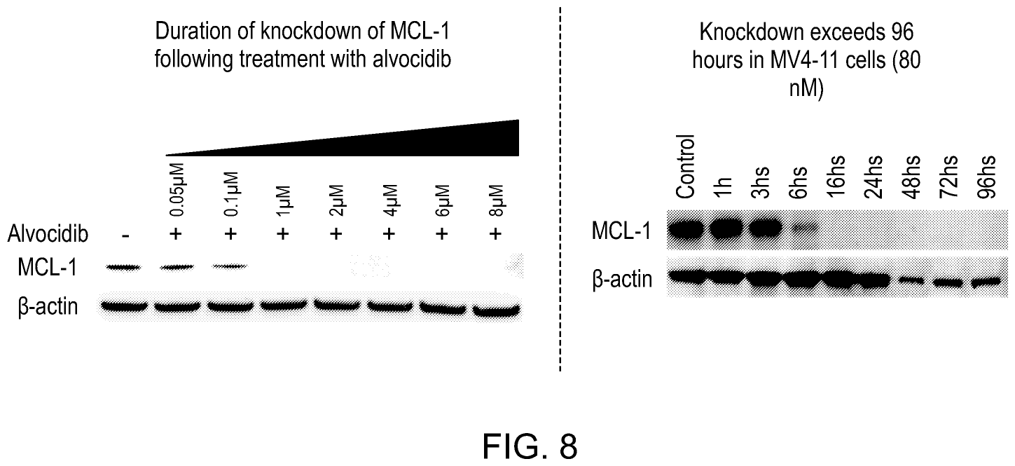

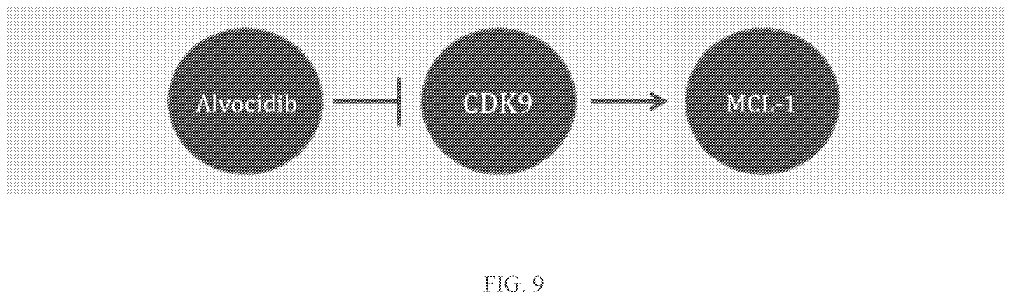

Alvocidib inhibits protein expression of MCL-1, as shown in FIG. 8. The left panel of FIG. 8 is a Western blot demonstrating a dose-dependent decrease in MCL-1 in response to alvocidib. The right panel of FIG. 8 is a Western blot showing a progressive decrease over time of MCL-1 in MV4-11 cells treated with 80 nM alvocidib. The right panel of FIG. 8 demonstrates that knock-down of MCL-1 exceeds 96 hours. FIG. 9 is an illustration of a proposed mechanism by which alvocidib inhibits expression of MCL-1 by inhibiting CDK9.

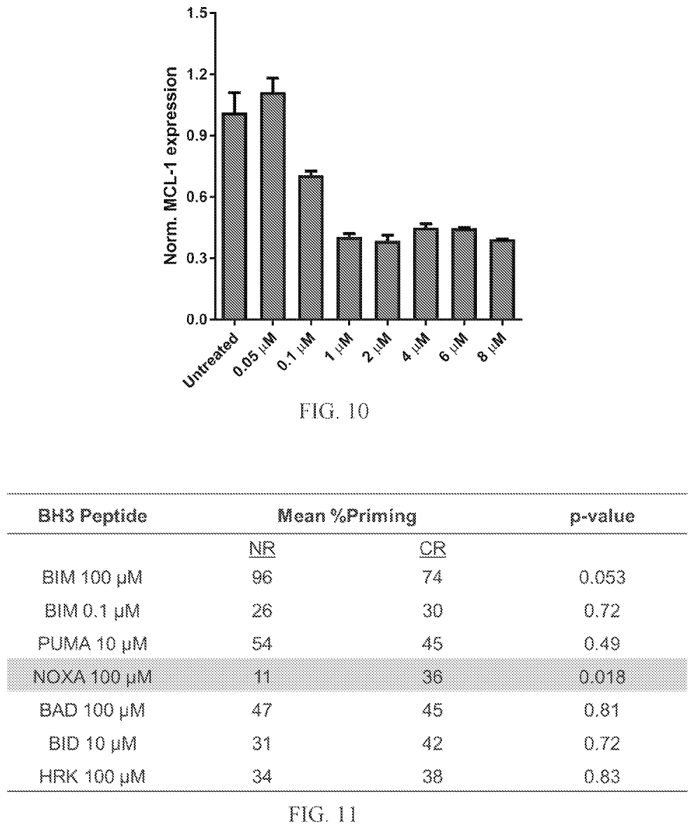

Alvocidib suppresses MCL-1 protein and mRNA expression. FIG. 10 is a bar graph of normalized MCL-1 gene expression (measured as mRNA) at indicated concentrations of alvocidib showing MCL-1 suppression by alvocidib. Furthermore, NOXA priming (MCL-1 dependence) is associated with alvocidib response, as shown in FIG. 11, which shows the results of a BH3 profile (mitochondrial profiling) of cells treated with indicated concentrations of alvocidib. Significance values (p-value) are provided.

“Prodrug” is meant to indicate a compound that may be converted under physiological conditions or by solvolysis to a biologically active compound. Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammal (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein for their teachings regarding the same. The term “prodrug” is also meant to include any covalently bonded carriers, which release the active compound in vivo when such prodrug is administered to a subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, phosphate, acetate, formate and benzoate derivatives of a hydroxy functional group, or acetamide, formamide and benzamide derivatives of an amine functional group in the active compound, and the like.

Examples of prodrugs of alvocidib are described in International Publication Nos. WO 2016/187316 and WO 2018/094275, which are incorporated herein by reference in their entireties for their teachings regarding the same. In some embodiments, the prodrug of alvocidib is a phosphate prodrug of alvocidib. In some instances, the prodrug of alvocidib can be a compound of the following structure:

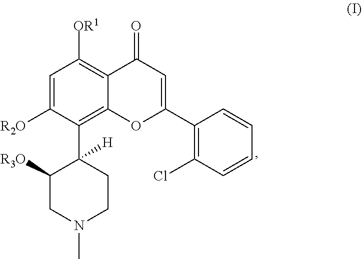

or a pharmaceutically acceptable salt thereof, wherein one of R¹, R² and R³ is —P(═O)(OH)₂, and the other two of R¹, R² and R³ are each —H. In some instances, the prodrug of alvocidib can be the compound of the following structure:

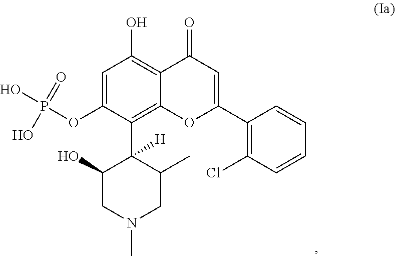 [SMILES: CC1CN(C)C[C@@H](O)[C@H]1c1c(OP(=O)(O)O)cc(O)c2c(=O)cc(-c3ccccc3Cl)oc12]

or a pharmaceutically acceptable salt thereof. The compounds of Structural Formulas I and Ia are orally bioavailable. Thus, the compounds of Structural Formulas I and Ia, or a pharmaceutically acceptable salt of the foregoing, can be administered orally, and compositions comprising a compound of Structural Formula I or Ia, or a pharmaceutically acceptable salt thereof, can be formulated for oral administration.

It will be appreciated that a prodrug of alvocidib, such as the compound of structural formula Ia, can exist in zwitterionic form, such as the zwitterionic form represented by the following structure:

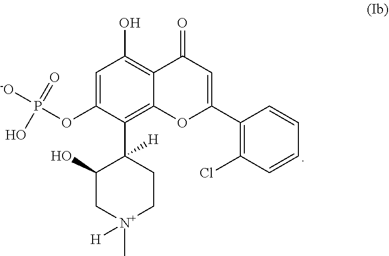

In any of the embodiments of a prodrug herein, the prodrug (e.g., compound of structural formula Ia) can be present in its free form or zwitterionic form, or a pharmaceutically acceptable salt form. Thus, in some embodiments, the prodrug is a compound of structural formula Ia, or a zwitterionic form or pharmaceutically acceptable salt thereof, e.g., a compound of structural formula Ib.

Crystalline forms of a compound of structural formula Ia, or a zwitterionic form or pharmaceutically acceptable salt thereof, e.g., a compound of structural formula Ib, are disclosed in International Publication No. WO 2020/117988, the entire contents of which are incorporated herein by reference.

“Crystalline,” as used herein, refers to a homogeneous solid formed by a repeating, three-dimensional pattern of atoms, ions or molecules having fixed distances between constituent parts. The unit cell is the simplest repeating unit in this pattern. Notwithstanding the homogenous nature of an ideal crystal, a perfect crystal rarely, if ever, exists. “Crystalline,” as used herein, encompasses crystalline forms that include crystalline defects, for example, crystalline defects commonly formed by manipulating (e.g., preparing, purifying) the crystalline forms described herein. A person skilled in the art is capable of determining whether a sample of a compound is crystalline notwithstanding the presence of such defects.

“Polymorph,” as used herein, refers to a crystalline form of a compound characterized by a distinct arrangement of its molecules in a crystal lattice. Polymorphs can be characterized by analytical methods such as x-ray powder diffraction (XRPD), differential scanning calorimetry (DSC) and thermogravimetric analysis.

The crystalline forms and/or polymorphs described herein can be substantially pure. As used herein, “substantially pure,” used without further qualification, means the indicated compound has a purity greater than 90 weight percent, for example, greater than 90, 91, 92, 93, 94, 95, 96, 97, 98, or 99 weight percent, and also including a purity equal to about 100 weight percent, based on the weight of the compound. The remaining material comprises other form(s) of the compound, and/or reaction impurities and/or processing impurities arising from its preparation (e.g., alvocidib). Purity can be assessed using techniques known in the art, for example, using an HPLC assay described herein. “Substantially pure” can also be qualified as in “substantially pure of other physical forms of a compound of structural formula I, or a tautomer or zwitterionic form thereof” or “substantially pure of alvocidib.” When qualified thus, “substantially pure” means that the indicated compound contains less than 10%, preferably less than 5%, more preferably less than 3%, most preferably, less than 1% by weight of the indicated impurity (e.g., any other physical forms of an indicated crystalline form of a compound; alvocidib).

An XRPD pattern or DSC thermogram that is “substantially in accordance” with one or more figures herein showing an XRPD pattern or diffractogram or DSC thermogram, respectively, is one that would be considered by one skilled in the art to represent the same single crystalline form of the compound of structural formula I, or a tautomer or zwitterionic form or pharmaceutically acceptable salt thereof, as the sample of the compound that provided the pattern or diffractogram or thermogram of one or more figures provided herein. Thus, an XRPD pattern or DSC thermogram that is substantially in accordance may be identical to that of one of the figures or, more likely, may be somewhat different from one or more of the figures. For example, an XRPD pattern that is somewhat different from one or more of the figures may not necessarily show each of the lines of the diffraction pattern presented herein and/or may show a slight change in appearance or intensity of the lines or a shift in the position of the lines. These differences typically result from differences in the conditions involved in obtaining the data or differences in the purity of the sample used to obtain the data. A person skilled in the art is capable of determining if a sample of a crystalline compound is of the same form as or a different form from a form disclosed herein by comparison of the XRPD pattern or DSC thermogram of the sample and the corresponding XRPD pattern or DSC thermogram disclosed herein.

The crystalline forms provided herein can also be identified on the basis of differential scanning calorimetry (DSC) and/or thermogravimetric analysis (TGA). DSC is a thermoanalytical technique in which the difference in the amount of heat required to increase the temperature of a sample is measured as a function of temperature. DSC can be used to detect physical transformations, such as phase transitions, of a sample. For example, DSC can be used to detect the temperature(s) at which a sample undergoes crystallization, melting or glass transition. It is to be understood that any temperature associated with DSC specified herein, with the exception of the DSC temperatures in the Figures or Examples, means the specified value±5° C. or less. For example, when an embodiment or a claim specifies an endothermic peak at 264° C., this is to be understood to mean 264° C.±5° C. or less, that is a temperature of from 259° C. to 269° C. In preferred embodiments, a DSC is the specified value±3° C. or less, in more preferred embodiments, ±2° C. or less.

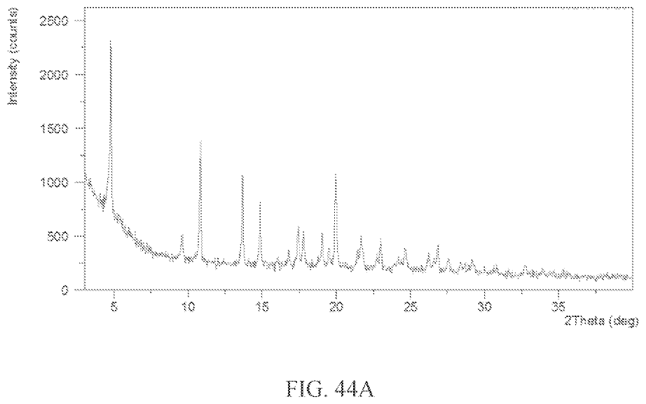

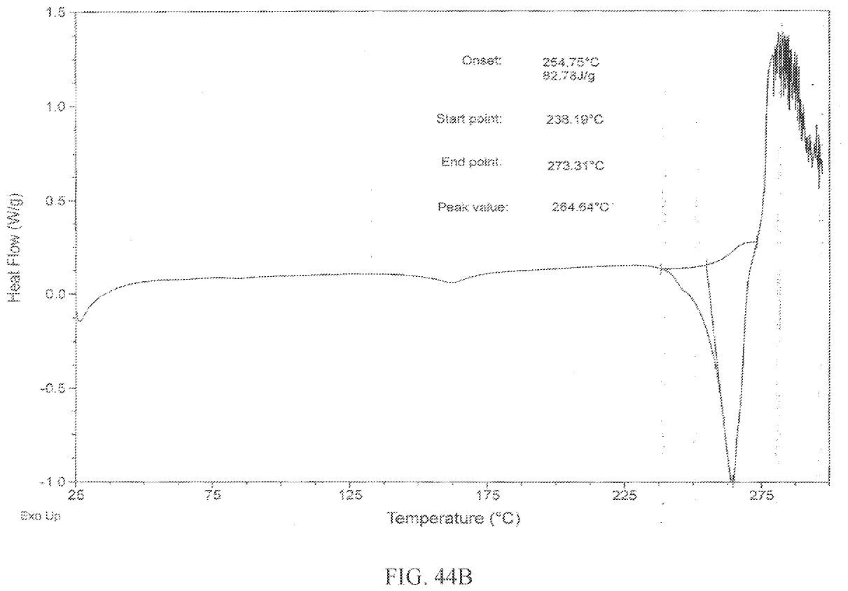

In some embodiments, a compound of structural formula Ia, or a zwitterionic form or pharmaceutically acceptable salt thereof, e.g., a compound of structural formula Ib, comprises, consists essentially of or consists of Form B. In some embodiments, the crystalline form (e.g., Form B) is substantially pure (e.g., of other physical forms of the compound of structural formula I, or a tautomer or zwitterionic form or pharmaceutically acceptable salt thereof, of impurities; of alvocidib). Form B has the structure of structural formula Ib and is characterized, in some embodiments, by an x-ray powder diffraction (XRPD) pattern comprising at least three peaks (e.g., three peaks, at least four peaks, four peaks, at least five peaks, five peaks, six peaks) at 2-theta angles selected from the group consisting of 4.8±0.2°, 10.8±0.2°, 13.7±0.2°, 14.9±0.2°, 20.0±0.2° and 24.6±0.2°. In some embodiments, Form B is characterized by an XRPD pattern comprising peaks at the following 2-theta angles: 10.8±0.2°, 14.9±0.2° and 20.0±0.2°. In some embodiments, Form B is characterized by an XRPD pattern comprising peaks at the following 2-theta angles: 4.8±0.2°, 10.8±0.2°, 14.9±0.2° and 20.0±0.2°. In some embodiments, Form B is characterized by an XRPD pattern comprising peaks at the following 2-theta angles: 4.8±0.2°, 10.8±0.2°, 13.7±0.2°, 14.9±0.2° and 20.0±0.2°. In some embodiments, Form B has an XRPD pattern substantially in accordance with that depicted in FIG. 44A. In some embodiments, Form B is characterized by a DSC thermogram comprising an endothermic peak at about 264° C. In some embodiments, Form B is characterized by a DSC thermogram substantially in accordance with that depicted in FIG. 44B.

In one embodiment, a polymorph of a compound of structural formula Ia, or a zwitterionic form or pharmaceutically acceptable salt thereof, e.g., a compound of structural formula Ib, has an X-ray powder diffraction pattern comprising the following:
D space (Å):
18.3±0.09
8.1±0.06
6.4±0.08
5.9±0.06
4.4±0.05
   expressed in terms of “D” spacing. In a related embodiment, the polymorph has an X-ray powder diffraction pattern comprising the following:
D space (Å):
18.38±0.003
8.15±0.008
6.47±0.002
5.95±0.007
4.44±0.006
   expressed in terms of “D” spacing. In yet another related embodiment, the polymorph has an X-ray powder diffraction pattern comprising the following:
D space (Å):
18.382
8.157
6.472
5.956
4.445
   expressed in terms of “D” spacing.

In one embodiment, a polymorph of a compound of structural formula Ia, or a zwitterionic form or pharmaceutically acceptable salt thereof, e.g., a compound of structural formula Ib, is a crystalline form having a monoclinic space group P2₁ with lattice parameters of: a=6.46(1) Å, b=9.07(2) Å, c=18.25(4) Å, and β=95.457(2)°; and a volume of 1066.11(4) Å³. In another embodiment, the polymorph of a compound of structural formula Ia, or a zwitterionic form or pharmaceutically acceptable salt thereof, e.g., a compound of structural formula Ib, is a crystalline form having a monoclinic space group P2₁ with lattice parameters of: a=6.4695(1) Å, b=9.0692(2) Å, c=18.2530(4) Å, and β=95.457(2)°; and a volume of 1066.11(4) Å³.

Without wishing to be bound by theory, it is thought that the water content of the polymorph can have a significant effect on the purity and storage stability of the polymorph. That is, the polymorph can undergo a hydrolysis reaction that converts that phosphate moiety to a hydroxyl group. As such, an impurity may be present in the form of hydrolyzed structural formula Ia (i.e., alvocidib). However, it was unexpectedly discovered that keeping the water content of the polymorph and any subsequently formed compositions low provided an active substance (e.g., Form B) with much more robust stability.

Accordingly, in some embodiments, the polymorph of a compound of structural formula Ia, or a zwitterionic form or pharmaceutically acceptable salt thereof, e.g., a compound of structural formula Ib, has an initial purity of at least 99.5% and a subsequent purity of at least 99.5% after being stored from about 12 hours up to about 7 days above a temperature at about 25° C.±2° C. at a relative humidity of 60%. In some embodiments, the subsequent purity is at least 99.5% after being stored for greater than about 7 days at about 25° C.±2° C. at a relative humidity of 60%. In other embodiments, the subsequent purity is at least 99.5% after being stored for greater than about 30 days at about 25° C.±2° C. at a relative humidity of 60%. In some of the foregoing embodiments, the initial purity and subsequent purity are as determined by HPLC.

In some of the foregoing embodiments, the polymorph has water content less than 0.50%, as determined by Karl Fischer titration. For example, in some embodiments, the polymorph has water content less than 0.45%, less than 0.40%, less than 0.35%, less than 0.30%, less than 0.25%, less than 0.20%, less than 0.15%, or less than 0.10%, as determined by Karl Fischer titration.

Hypomethylating agents (HMAs) azacitidine and decitabine are approved therapies for patients with myelodysplastic syndrome (MDS). Both HMAs exert biological activity via DNA damage and inhibition of DNA methyltransferases (DNMTs). DNMT inhibition is hypothesized to induce re-expression of key pro-apoptotic proteins such as NOXA. NOXA sequesters the anti-apoptotic protein MCL-1, preventing association with mitochondrial pore-forming proteins BAX/BAK. As described above, alvocidib is a potent cyclin dependent kinase 9 (CDK9) inhibitor and can block CDK9-dependent MCL-1 expression regulated by RNA polymerase II (RNA Pol II).

Decitabine, for example, is indicated for MDS, including previously treated and untreated, de novo and secondary MDS of all French-American-British (FAB) subtypes (refractory anemia, refractory anemia with ringed sideroblasts, refractory anemia with excess blasts, refractory anemia with excess blasts in transformation, and chronic myelomonocytic leukemia) and intermediate-1, intermediate-2, and high-risk International Prognostic Scoring System. The recommended dose of decitabine is 15 mg/m² by continuous intravenous infusion over three hours, repeated every eight hours for three days, on a six-week cycle. Decitabine can also be administered at a dose of 20 mg/m² by continuous intravenous infusion over one hour, repeated daily for five days, on a four-week cycle.

INQOVI® (also referred to as ASTX727) is a combination of decitabine and cedazuridine, indicated for treatment of adult patients with myelodysplastic syndromes (MD), including previously treated and untreated, de novo and secondary MDS with the following French-American-British subtypes (refractory anemia, refractory anemia with ringed sideroblasts, refractory anemia with excess blasts, and chronic myelomonocytic leukemia [CMML]) and intermediate-1, intermediate-2, and high-risk International Prognostic Scoring System group. The recommended dose of INQOVI® is one tablet (containing 35 mg decitabine and 100 mg cedazuridine), administered orally once daily on Days 1 through 5 of each 28-day cycle.

Azacitidine is indicated for patients with the following FAB myelodysplastic syndrome (MDS) subtypes: Refractory anemia (RA) or refractory anemia with ringed sideroblasts (RARS) (if accompanied by neutropenia or thrombocytopenia or requiring transfusions), refractory anemia with excess blasts (RAEB), refractory anemia with excess blasts in transformation (RAEB-T), and chronic myelomonocytic leukemia (CMMoL). The recommended starting dose of azacitidine for the first treatment cycle, for all patients regardless of baseline hematology values, is 75 mg/m² daily for 7 days, to be administered by subcutaneous injection or intravenous infusion, on a four-week cycle for a minimum of 4 to 6 cycles. After 2 cycles, the dose of azacitidine may be increased to 100 mg/m².

An oral formulation of azacitidine, ONUREG® (also referred to as CC-486), is indicated for continued treatment of adult patients with AML who achieved first complete remission or complete remission with incomplete blood count recovery following intensive induction chemotherapy and are not able to complete intensive curative therapy. ONUREG® is supplied as film-coated tablets containing 200 mg or 300 mg azacitidine for oral use. The recommended dose of ONUREG® is 300 mg, administered orally once daily on Days 1 through 14 of each 28-day cycle.

Alvocidib is under active clinical investigation and has demonstrated high complete response rates in newly diagnosed and relapsed refractory AML patients when administered in combination with cytarabine and mitoxantrone (Zella 201 trial). Given the dual NOXA/MCL-1-targeting ability of combining alvocidib and HMAs, the combination may synergize therapeutically in the treatment of non-clinical models of AML and MDS by means of transcriptional induction of NOXA and repression of MCL-1 expression. Preclinical experiments with alvocidib+HMA demonstrate reduced RNA pol II phosphorylation, NOXA gene methylation, NOXA and MCL-1 mRNA and protein expression, and increased apoptosis, cell viability, tumor growth inhibition and survival in AML cell lines and MDS patient derived bone marrow cells (BMMC). In addition, experiments are planned to evaluate genetically engineered mouse models, MDS cell lines and primary MDS cells in vitro and in vivo to determine the therapeutic response with alvocidib+HMA compared to either agent alone. Readouts will include MDS burden, survival, bone marrow and peripheral blood recovery in the various models.

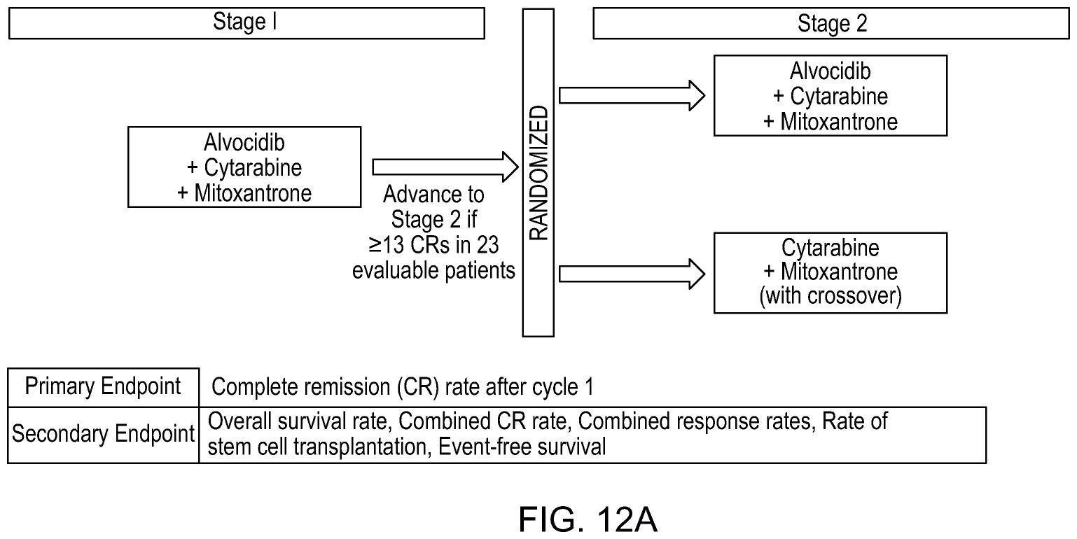

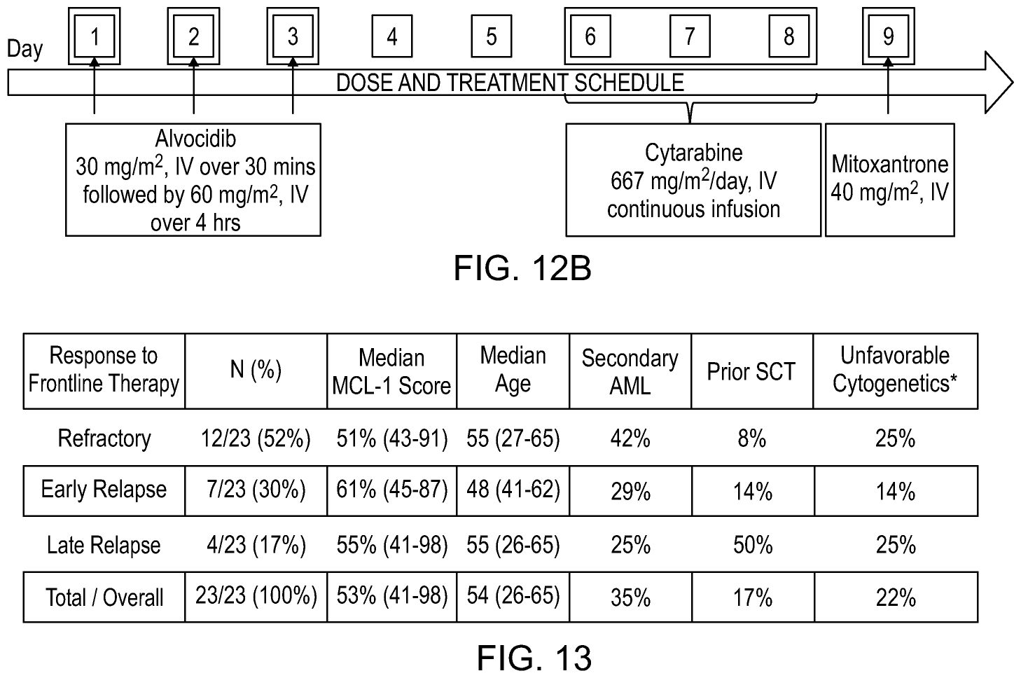

FIGS. 12A and 12B are diagrams summarizing the design of the Zella 201 clinical trial (CT.gov identifier: NTC02520011). The clinical trial is a phase 2, biomarker-driven, clinical trial to compare alvocidib administered together with cytabine and mitoxantrone (ACM) with cytarabine and mitoxantrone alone in patients with MCL-1-dependent, relapsed, or refractory AML. FIG. 12A provides an overview of Stage 1 and Stage 2 of the Zella 201 clinical trial. FIG. 12B provides an overview of a dose and treatment schedule for the Zella 201 clinical trial.

FIG. 13 lists patient characteristics for the Zella 201 clinical trial. Refractory: Persistent disease or CR duration <90 days. Early relapse: CR duration, 90 days—1 year. Late relapse: CR duration, 1 year—2 years. Two patients are excluded from the list: a suicide attempt resulting in death (n=1); failure to complete treatment (n=1). N=23 evaluable patients in stage 1. “*” indicates that unfavorable cytogenetics correspond to Southwest Oncology Group (SWOG) cytogenetic risk group.

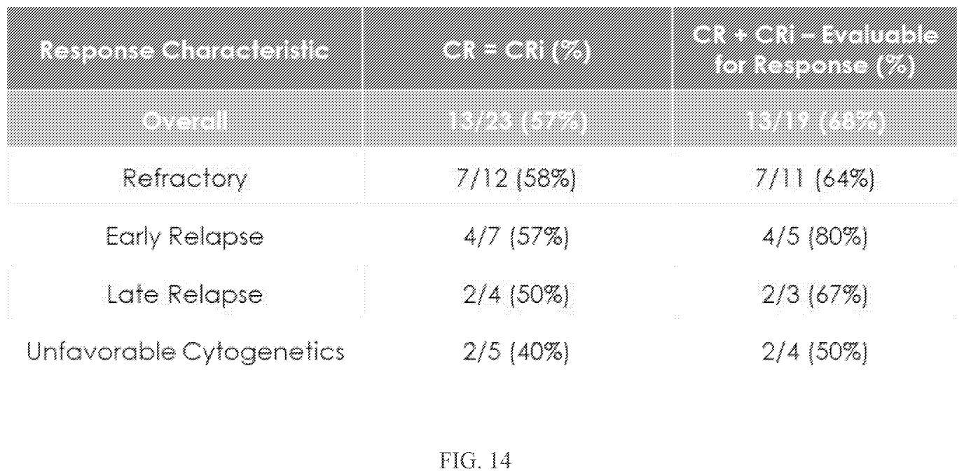

FIG. 14 lists response characteristics for Stage 1 of the Zella 201 clinical trial. CR=10; CRi=3; ORβ=61% (1 patient received PR). 4 early deaths before response assessment. 43% patients proceeded to allogeneic stem cell transplant (10 total). Median OS=11.2 mos (95% CI [3.0, 16.8]). Stage 1 met criteria of CR rate to proceed to Stage 2. OS trends compared favorably with historical controls with a median OS of 11.1 months.

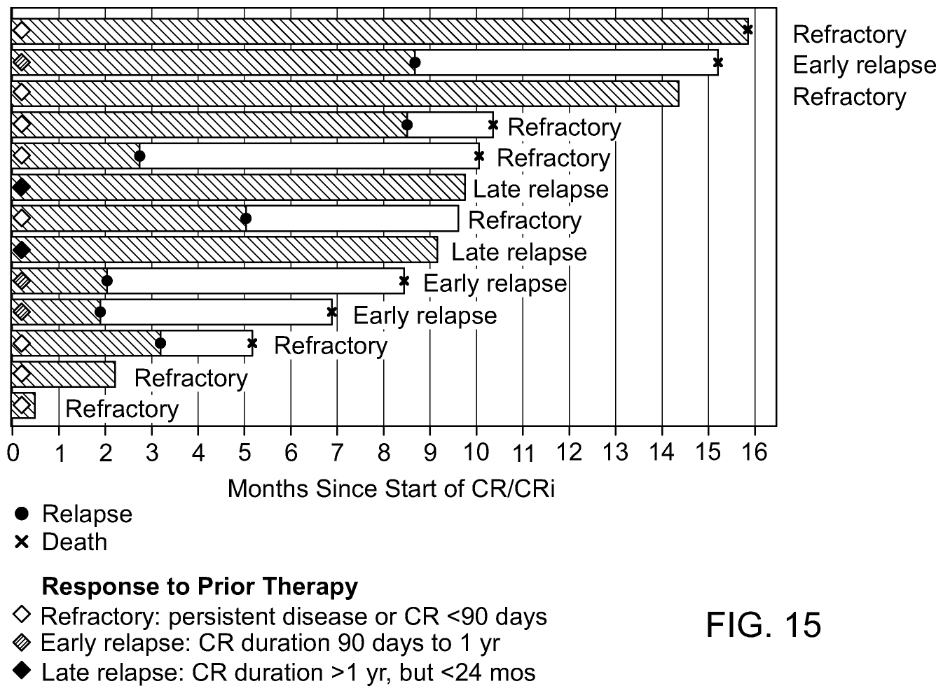

FIG. 15 is a graph summarizing responder (CR/CRi) results from the Zella 201 clinical trial. Median duration of response was 8.5 mos (95% CI [2.2, 15.9]). Overall CR rate was 57%.

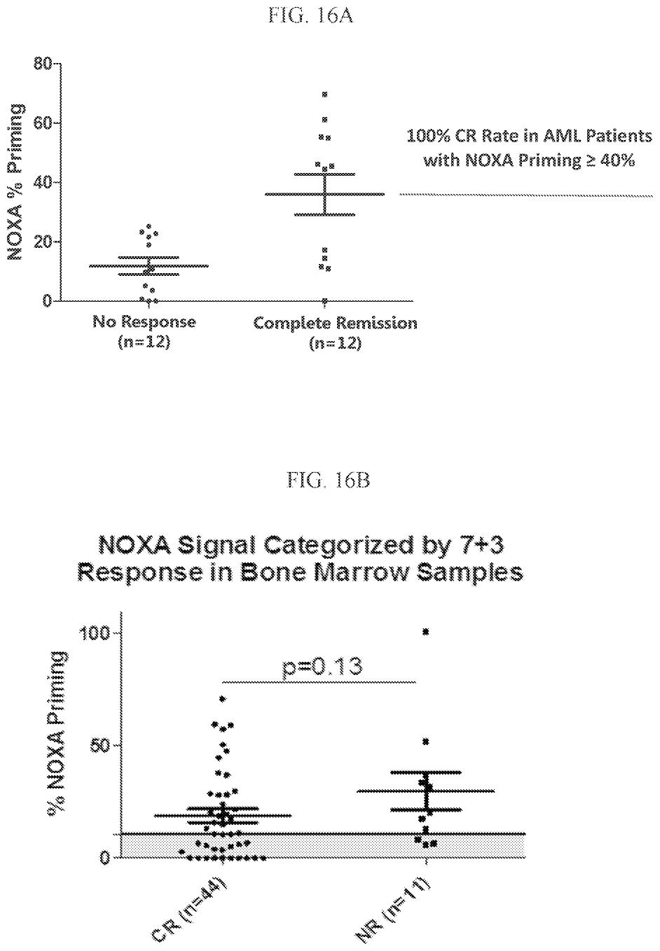

Furthermore, high NOXA Priming (MCL-1 dependence) is predictive of alvocidib sensitivity in AML patients. FIGS. 16A and 16B show that NOXA priming is predictive of response to alvocidib, while NOXA priming is not predictive of response to 7+3 chemotherapy. FIG. 16A is a box-plot showing that high NOXA priming (MCL-1 dependence) is predictive of alvocidib sensitivity in acute myeloid leukemia (AML) patients. The box-plot plots NOXA priming in complete remission and no response patient bone marrow samples taken from patients prior to being treated with alvocidib. Patients having pre-treatment bone marrow samples with NOXA priming greater than or equal to 40% had a 100% complete recovery rate. FIG. 16B is a box-plot showing that there is no correlation of response to NOXA-primed (MCL-1 dependent) patients to treatment with 7+3 chemotherapy (CR=complete remission; NR=no response). The box-plot plots NOXA priming in complete remission and no response patient bone marrow samples taken from patients prior to being treated with 7+3 chemotherapy.

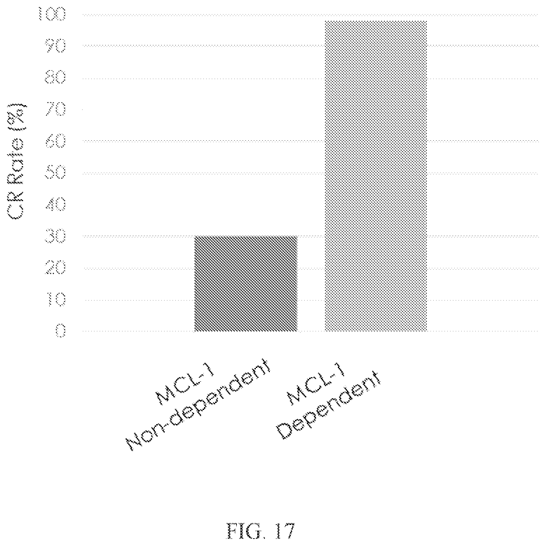

FIG. 17 is a bar graph demonstrating that MCL-1 dependence is a biomarker for ACM response and that MCL-1 dependence is predictive of alvocidib sensitivity in AML patients. The bar graph plots CR rate (%) for MCL-1 non-dependent (NOXA <40%) and MCL-1 dependent (NOXA priming >40%) patients. MCL-1 dependence did not predict response for 7+3 chemotherapy. MCL-1 Dependence was determined using CR and NR pre-treatment bone marrow samples from AML patients treated with an ACM regimen. ACM=alvocidib, cytarabine, mitoxantrone; NR=no response; CR=complete remission.

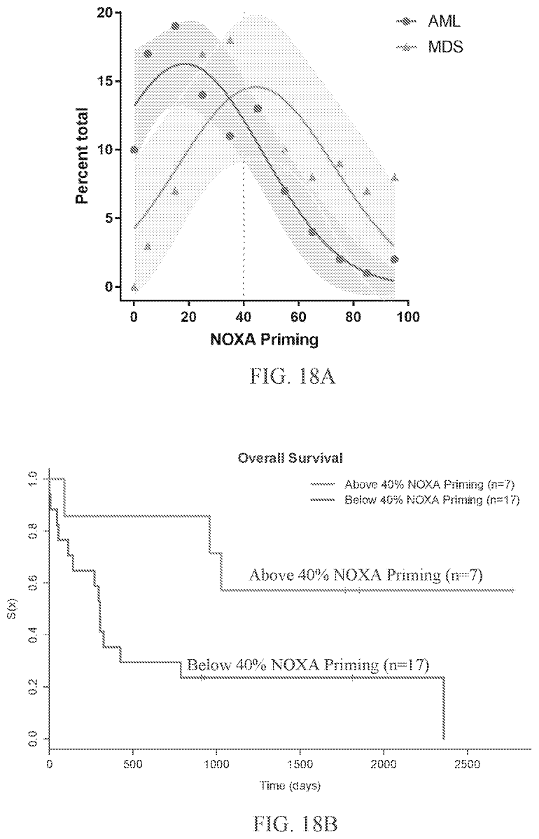

MDS patients are more highly NOXA primed (MCL-1 dependent). NOXA priming above 40% is considered highly NOXA primed. FIGS. 18A and 18B demonstrate that myelodysplastic syndrome (MDS) patients developing AML (secondary AML) are more likely to be responsive to alvocidib treatment than non-MDS AML patients. FIG. 18A is a Histogram and gaussian curve fit of AML and MDS patient sample NOXA priming results (95% CI in shaded areas). FIG. 18B shows that MDS patients are more highly NOXA primed (MCL-1 dependent) than a general AML patient population. Shaded areas represent 95% confidence interval. FIG. 18B is a plot showing that patients with NOXA priming (MCL-1 dependence) greater than or equal to 40% demonstrated greater survival than patients with NOXA priming less than 40%.

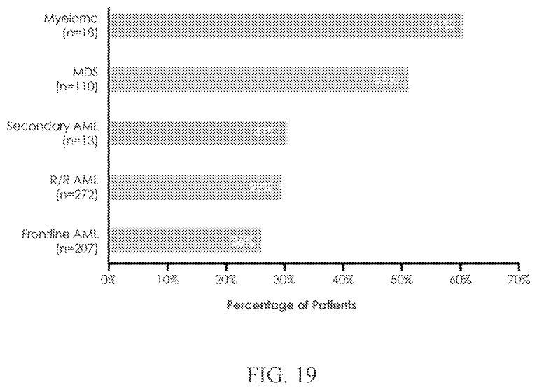

FIG. 19 is a bar graph supporting that MCL-1 dependence is prevalent in AML, MDS, and secondary AML (prior MDS) patients. FIG. 19 graphs the percentage of population estimated to be MCL-1 dependent, as determined by BH3 profiling. MCL-1 dependency is defined as induction of apoptosis in 40% or greater in a target cell population when exposed to an MCL-antagonist (NOXA memetic).

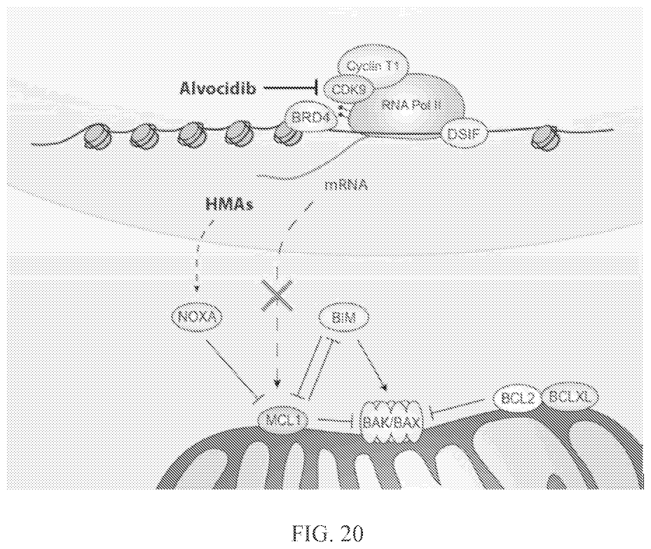

Without wishing to be bound by theory, it is hypothesized that a synergy mechanism exists between alvocidib and hypomethylating agents (HMAs), e.g., azacytidine and decitabine. As shown in FIG. 20, HMAs (e.g., azacytidine and decitabine) induce re-expression of key proteins, including NOXA. As described below, alvocidib, a CDK9 inhibitor, suppresses MCL-1 expression through RNA Pol II. Increased NOXA expression may increase sensitivity to MCL-1 loss. Thus, alvocidib and HMA, in combination, may have a synergistic effect on cancer cells, e.g., in myelodysplastic syndrome (MDS).

### Methods of the Disclosure

In one aspect, the present disclosure provides a method of treating MDS in a patient in need thereof, comprising administering to the patient a therapeutically effective amount of a HMA (e.g., azacitidine, or a prodrug thereof, or a pharmaceutically acceptable salt of the foregoing; or decitabine, or a prodrug thereof, or a pharmaceutically acceptable salt of the foregoing) and a therapeutically effective amount of alvocidib, or a prodrug thereof, or a pharmaceutically acceptable salt of the foregoing, wherein the patient has previously untreated MDS; received fewer than six cycles of treatment with a hypomethylating agent; de novo MDS; and/or secondary MDS. For example, in some embodiments, the patient has previously untreated, de novo MDS. In some embodiments, the patient has de novo MDS and has received fewer than six cycles of treatment with a HMA. In some embodiments, the patient has secondary MDS which is previously untreated. In some embodiments, the patient has secondary MDS and has received fewer than six cycles of treatment with a HMA. In some embodiments, the patient has de novo MDS which is previously untreated or the patient has de novo MDS and has received fewer than six cycles of treatment with a HMA. In some embodiments, the patient has secondary MDS which is previously untreated or the patient has secondary MDS and has received fewer than six cycles of treatment with a HMA.

In another aspect, the present disclosure provides a method of treating MDS in a patient in need thereof, comprising administering to the patient a therapeutically effective amount of azacitidine, or a prodrug thereof, or a pharmaceutically acceptable salt of the foregoing; and a therapeutically effective amount of alvocidib, or a prodrug thereof, or a pharmaceutically acceptable salt of the foregoing. The azacitidine, or a prodrug thereof, or a pharmaceutically acceptable salt of the foregoing, is administered on days 1, 2, 3, 4, 5, 6 and 7, or on days 1, 2, 3, 4, 5, 8 and 9 of a 28-day treatment cycle. The alvocidib, or a prodrug thereof, or a pharmaceutically acceptable salt of the foregoing, is administered on day 10 of the 28-day treatment cycle.

In yet another aspect, the present disclosure provides a method of treating MDS in a patient in need thereof, comprising administering to the patient a therapeutically effective amount of decitabine, or a prodrug thereof, or a pharmaceutically acceptable salt of the foregoing; and a therapeutically effective amount of alvocidib, or a prodrug thereof, or a pharmaceutically acceptable salt of the foregoing. The decitabine, or a prodrug thereof, or a pharmaceutically acceptable salt of the foregoing, is administered on days 1, 2, 3, 4 and 5 of a 28-day treatment cycle. The alvocidib, or a prodrug thereof, or a pharmaceutically acceptable salt of the foregoing, is administered on day 8 of the 28-day treatment cycle.

In another aspect, the present disclosure provides a method of treating MDS in a patient with previously untreated MDS comprising administering to the patient a therapeutically effective amount of a HMA and a therapeutically effective amount of alvocidib.

In another aspect, the present disclosure provides a method of treating MDS in a patient who received fewer than six cycles of treatment with a HMA comprising administering to the patient a therapeutically effective amount of an HMA and a therapeutically effective amount of alvocidib.

In another aspect, the present disclosure provides a method of treating MDS in a patient with de novo MDS who has not received a previous MDS treatment comprising administering to the patient a therapeutically effective amount of a HMA and a therapeutically effective amount of alvocidib.

In one embodiment, the patient is not eligible for intensive induction chemotherapy or a stem cell transplant.

NCCN, ELN and ESMO have issued guidelines for transplant eligibility in MDS. NCCN's guidelines state that transplant eligibility principles include patients having fit performance status, age, and having a donor. The HCT-CI can be used to evaluate the significance of comorbidities on survival outcomes of patients. See https://www.nccn.org/professionals/physician_gls/pdf/mds.pdf. ELN's guidelines state that the assessment of individual risk enables the identification of fit patients with a poor prognosis who are candidates for up-front intensive treatments, primarily allogeneic stem cell transplantation. Comorbidity predicts posttransplantation outcome. HCT-CI is an instrument that captures pretransplantation comorbidities and can be used in predicting posttransplantation outcomes and stratifying patients with MDS. See https://ashpublications.org/blood/article-lookup/doi/10.1182/blood-2013-03-492884. ESMO's guidelines state that the major obstacle to alloSCT is the fact that most MDS patients are above the age of 70 years. Co-morbidity, age, IPSS and IPSS-R score, cytogenetics, conditioning regimen and donor selection are predictors of post-transplant outcome and should be taken into account carefully during the decision process. All patients diagnosed with higher-risk MDS aged <65-70 years (although particularly ‘fit’ patients aged >70 years may sometimes be considered) should be evaluated for alloSCT eligibility. HLA-identical (or single antigen mismatched) siblings or matched unrelated individuals should be considered as suitable donors. See https://www.annalsofoncology.org/article/S0923-7534(19)34080-3/pdf.

In some embodiments, a subject having MDS is transplant ineligible or transplant eligible (e.g., transplant ineligible) according to NCCN guidelines. In some embodiments, a subject having MDS is transplant eligible or transplant ineligible (e.g., transplant ineligible) according to ELN guidelines. In some embodiments, a subject having MDS is transplant ineligible or transplant eligible (e.g., transplant ineligible) according to ESMO guidelines.

Lindsley, R. C., et al., Blood 26 Feb. 2015, Volume 125, No. 9, 1367-76 identified a genomic/genetic signature specific for secondary AML. Lindsley et al. showed that the presence of a mutation in any one or more of SRSF2, SF3B1, U2AF1, ZRSR2, ASXL1, EZH2, BCOR and STAG2 was >95% specific for secondary AML. This so-called genetic signature of secondary AML is shared by therapy-related AML and elderly de novo AML populations, and is associated with a subset of AML patients with worse clinical outcomes, including a lower CR rate, more frequent re-induction and decreased, event-free survival. Lindsley et al.

Mutations in RUNX1 have also been observed in AML patients. For example, mutations in RUNX1 and/or ASXL1, particularly when unaccompanied by favorable-risk genetics (e.g., t(8;21)(q22;q22.1); RUNX1-RUNX1Ti inv(16)(p13.1q22) or t(16;16)(p13.1;q22); CBFB-MYH11; mutated NPM1 without FLT3-ITD or with FLT3-ITD^{low}; biallelic mutated CEBPA), are associated with adverse-risk AML according to ELN risk stratification guidelines in recognition of their independent association with adverse risk. RUNX1 mutations, for example, are associated with poor prognosis, and ASXL1 mutations with inferior survival. Dohner, H., et al., Blood 26 Jan. 2017; Vol. 129; No. 4; 424-447.

Without wishing to be bound by theory, and considering that about 30% of MDS cases progress to AML, it is hypothesized that the presence of a mutation in any one or more of RUNX1, SRSF2, SF3B1, U2AF1, ZRSR2, ASXL1, EZH2, BCOR and STAG2 may also be a genetic signature associated with MDS and, in particular, clinical outcomes in MDS.

Accordingly, in some embodiments, the patient (e.g., a patient having an MDS described herein) has a mutation (e.g., one or more mutations) in one or more (e.g., one, at least two, two, at least three, three, at least four, four, at least five, five, at least six, six, at least seven, seven, at least eight, eight, nine) of RUNX1, SRSF2, SF3B1, U2AF1, ZRSR2, ASXL1, EZH2, BCOR and STAG2. In some embodiments, a patient has a mutation (e.g., one or more mutations) in NPM1. In some embodiments, a patient has a mutation (e.g., one or more mutations) in one or more (e.g., one, at least two, two, at least three, three, at least four, four, at least five, five, at least six, six, at least seven, seven, at least eight, eight, at least nine, nine, ten) of RUNX1, SRSF2, SF3B1, U2AF1, ZRSR2, ASXL1, EZH2, BCOR, STAG2 and NPM1. Non-limiting examples of mutation patterns include mutation(s) in RUNX1; SRSF2; SF3B1; U2AF1; ZRSR2; ASXL1; EZH2; BCOR; STAG2; NPM1; SRSF2 and BCOR; IDH2, SRSF2 and BCOR; NPM1; NPM1, IDH1 and NRAS; FLT3; CEBPA; ASXL1 and TET2; RUNX1, IDH1, SRSF2 and BCOR; RUNX1, SRSF2 and BCOR; RUNX1, IDH2 and SRSF2; RUNX1 and SRSF2; TP53; U2AF1 and BCOR; DNMT3A, IDH1 and NPM1; NPM1 and DNMT3A; NPM1 and TET2; NPM1, DNMT3A and NRAS; NPM1, FLT3, CEBPA, DNMT3A; ASXL1, RUNX1, EZH2, IDH2 and NRAS; ASXL1, RUNX1 and EZH2; FL T3, ASXL1, RUNX1 and BCOR; and ASXL1, RUNX1 and BCOR.

In some embodiments of any of the methods described herein, the method further comprises determining whether a subject has one or more mutations in one or more of RUNX1, SRSF2, SF3B1, U2AF1, ZRSR2, ASXL1, EZH2, BCOR and STAG2; and administering a therapeutically effective amount of alvocidib, or a prodrug thereof, or a pharmaceutically acceptable salt of the foregoing to the subject, if the subject is determined to have one or more mutations in one or more of RUNX1, SRSF2, SF3B1, U2AF1, ZRSR2, ASXL1, EZH2, BCOR and STAG2.

In another aspect, the present disclosure provides a method of treating MDS in a patient with secondary MDS who has not received a previous MDS treatment comprising administering to the patient a therapeutically effective amount of a hypomethylating agent (HMA) and a therapeutically effective amount of alvocidib.

In one embodiment, the patient is not eligible for intensive induction chemotherapy or a stem cell transplant.

In one embodiment, the patient has one or more mutations in one or more of RUNX1, SRSF2, SF3B1, U2AF1, ZRSR2, ASXL1, EZH2, BCOR and STAG2.

In another embodiment, the MDS is selected from the group consisting of refractory anemia (RA), refractory anemia with ringed sideroblasts (RARS), refractory anemia with excess blasts (RAEB), refractory anemia with excess blasts in transformation (RAEB-T), and chronic myelomonocytic leukemia (CMML).

The IPSS-R provides a standard for predicting prognosis in adult patients with untreated MDS. Several features of the IPSS-R are highlighted in Tables A, B and C. The IPSS-R is described in Greenberg, Tuechler, Schanz et al., Revised International Prognostic Scoring System (IPSS-R) for Myelodysplastic Syndrome, Blood 120: 2454, 2012, the entire contents of which are incorporated herein by reference.

TABLE A
IPSS-R Cytogenetic Risk Groups

| Cytogenetic | |
|---|---|
| prognostic | |
| subgroups | Cytogenetic abnormalities |
| Very good | −Y, del(11q) |
| Good | Normal, del(5q), del(12p), del(20q), double including |
| | del(5q) |
| Intermediate | del(7q), +8, +19, (17q), any other single or double |
| | independent clones |
| Poor | −7, inv(3)/t(3q)/del(3q), double including −7/del(7c), |
| | Complex: 3 abnormalities |
| Very poor | Complex: >3 abnormalities |

TABLE B
IPSS-R Prognostic Score Values

| Prognostic variable | 0 | 0.5 | 1 | 1. [symbol] | 2 | 3 | 4 |
|---|---|---|---|---|---|---|---|
| Cytogenetics | Very good | — | Good | — | Intermediate | Poor | very poor |
| BM blast, % | ≤2 | — | >2%-<5% | — | 6%-10% | >10% | — |
| Hemoglobin | ≥10 | — | 8-<10 | <8 | — | — | — |
| Platelets | ≥100 | 50-<100 | <50 | — | — | — | — |
| ANC | ≥0.8 | <0.8 | — | — | — | — | — |

— indicates not applicable. [symbol]
indicates data missing or illegible when filed

TABLE C
IPSS-R Prognostic Risk Categories/Scores

| Risk category | Risk score |
|---|---|
| Very low | ≤1.5 |
| Low | >1.5-3 |
| Intermediate | >3-4.5 |
| High | >4.5- [symbol] |
| Very high | [symbol] |

[symbol]

indicates data missing or illegible when filed

In some embodiments, the MDS is intermediate-, high- or very high-risk, e.g., according to the IPSS-R prognostic risk categories/scores. In some embodiments, the MDS is intermediate-risk, e.g., according to the IPSS-R prognostic risk categories/scores. In some embodiments, the MDS is high- or very high-risk, e.g., according to the IPSS-R prognostic risk categories/scores.

In another embodiment, the MDS is selected from an intermediate-1 Revised International Prognostic Scoring System (IPSS-R) group, an intermediate-2 IPSS-R group, and a high IPSS-R group.

In another embodiment, the HMA and the alvocidib are administered simultaneously.

In another embodiment, the HMA and the alvocidib are administered sequentially.

In another embodiment, the HMA is administered first, followed by administration of alvocidib.

In one embodiment, the HMA and/or alvocidib may be administered as a prodrug, e.g., as a biologically inactive or less active compound that may be metabolized to produce the HMA and/or alvocidib drug.

In one embodiment, the HMA is administered as a prodrug.

In another embodiment, the alvocidib is administered as a prodrug.

In another embodiment, the alvocidib prodrug is an alvocidib phosphate prodrug.

In another embodiment, the alvocidib phosphate prodrug is a compound having the structure

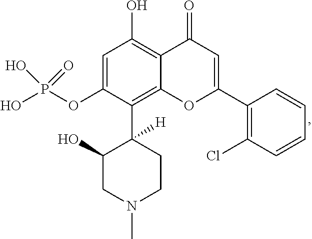 [SMILES: CN1CC[C@H](c2c(OP(=O)(O)O)cc(O)c3c(=O)cc(-c4ccccc4Cl)oc23)[C@H](O)C1]

or a pharmaceutically acceptable salt thereof.

In one embodiment of any of the above methods, the method further comprising administering the HMA and alvocidib in combination with another active agent.

In another embodiment, the HMA is administered in combination with a cytidine deaminase inhibitor.

In one embodiment, the HMA may be administered systemically. The term “systemic” as used herein includes parenteral, topical, transdermal, oral, by inhalation/pulmonary, rectal, nasal, buccal, and sublingual administration. The term “parenteral” as used herein includes subcutaneous, intradermal, intravenous, intramuscular, intracranial, and intraperitoneal administration.

In another embodiment, the HMA is administered intravenously or by subcutaneous injection.

In another embodiment, the HMA is selected from azacitidine and decitabine.

### Azacitidine Administration

In another embodiment, the HMA is azacitidine, or a prodrug thereof, or a pharmaceutically acceptable salt of the foregoing. For example, in some embodiments, the HMA is azacitidine, or a pharmaceutically acceptable salt thereof (e.g., azacitidine). In other embodiments, the HMA is a prodrug of azacitidine (e.g., a phosphate prodrug of azacitidine), or a pharmaceutically acceptable salt thereof.

In another embodiment, the azacitidine is administered as an azacitidine phosphate prodrug, or a pharmaceutically acceptable salt thereof (e.g., azacitidine phosphate prodrug). By way of a non-limiting example, one azacitidine prodrug suitable for use in the present methods is disclosed in WO2011/153374, which is hereby incorporated by reference in its entirety.

In another embodiment, the azacitidine phosphate prodrug has the formula

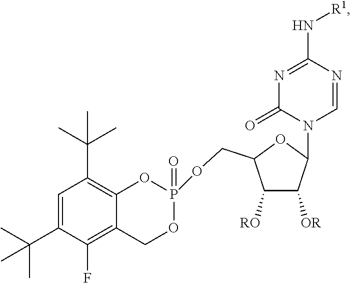

where R and R¹ are independently H or CO₂(C₁-C₆ alkyl).

In another embodiment, R is H at each occurrence and R¹ is selected from H and CO₂(C₅ alkyl).

In another embodiment, the azacitidine is 2′,3′,5′-triacetyl-5-azacitidine, or a pharmaceutically acceptable salt thereof (e.g., 2′,3′,5′-triacetyl-5-azacitidine).

In another embodiment, the azacitidine, or a prodrug thereof, or a pharmaceutically acceptable salt of the foregoing (e.g., azacitidine) is administered orally.

In another embodiment, the azacitidine is administered as CC-486 composition. CC-486 composition is an azacitidine composition formulated for oral administration.

In another embodiment, the azacitidine, or a prodrug thereof, or a pharmaceutically acceptable salt of the foregoing (e.g., azacitidine) is administered as an intravenous infusion. The intravenous infusion may be administered instantaneously or over time. In one embodiment, the intravenous infusion may be administered over a period of from about 1 minute to about 6 hours, or from about 2 minutes to about 4 hours, or from about 5 minute to about 2 hours, or from about 5 minutes to about 100 minutes, or from about 10 minute to about 40 minutes, or about 10 minutes, or about 15 minutes, or about 20 minutes, or about 25 minutes, or about 30 minutes, or about 35 minutes, or about 40 minutes.

In another embodiment, the intravenous infusion of azacitidine, or a prodrug thereof, or a pharmaceutically acceptable salt of the foregoing (e.g., azacitidine) is over from about 5 to about 100 minutes.

In another embodiment, the intravenous infusion of azacitidine, or a prodrug thereof, or a pharmaceutically acceptable salt of the foregoing (e.g., azacitidine) is over from about 10 to about 40 minutes.

In another embodiment, the azacitidine, or a prodrug thereof, or a pharmaceutically acceptable salt of the foregoing (e.g., azacitidine) is administered subcutaneously.

In one embodiment, the azacitidine, or a prodrug thereof, or a pharmaceutically acceptable salt of the foregoing (e.g., azacitidine) is administered daily on consecutive days. In one embodiment, the azacitidine, or a prodrug thereof, or a pharmaceutically acceptable salt of the foregoing (e.g., azacitidine) is administered consecutively for at least 2 days, or 3 days, or 4 days, or 5 days, or 6 days, or 7 days.

In another embodiment, the azacitidine, or a prodrug thereof, or a pharmaceutically acceptable salt of the foregoing (e.g., azacitidine) is administered consecutively for 7 days (e.g., on days 1, 2, 3, 4, 5, 6 and 7 of a treatment cycle, such as a 28-day treatment cycle).

In one embodiment, the administration of azacitidine, or a prodrug thereof, or a pharmaceutically acceptable salt of the foregoing (e.g., azacitidine) may be interrupted by a drug holiday. By way of an example, azacitidine, or a pharmaceutically acceptable salt thereof (e.g., azacitidine) may be administered for 3-5 days, followed by 1-3 azacitidine-free days, then followed by administration of azacitidine, or a pharmaceutically acceptable salt thereof (e.g., azacitidine) for 2-4 days.

In another embodiment, the azacitidine, or a pharmaceutically acceptable salt thereof (e.g., azacitidine) is administered once daily for 5 days (e.g., on days 1, 2, 3, 4 and 5 of a treatment cycle, such as a 28-day treatment cycle), followed by once-daily administration of azacitidine, or a pharmaceutically acceptable salt thereof (e.g., azacitidine), for 2 days (e.g., on days 8 and 9 of the treatment cycle, such as the 28-day treatment cycle). In another embodiment, the azacitidine, or a pharmaceutically acceptable salt thereof (e.g., azacitidine) is administered once daily for 5 days (e.g., on days 1, 2, 3, 4 and 5 of a treatment cycle, such as a 28-day treatment cycle), followed by 2 azacitidine-free days (e.g., on days 6 and 7 of the treatment cycle, such as the 28-day treatment cycle), then followed by once daily administration of azacitidine, or a pharmaceutically acceptable salt thereof (e.g., azacitidine) for 2 days (e.g., on days 8 and 9 of the treatment cycle, such as the 28-day treatment cycle).

In another embodiment, the azacitidine, or a prodrug thereof, or a pharmaceutically acceptable salt of the foregoing (e.g., azacitidine) is administered at a dosage of about 10 mg/m² to about 90 mg/m².

In another embodiment, the azacitidine, or a prodrug thereof, or a pharmaceutically acceptable salt of the foregoing (e.g., azacitidine), is administered at a dosage lower than about 90 mg/m² and subsequently escalated to the dosage of about 90 mg/m².

In another embodiment, the azacitidine, or a prodrug thereof, or a pharmaceutically acceptable salt of the foregoing (e.g., azacitidine), is administered at a dosage of about 75 mg/m².

In another embodiment, the alvocidib, or a prodrug thereof, or a pharmaceutically acceptable salt of the foregoing (e.g., alvocidib), is administered on day 10 from the start of the azacitidine, or a prodrug thereof, or a pharmaceutically acceptable salt of the foregoing (e.g., azacitidine), administration.

In another embodiment, the alvocidib, or a prodrug thereof, or a pharmaceutically acceptable salt of the foregoing (e.g., alvocidib), is administered as an intravenous infusion.

The intravenous infusion may be administered instantaneously or over time. In one embodiment, the intravenous infusion may be administered over a period of from about 1 minute to about 6 hours, or from about 5 minutes to about 4 hours, or from about 10 minutes to about 2 hours, or from about 30 minutes to about 90 minutes, or from about 45 minutes to about 75 minutes, or about 30 minutes, or about 45 minutes, or about 60 minutes, or about 75 minutes, or about 90 minutes.

In another embodiment, the intravenous infusion is over from about 20 to about 120 minutes.

In another embodiment, the intravenous infusion over about 1 hour.

In another embodiment, the alvocidib, or a prodrug thereof, or a pharmaceutically acceptable salt of the foregoing (e.g., alvocidib), is administered at a dosage of about 90 mg/m².

Thus, in some embodiments, from about 50 mg/m² to about 125 mg/m², preferably, about 75 mg/m², azacitidine, or a prodrug thereof, or a pharmaceutically acceptable salt of the foregoing (e.g., azacitidine, or a pharmaceutically acceptable salt thereof), can be administered to a subject once per day by intravenous infusion of about 10 minutes to about 40 minutes in duration or by subcutaneous injection for from five to ten days, preferably for 5 days or 7 days.

In some embodiments, azacitidine, or a prodrug thereof, or a pharmaceutically acceptable salt of the foregoing (e.g., azacitidine, or a pharmaceutically acceptable salt thereof), is administered daily for 7 consecutive days (e.g., on days 1-7 of a treatment cycle, such as a 28-day treatment cycle). When a 7-day treatment schedule is employed, days 8 and 9 are typically azacitidine-free days and, in some embodiments, are drug holidays.

Alternatively, azacitidine, or a prodrug thereof, or a pharmaceutically acceptable salt thereof (e.g., azacitidine, or a pharmaceutically acceptable salt thereof), can be administered according to a 5-2-2 regimen, in which azacitidine, or a prodrug thereof, or a pharmaceutically acceptable salt thereof, is administered once daily for 5 consecutive days (e.g., on days 1-5 of a treatment cycle, such as a 28-day treatment cycle) and once daily for 2 days (e.g., on days 8 and 9 of the treatment cycle, such as the 28-day treatment cycle). When a 5-2-2 treatment schedule is employed, days 6 and 7 are typically azacitidine-free days and, in some embodiments, are drug holidays.

In some embodiments, from about 150 mg to about 350 mg, preferably, about 200 mg or 300 mg, azacitidine, or a pharmaceutically acceptable salt thereof (e.g., CC-486), is administered to a subject once per day orally, e.g., for 7, 14 or 21 days (e.g., on days 1-7, 1-14 or 1-21, respectively, of a 21-day or 28-day cycle). In some embodiments, an effective amount or a therapeutically effective amount of CC-486 is administered to a subject once per day orally, e.g., for 7, 14 or 21 days (e.g., on days 1-7, 1-14 or 1-21, respectively, of a 21-day or 28-day cycle).

When alvocidib, or a pharmaceutically acceptable salt thereof, is used in combination with azacitidine, the alvocidib, or a pharmaceutically acceptable salt thereof, can be administered once during the treatment cycle (e.g., on day 10 of the treatment cycle) using any of the dosages and dosing schedules for alvocidib, or a pharmaceutically acceptable salt thereof, described herein (e.g., by intravenous infusion of about one hour in duration in a dose of about 90 mg/m² alvocidib, or a pharmaceutically acceptable salt thereof).

### Decitabine Administration

In another embodiment, the HMA is decitabine, or a prodrug thereof, or a pharmaceutically acceptable salt of the foregoing. In some embodiments, the HMA is decitabine, or a pharmaceutically acceptable salt thereof (e.g., decitabine). In other embodiments, the HMA is a prodrug of decitabine, or a pharmaceutically acceptable salt thereof.

In another embodiment, the decitabine, or a prodrug thereof, or a pharmaceutically acceptable salt of the foregoing (e.g., decitabine), is administered in combination with cedazuridine, for example, about 100 mg of cedazuridine, as in ASTX727.

In another embodiment, the decitabine, or a prodrug thereof, or a pharmaceutically acceptable salt of the foregoing (e.g., decitabine), is administered as ASTX727.

In another embodiment, the decitabine, or a prodrug thereof, or a pharmaceutically acceptable salt of the foregoing (e.g., decitabine), is administered as an intravenous infusion.

The intravenous infusion may be administered instantaneously or over time. In one embodiment, the intravenous infusion may be administered over a period of from about 1 minute to about 6 hours, or from about 5 minutes to about 4 hours, or from about 10 minutes to about 2 hours, or from about 30 minutes to about 90 minutes, or from about 45 minutes to about 75 minutes, or about 30 minutes, or about 45 minutes, or about 60 minutes, or about 75 minutes, or about 90 minutes.

In another embodiment, the intravenous infusion is over from about 20 to about 120 minutes.

In another embodiment, the intravenous infusion over about 1 hour.

In one embodiment, the decitabine, or a prodrug thereof, or a pharmaceutically acceptable salt of the foregoing (e.g., decitabine), is administered daily on consecutive days. In one embodiment, the decitabine, or a prodrug thereof, or a pharmaceutically acceptable salt of the foregoing (e.g., decitabine), is administered consecutively for at least 2 days, or 3 days, or 4 days, or 5 days, or 6 days, or 7 days.

In another embodiment, the decitabine, or a prodrug thereof, or a pharmaceutically acceptable salt of the foregoing (e.g., decitabine), is administered daily for 5 days (e.g., on days 1-5 of a treatment cycle, such as a 28-day treatment cycle).

In another embodiment, the alvocidib is administered on day 8 from the start of the decitabine administration.

Thus, in some embodiments, from about 15 mg/m² to about 50 mg/m², preferably, about 20 mg/m², decitabine, or a pharmaceutically acceptable salt thereof, can be administered to a subject once per day by intravenous infusion of about 1 hour in duration for from three to ten (e.g., consecutive) days. In some embodiments, from about 15 mg to about 50 mg, preferably, about 35 mg, decitabine, or a pharmaceutically acceptable salt thereof, can be administered to a subject once per day orally for from three to ten (e.g., consecutive) days (e.g., 5 days, such as 5 consecutive days). In some embodiments, about 35 mg decitabine, or a pharmaceutically acceptable salt thereof, and about 100 mg cedazuridine, or a pharmaceutically acceptable salt thereof, (e.g., ASTX727) can be administered to a subject once per day orally for from three to ten (e.g., consecutive) days (e.g., 5 days, such as 5 consecutive days). In some embodiments, decitabine is administered daily for 5 days (e.g., on days 1-5 of a treatment cycle, such as a 28-day treatment cycle). When decitabine, or a pharmaceutically acceptable salt thereof, is administered on days 1-5 of a treatment cycle, days 6 and 7 are typically decitabine-free days and, in some embodiments, are drug holidays.

When alvocidib, or a pharmaceutically acceptable salt thereof, is used in combination with decitabine, or a prodrug thereof, or a pharmaceutically acceptable salt thereof, the alvocidib, or a pharmaceutically acceptable salt thereof, can be administered once during the treatment cycle (e.g., on day 8 of the treatment cycle) using any of the dosages and dosing schedules for alvocidib, or a pharmaceutically acceptable salt described herein (e.g., by intravenous bolus of about 30 minutes in duration in a dose of about 30 mg/m² alvocidib, or a pharmaceutically acceptable salt thereof, followed by an intravenous infusion of about 4 hours in duration in a dose of about 60 mg/m² alvocidib, or a pharmaceutically acceptable salt thereof, by intravenous infusion of about one hour in duration in a dose of about 90 mg/m² alvocidib, or a pharmaceutically acceptable salt thereof).

### Alvocidib Administration

In one embodiment, alvocidib, or a pharmaceutically acceptable salt thereof, is administered. Typically, alvocidib, or a pharmaceutically acceptable salt is administered intravenously. In some embodiments, alvocidib, or a pharmaceutically acceptable salt thereof, is administered by intravenous bolus of from about 10 minutes to about 60 minutes, from about 15 minutes to about 45 minutes or about 30 minutes in duration. When alvocidib, or a pharmaceutically acceptable salt thereof, is administered by intravenous bolus, typically from about 5 mg/m² to about 50 mg/m², from about 20 mg/m² to about 30 mg/m², from about 25 mg/m² to about 35 mg/m² or from about 25 mg/m² to about 60 mg/m² (e.g., about 25 mg/m², about 30 mg/m², about 50 mg/m²) is administered in the bolus. In some embodiments, about 30 mg/m² alvocidib, or a pharmaceutically acceptable salt thereof, is administered by intravenous bolus, for example, once daily for three consecutive days, for example, on a 28-day cycle. In some embodiments, from about 20 mg/m² to about 30 mg/m² (e.g., about 20 mg/m², about 30 mg/m²) alvocidib, or a pharmaceutically acceptable salt thereof, is administered by intravenous bolus, for example, once per treatment cycle, for example, on day 8 or day 10 of the treatment cycle. In some embodiments, about 50 mg/m² alvocidib, or a pharmaceutically acceptable salt thereof, is administered by intravenous bolus, for example, once weekly (e.g., once weekly for three consecutive weeks, for example, on a 28-day cycle). In some embodiments, about 25 mg/m² alvocidib, or a pharmaceutically acceptable salt thereof, is administered once by intravenous bolus, for example, on day 1 of a 28-day treatment cycle, and 50 mg/m² alvocidib, or a pharmaceutically acceptable salt thereof, is administered once by intravenous bolus, for example, on day 15 of the 28-day treatment cycle. In some embodiments, about 25 mg/m² alvocidib, or a pharmaceutically acceptable salt thereof, is administered once by intravenous bolus, for example, on day 1 of a 28-day treatment cycle, and 50 mg/m² alvocidib, or a pharmaceutically acceptable salt thereof, is administered twice by intravenous bolus, for example, on days 8 and 15 of the 28-day treatment cycle.

In some embodiments, alvocidib, or a pharmaceutically acceptable salt thereof, is administered by intravenous infusion of from about 3 hours to about 5 hours, from about 3.5 hours to about 4.5 hours or about 4 hours (e.g., ±30 minutes) in duration. In some embodiments, alvocidib, or a pharmaceutically acceptable salt thereof, is administered by infusion of from about 30 minutes to about one hour in duration. In some embodiments, alvocidib, or a pharmaceutically acceptable salt thereof, is administered by infusion of about one hour in duration (e.g., one hour±15 minutes). When alvocidib, or a pharmaceutically acceptable salt thereof, is administered by intravenous infusion, typically from about 10 mg/m² to about 100 mg/m², from about 25 mg/m² to about 90 mg/m², from about 10 mg/m² to about 65 mg/m², from about 30 mg/m² to about 60 mg/m², from about 75 mg/m² to about 100 mg/m² (e.g., about 75 mg/m², about 90 mg/m²) or from about 50 mg/m² to about 75 mg/m² (e.g., about 25 mg/m², about 30 mg/m², about 40 mg/m², about 50 mg/m², about 60 mg/m²) is administered in the infusion. In some embodiments, about 60 mg/m² alvocidib, or a pharmaceutically acceptable salt thereof, is administered by intravenous infusion, for example, once daily for three consecutive days, for example, on a 28-day cycle. In some embodiments, from about 30 mg/m² to about 60 mg/m² (e.g., about 30 mg/m², about 40 mg/m², about 50 mg/m², about 60 mg/m²) alvocidib, or a pharmaceutically acceptable salt thereof, is administered by intravenous infusion, for example, once per treatment cycle. In some embodiments, from about 80 mg/m² to about 100 mg/m² (e.g., about 90 mg/m²) alvocidib, or a pharmaceutically acceptable salt thereof, is administered by intravenous infusion, for example, once per treatment cycle. In some embodiments, from about 25 mg/m² to about 90 mg/m² alvocidib, or a pharmaceutically acceptable salt thereof, is administered by intravenous infusion, for example, weekly or on day 8 when administration of alvocidib, or a pharmaceutically acceptable salt thereof, follows administration of a hypomethylating agent, such as azacitidine or decitabine, or a pharmaceutically acceptable salt of the foregoing.

In some embodiments, alvocidib, or a pharmaceutically acceptable salt thereof, is administered by intravenous bolus, for example, as described herein, and intravenous infusion, for example, as described herein. When alvocidib, or a pharmaceutically acceptable salt thereof, is administered by intravenous bolus and intravenous infusion, the bolus typically precedes the intravenous infusion. In some embodiments, an intravenous infusion of alvocidib, or a pharmaceutically acceptable salt thereof, is initiated within about one hour (e.g., within about 45 minutes, within about 30 minutes) of completion of the bolus of alvocidib, or a pharmaceutically acceptable salt thereof. In some embodiments, an intravenous infusion of alvocidib, or a pharmaceutically acceptable salt thereof, is initiated about 30 minutes after completion of a bolus of alvocidib, or a pharmaceutically acceptable salt thereof. In some embodiments, about 30 mg/m² alvocidib, or a pharmaceutically acceptable salt thereof, is administered by intravenous bolus, and then about 60 mg/m² alvocidib, or a pharmaceutically acceptable salt thereof, is administered by intravenous infusion. Administration of a so-called hybrid dose of alvocidib, or a pharmaceutically acceptable salt thereof (a dose administered by intravenous bolus and intravenous infusion), can occur according to any one of the treatment cycles and/or dosing schedules described herein.

In some embodiments, from about 10 mg/m² to about 100 mg/m², from about 25 mg/m² to about 60 mg/m², from about 75 mg/m² to about 100 mg/m², about 50 mg/m², about 75 mg/m² or about 90 mg/m² alvocidib, or a pharmaceutically acceptable salt thereof, is administered to a patient per day.

In other embodiments, a prodrug of alvocidib, or a pharmaceutically acceptable salt thereof, is administered. Examples of prodrugs of alvocidib suitable for use in the methods of the present disclosure include those described hereinabove, and include the crystalline forms of prodrugs of alvocidib described hereinabove.

Prodrugs of alvocidib (e.g., a compound of Structural Formula I, Ia or Ib), or a pharmaceutically acceptable salt thereof, can be administered once per day or more than once per day, for example, twice per day.

In some embodiments, a prodrug of alvocidib (e.g., a compound of Structural Formula I, Ia or Ib, such as Form B of the compound of Structural Formula Ib), or a pharmaceutically acceptable salt thereof, is administered on the first 14 days of a 21-day treatment cycle. When this treatment schedule is employed, the prodrug of alvocidib, or a pharmaceutically acceptable salt thereof, is typically not administered on days 15 to 21 of the 21-day treatment cycle, which are alvocidib-free days and, in some embodiments, are drug holidays. In other embodiments a prodrug of alvocidib (e.g., a compound of Structural Formula I, Ia or Ib, such as Form B of the compound of Structural Formula Ib), or a pharmaceutically acceptable salt thereof, is administered on the first 21 days of a 28-day treatment cycle. When this treatment schedule is employed, the prodrug of alvocidib, or a pharmaceutically acceptable salt thereof, is typically not administered on days 22 to 28 of the 28-day treatment cycle, which are alvocidib-free days and, in some embodiments, are drug holidays.

A prodrug of alvocidib (e.g., a compound of Structural Formula I, Ia or Ib, such as Form B of the compound of Structural Formula Ib), or a pharmaceutically acceptable salt thereof, is effective over a wide dosage range. For example, in the treatment of adult humans, dosages from about 0.01 mg to about 1000 mg, from about 0.5 mg to about 100 mg, from about 0.5 mg to about 100 mg, from about 1 mg to about 50 mg per day, and from about 5 mg to about 40 mg per day are examples of dosages that are used in some embodiments. In particular embodiments, the dosage ranges from about 1 mg to about 60 mg (e.g., from about 5 mg to about 60 mg, from about 10 mg to about 60 mg, from about 5 mg to about 50 mg, from about 10 mg to about 30 mg, from about 10 mg to about 50 mg, from about 20 to about 50 mg, from about 25 mg to about 45 mg) per day. In other embodiments, the dosage is from about 1 mg to about 30 mg per day, e.g., about 1 mg, about 2 mg, about 4 mg, about 8 mg, about 12 mg, about 16 mg, about 20 mg, about 22 mg, about 24 mg, about 26 mg, about 28 mg, about 30 mg or about 32 mg per day (e.g., administered QD, administered BID). In other embodiments, the dosage is from about 1 mg to about 30 mg, e.g., about 1 mg, about 2 mg, about 4 mg, about 6 mg, about 8 mg, about 11 mg, about 12 mg, about 16 mg, about 20 mg, about 22 mg, about 24 mg, about 26 mg, about 28 mg or about 30 mg, administered BID. The exact dosage will depend, for example, upon the route of administration, the form in which the prodrug is administered, the subject to be treated, the body weight of the subject to be treated, and the preference and experience of the attending physician.

In one embodiment, the alvocidib, or a pharmaceutically acceptable salt thereof (e.g., alvocidib), is administered by an intravenous infusion.

In one embodiment, the alvocidib, or a pharmaceutically acceptable salt thereof (e.g., alvocidib), is administered as a bolus followed by an intravenous infusion.

In one embodiment, the bolus is over about 1 to about 60 minutes, or about 5 to about 50 minutes, or about 10 to about 40 minutes, or about 10 minutes, or about 20 minutes, or about 30 minutes, or about 40 minutes, or about 50 minutes.

In one embodiment, the bolus is over about 10 to about 40 minutes.

In another embodiment, the bolus is over about 30 minutes.

In one embodiment, the alvocidib, or a pharmaceutically acceptable salt thereof (e.g., alvocidib), is administered by an intravenous infusion without a bolus.

In one embodiment, the intravenous infusion may be administered over a period of from about 1 minute to about 12 hours, or from about 5 minutes to about 10 hours, or from about 10 minutes to about 8 hours, or from about 30 minutes to about 6 hours, or from about 1 hour to about 5 hours, or about 1 hour, or about 2 hours, or about 3 hours, or about 4 hours, or about 5 hours, or about 6 hours.

In another embodiment, the intravenous infusion is over from about 30 minutes to about 6 hours.

In another embodiment, the intravenous infusion is over about 4 hours.

In another embodiment, the alvocidib, or a pharmaceutically acceptable salt thereof (e.g., alvocidib), is administered as a bolus at a dosage of about 20 mg/m² followed by an intravenous infusion at a dosage of about 10 mg/m² to about 60 mg/m².

In another embodiment, the alvocidib, or a pharmaceutically acceptable salt thereof (e.g., alvocidib), is administered at an overall dosage of about 20 mg/m² to about 100 mg/m².

In another embodiment, the alvocidib, or a pharmaceutically acceptable salt thereof (e.g., alvocidib), is administered as an intravenous infusion.

In another embodiment, the intravenous infusion is over about 1 hour.

In another embodiment, the alvocidib, or a pharmaceutically acceptable salt thereof (e.g., alvocidib), is administered at a dosage of about 90 mg/m².

In another embodiment, the decitabine, or a pharmaceutically acceptable salt thereof (e.g., decitabine) is administered at a daily dosage of about 10 mg/m² to about 30 mg/m².

In another embodiment, the decitabine, or a pharmaceutically acceptable salt thereof (e.g., decitabine) is administered at a daily dosage of about 20 mg/m². Compositions, Combinations and Kits

The therapeutic agents described herein (e.g., alvocidib, or a prodrug thereof, or a pharmaceutically acceptable salt of the foregoing; azacitidine, or a prodrug thereof, or a pharmaceutically acceptable salt of the foregoing; decitabine, or a prodrug thereof, or a pharmaceutically acceptable salt of the foregoing, etc.) can be administered in pure form or in an appropriate pharmaceutical composition comprising one or more therapeutic agents (e.g., a pharmaceutical combination), and one or more pharmaceutically acceptable carriers.

A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012)).

Typically, pharmaceutically acceptable carriers are sterile. The pharmaceutical composition can be formulated for particular routes of administration such as oral administration, parenteral administration (e.g., intravenous administration) and rectal administration, etc. In addition, the pharmaceutical composition can be made up in a solid form (including, without limitation, capsules, tablets, pills, granules, powders or suppositories), or in a liquid form (including, without limitation, solutions, suspensions or emulsions). The pharmaceutical compositions can be subjected to conventional pharmaceutical operations, such as sterilization, and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone; d) disintegrants, e.g., starches, agar, alginic acid or its sodium salt, or effervescent mixtures; and
e) absorbents, colorants, flavors and sweeteners.
   Tablets may be either film-coated or enteric-coated according to methods known in the art.

Suitable compositions for oral administration include a therapeutic agent described herein (e.g., a compound of Structural Formula I, Ia or Ib, or a pharmaceutically acceptable salt of the foregoing) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

A pharmaceutical composition for use in the present methods may be in the form of a liquid, for example, an elixir, syrup, solution, emulsion or suspension. The liquid may be for oral administration, such as for compositions comprising a prodrug of alvocidib, azacitidine and/or decitabine, or a pharmaceutically acceptable salt of any of the foregoing, or for delivery by injection, such as form compositions comprising alvocidib, or a pharmaceutically acceptable salt thereof, for example. When intended for oral administration, pharmaceutical compositions contain, for example in addition to the therapeutic compound(s), one or more of a sweetening agent, preservative, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably, physiological saline, Ringer's solution, isotonic sodium chloride, fixed oils such as synthetic mono- and diglycerides which may serve as the solvent or suspending medium, polyethylene glycols, glycerin, propylene glycol and other solvents; antibacterial agents such as benzyl alcohol and methyl paraben; antioxidants such as ascorbic acid and sodium bisulfite; chelating agents such as ethylenediaminetetraacetic acid; buffers such as acetates, citrates and phosphates; and agents for the adjustment of tonicity, such as sodium chloride and dextrose. A parenteral preparation can be enclosed in ampoules, disposable syringes or multiple dose vials made of glass or plastic. Physiological saline is a preferred adjuvant. An injectable pharmaceutical composition is preferably sterile. In embodiments, the pharmaceutical composition is formulated for injection. In some embodiments, the pharmaceutical composition is formulated for bolus injection. In embodiments, the pharmaceutical composition is formulated for infusion.

Certain injectable compositions comprise a therapeutic agent described herein (e.g., alvocidib, or a pharmaceutically acceptable salt thereof, azacitidine, or a pharmaceutically acceptable salt thereof; decitabine, or a pharmaceutically acceptable salt thereof) in the form of an aqueous isotonic solution or suspension, and certain suppositories comprising a therapeutic agent described herein are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include a therapeutic agent described herein with a suitable carrier. Carriers suitable for transdermal delivery include absorbable pharmacologically acceptable solvents to assist passage through the skin of the host. For example, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

Suitable compositions comprising a therapeutic agent described herein for topical application, e.g., to the skin and eyes, include aqueous solutions, suspensions, ointments, creams, gels or sprayable formulations, e.g., for delivery by aerosol or the like. Such topical delivery systems will, in particular, be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein, a topical application may also pertain to an inhalation or to an intranasal application. A composition suitable for inhalation or intranasal administration may be conveniently delivered in the form of a dry powder (either alone, as a mixture, for example a dry blend with lactose, or a mixed component particle, for example, with phospholipids) from a dry powder inhaler, or an aerosol spray presentation from a pressurised container, pump, spray, atomizer or nebuliser, with or without the use of a suitable propellant.

A therapeutic agent described herein can also be provided in anhydrous pharmaceutical compositions and dosage forms, since water may facilitate the degradation of certain compounds. Anhydrous pharmaceutical compositions and dosage forms can be prepared using anhydrous or low moisture-containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Pharmaceutical compositions and dosage forms can also comprise one or more agents that reduce the rate by which a therapeutic agent will decompose. Such agents, which are referred to herein as “stabilizers,” include, but are not limited to, antioxidants such as ascorbic acid, pH buffers, or salt buffers, etc.

A pharmaceutical composition used in certain embodiments of the disclosure may be prepared by methodology well known in the pharmaceutical art. For example, a pharmaceutical composition intended to be administered by injection can be prepared by combining one or more of the therapeutic agents with sterile, distilled water so as to form a solution. In some embodiments, pharmaceutical composition(s) for administration according to methods of the disclosure take the form of a liquid where the therapeutic agents are present in solution, in suspension, or both. In some embodiments, when a therapeutic agent is administered as a solution or suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of the therapeutic agents. The term “solubilizing agent” generally includes agents that result in formation of a micellar solution or a true solution of the agent. Certain acceptable nonionic surfactants, for example polysorbate 80, are useful as solubilizing agents, as are ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

A surfactant may be added to facilitate the formation of a homogeneous solution or suspension. Surfactants are compounds that non-covalently interact with a therapeutic agent so as to facilitate dissolution or homogeneous suspension. In embodiments, a pharmaceutical composition includes one or more surfactants to enhance physical stability. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

A pharmaceutical composition for use in embodiments of the disclosure may include various materials that modify the physical form of a solid or liquid dosage unit. For example, the composition may include materials that form a coating shell around one or more of the therapeutic agents. The materials that form the coating shell are typically inert, and may be selected from, for example, sugar, shellac, and other enteric coating agents. Alternatively, the active ingredients may be encased in a gelatin capsule.

A pharmaceutical composition used in certain embodiments may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of the therapeutic agents may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

A therapeutic agent described herein is typically formulated into pharmaceutical dosage forms to provide an easily controllable dosage of the drug and to give the patient an elegant and easily handleable product. The dosage regimen will, of course, vary depending upon known factors, such as the pharmacodynamic characteristics of the particular therapeutic agent and its mode and route of administration; the species, age, sex, health, medical condition, and weight of the recipient; the nature and extent of the symptoms; the kind of concurrent treatment; the frequency of treatment; the route of administration; the renal and hepatic function of the patient; and the effect desired. Therapeutic agents described herein may be administered in a single daily dose, or the total daily dosage may be administered in divided doses, e.g., two, three, or four times daily.

Compositions for use in combination therapies will either be formulated together as a pharmaceutical combination, or provided for separate administration (e.g., associated in a kit). Accordingly, a further embodiment is a pharmaceutical combination comprising two or more therapeutic agents described herein. A pharmaceutical combination can further comprise one or more pharmaceutically acceptable carriers, such as one or more of the pharmaceutically acceptable carriers described herein.

A pharmaceutical composition can be in a unit dosage containing from about 1 to about 1000 mg of active ingredient(s) for a subject of from about 50 to about 70 kg, or from about 1 to about 500 mg, from about 1 to about 250 mg, from about 1 to about 150 mg, from about 0.5 to about 100 mg, or from about 1 to about 50 mg of active ingredient(s) for a subject of from about 50 to about 70 kg. The effective and/or therapeutically effective dosage of a therapeutic agent/pharmaceutical composition is dependent on the species of the subject, the body weight, age and individual condition of the subject, and the disorder or disease or the severity thereof being treated. A physician, clinician or veterinarian of ordinary skill can readily determine the effective and/or therapeutically effective amount of each of the active ingredients necessary to prevent or treat the progress of the disorder or disease.

In some embodiments, the concentration of one or more therapeutic agents provided in a pharmaceutical composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.00010% w/w, w/v or v/v.

In some embodiments, the concentration of one or more therapeutic agents provided in a pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%1, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

Another aspect is a kit comprising two or more, separate therapeutic agents (e.g., two or more, separate pharmaceutical compositions). In one embodiment, the kit comprises a therapeutically effective amount of each therapeutic agent (e.g., each pharmaceutical composition). For example, in some embodiments, a kit comprises alvocidib, or a prodrug thereof (e.g., a compound of Structural Formula Ia or Ib), or a pharmaceutically acceptable salt of the foregoing, and an HMA (e.g., azacitidine, or a prodrug thereof, or a pharmaceutically acceptable salt of the foregoing; decitabine, or a prodrug thereof, or a pharmaceutically acceptable salt of the foregoing).

The kit of the present disclosure may be used for administering different dosage forms, for example, oral and parenteral, for administering the separate compositions at different dosage intervals, or for titrating the separate compositions against one another.

To assist compliance, a kit typically comprises directions for administration. The written instructions may include instructions regarding dosage, method of administration, order and timing of administration, and the like. The written instructions can be in the form of printed instructions provided within the kit, or the written instructions can be printed on a portion of the container housing the kit. Written instructions may be in the form of a sheet, pamphlet, brochure, CD-ROM, or computer-readable device, or can provide directions to locate instructions at a remote location, such as a website. The written instructions may be in English and/or in a national or regional language.

Kits can further comprise one or more syringes, ampules, vials, tubes, tubing, facemask, a needleless fluid transfer device, an injection cap, sponges, sterile adhesive strips, Chloraprep, gloves, and the like. Variations in contents of any of the kits described herein can be made. In various embodiments, the contents of the kit are provided in a compact container.

In some embodiments, pharmaceutical compositions of the disclosure are presented in a pack or dispenser device that contains one or more unit dosage forms containing the active ingredient(s). The pack may, for example, comprise metal or plastic foil, such as a blister pack.

In embodiments, the kit (e.g., a pack or dispenser) may be accompanied by a notice associated with the container in a form prescribed by a governmental agency regulating the manufacture, use or sale of pharmaceuticals, which notice is reflective of approval by the agency of the form of the compositions or of human or veterinary administration, in addition to instructions for administration. Such notice, for example, may be of the labeling approved by the U.S. Food and Drug Administration for prescription drugs or of an approved product insert.

### Tumor Lysis Syndrome Prophylaxis

In another embodiment, the patient is further administered a tumor lysis syndrome prophylaxis.

In another embodiment, the tumor lysis syndrome prophylaxis comprises intravenous hydration with, e.g., an aqueous salt. In one embodiment, the aqueous salt is aqueous NaCl. In one embodiment, the aqueous NaCl has a concentration of about 0.05% to about 5%, or about 0.1% to about 2.5%, or about 0.25% to about 1%, or about 0.4% to about 0.6%, or about 0.4%, or about 0.45%, or about 0.5% aqueous NaCl.

In another embodiment, the tumor lysis syndrome prophylaxis comprises intravenous hydration with a 0.45% aqueous NaCl.

In another embodiment, the tumor lysis syndrome prophylaxis comprises administering one or more of allopurinol, an oral phosphate binder, replacement of fluid losses, and an anti-diarrheal medication.

In another embodiment, the tumor lysis syndrome prophylaxis is administered prior to first HMA dose.

In another embodiment, the tumor lysis syndrome prophylaxis is administered prior to first alvocidib dose.

### Patient Populations

In one embodiment, the patient is an adult, i.e., the patient is 18 years old or greater.

In another embodiment, the patient is a child under 18 years of age.

In another embodiment, the patient has an Eastern Cooperative Oncology Group (ECOG) Performance Status (PS) score which is less than or equal to 2 according to the below Table 1.

TABLE 1
ECOG PERFORMANCE STATUS SCALE
ECOG PERFORMANCE STATUS*

| Grade | ECOG |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all selfcare but unable to carry |
| | out any work activities. Up and about more than 50% of |
| | waking hours |
| 3 | Capable of only limited selfcare, confined to bed or chair |
| | more than 50% of waking hours |
| 4 | Completely disabled. Cannot carry on any selfcare. Totally |
| | confined to bed or chair |
| 5 | Dead |

Oken M M, Creech R H, Tormey D C, et al. Toxicity And Response Criteria Of The Eastern Cooperative Oncology Group. Am J Clin Oncol 1982; 5: 649-655. Available at: http://www.ecog.org/general/perf_stat.html

In another embodiment, the patient has a life expectancy of greater than or equal to: 1 month, or 2 months, or 3 months, or 4 months, or 6 months, or 9 months, or 12 months.

In another embodiment, the patient has a life expectancy of greater than or equal to 3 months.

In another embodiment, the patient has one or more mutations in one or more of RUNX1, SRSF2, SF3B1, U2AF1, ZRSR2, ASXL1, EZH2, BCOR and STAG2.

In another embodiment, the patient meets the following criteria based on laboratory data:
a) wherein the patient's serum creatinine is less than or equal to 1.8 times the upper limit of the normal (ULN) range;
b) wherein the patient's total bilirubin is less than or equal to 2 times the ULN range, and
c) wherein the patient's aspartate transaminase (AST) and alanine transaminase (ALT) are less than or equal to 3 times the ULN range.

In another embodiment, the patient does not have a concomitant severe cardiovascular disease.

In another embodiment, the patient does not have a condition selected from New York Heart Association (NYHA) Functional Class III or IV heart disease (see Table 2), National Cancer Institute (NCI) Common Terminology Criteria for Adverse Events (CTCAE) v5.0 grade equal to or greater than 3 arrhythmia, angina pectoris, abnormal electrocardiogram findings, interstitial pneumonia, and pulmonary fibrosis.

TABLE 2
NEW YORK HEART ASSOCIATION CLASSIFICATION
OF HEART FAILURE

| NYHA | | | |
|---|---|---|---|
| Class | Definition | Limitation | Example |
| I | Ordinary | None | Can complete any activity |
| | physical activity | | requiring ≤7 mets: |
| | does not cause | | Carry 11 kg up 8 steps |
| | undue fatigue, | | Carry objects weighing 36 kg |
| | dyspnea, | | Shovel snow |
| | palpitation | | Spade soil |
| | | | Ski |
| | | | Play squash, handball or |
| | | | basketball |
| | | | Jog/walk 8 km/h |
| II | Ordinary | Slight | Can complete any activity |
| | physical activity | | requiring ≤5 mets: |
| | causes fatigue, | | Sexual intercourse without |
| | dyspnea, | | stopping |
| | palpitation, or | | Garden |
| | angina | | Roller skate |
| | | | Walk 7 km/h on level ground |
| | | | Climb one flight of stairs at a |
| | | | normal pace without symptoms |
| III | Comfortable at | Moderate | Can complete any activity |
| | rest; less than | | requiring ≤2 mets: |
| | ordinary | | Shower or dress without |
| | physical activity | | stopping |
| | causes fatigue, | | Strip and make bed |
| | dyspnea, | | Clean windows |
| | palpitation, or | | Play golf |
| | angina | | Walk 4 km/h |
| IV | Symptoms at | Severe | Cannot do or cannot complete |
| | rest; any | | any activity requiring ≥2 |
| | physical activity | | mets; cannot do any of the |
| | increases | | above activities |
| | discomfort | | |

Mets = metabolic equivalents
Reference: http://www.merck.com/mmpe/sec07/ch074/ch074a.html#CEGDEIFG

The National Cancer Institute Common Terminology Criteria for Adverse Events v5.0 (NCI CTCAE) criteria can be viewed electronically at the following Web site: https://ctep.cancer.gov/protocoldevelopment/electronic_applications/docs/CTCAE_v5_Quick_Reference_8.5x11.pdf.

In another embodiment, the patient has not had myocardial infarction within 6 months before the treatment.

In one embodiment, the patient does not have a concomitant malignancy.

In another embodiment, the patient does not have a concomitant malignancy requiring chemotherapy, or a concomitant malignancy for which the patient received chemotherapy within 6 months prior to treatment, with the proviso that the malignancy is not selected from basal and squamous cell carcinoma of the skin.

In another embodiment, the patient does not have an uncontrolled or uncontrollable infection, or a Grade equal to or greater than 3 infection according to NCI CTCAE v5.0.

In another embodiment, the patient does not have a dry tap on bone marrow aspiration.

In another embodiment, the patient does not have a concurrent autoimmune disease or a history of chronic or recurrent autoimmune disease.

In another embodiment, the patient does not require a long-term systemic steroid therapy greater than the equivalent of 20 mg of prednisone daily.

In another embodiment, the patient does not have another documented malignancy within the past year.

In another embodiment, the patient does not have Grade equal to or greater than 2 hemorrhage according to NCI CTCAE v5.0.

In another embodiment, the patient is not pregnant or breastfeeding.

In another embodiment, the patient has not previously received alvocidib or another cyclin-dependent kinase 9 (CDK9) inhibitor.

In another embodiment, the method further comprises determining a BH3 profile for the patient's tumor cell specimen.

### Biomarker Profile

In some embodiments of any of the methods described herein, the method further comprises assessing one or more biomarkers associated with MDS. Assessing includes measuring or determining the level of one or more biomarkers (e.g., presence or absence of a biomarker, upregulation or downregulation of a biomarker compared to an appropriate control) and determining mutational status and/or epigenetic mutational status of one or more biomarkers, and can be done at the level of DNA, RNA (e.g., mRNA) or protein. For example, MCL-1 dependency or MCL-1 mRNA expression can be assessed. NOXA methylation or NOXA mRNA expression can also or alternatively be assessed. LINE-1 methylation can be also or alternatively be assessed.

In some embodiments, the one or more biomarkers associated with MDS is selected from the group consisting of nucleic acids, proteins, lipids, and metabolites. For example, in some embodiments, the one or more biomarkers associated with MDS includes MCL-1 or MCL-1 mRNA. In some embodiments, the one or more biomarkers associated with MDS includes NOXA or NOXA mRNA. In some embodiments, the one or more biomarkers associated with MDS includes long interspersed element-1 (LINE-1).

In some embodiments, the one or more biomarkers associated with MDS includes one or more (e.g., one, at least two, two, at least three, three, at least four, four, at least five, five, at least six, six, at least seven, seven, at least eight, eight, nine) of RUNX1, SRSF2, SF3B1, U2AF1, ZRSR2, ASXL1, EZH2, BCOR and STAG2. Thus, in some embodiments of any of the methods described herein, the method further comprises determining whether a subject has one or more mutations in one or more of (e.g., one, at least two, two, at least three, three, at least four, four, at least five, five, at least six, six, at least seven, seven, at least eight, eight, nine) RUNX1, SRSF2, SF3B1, U2AF1, ZRSR2, ASXL1, EZH2, BCOR and STAG2; and administering a therapeutically effective amount of alvocidib, or a prodrug thereof, or a pharmaceutically acceptable salt of the foregoing to the subject, if the subject is determined to have one or more mutations in one or more of RUNX1, SRSF2, SF3B1, U2AF1, ZRSR2, ASXL1, EZH2, BCOR and STAG2. In some embodiments, the one or more biomarkers associated with MDS includes or further includes NPM1. Non-limiting examples of biomarkers associated with MDS include RUNX1; SRSF2; SF3B1; U2AF1; ZRSR2; ASXL1; EZH2; BCOR; STAG2; NPM1; SRSF2 and BCOR; IDH2, SRSF2 and BCOR; NPM1; NPM1, IDH1 and NRAS; FLT3; CEBPA; ASXL1 and TET2; RUNX1, IDH1, SRSF2 and BCOR; RUNX1, SRSF2 and BCOR; RUNX1, IDH2 and SRSF2; RUNX1 and SRSF2; TP53; U2AF1 and BCOR; DNMT3A, IDH1 and NPM1; NPM1 and DNMT3A; NPM1 and TET2; NPM1, DNMT3A and NRAS; NPM1, FLT3, CEBPA, DNMT3A; ASXL1, RUNX1, EZH2, IDH2 and NRAS; ASXL1, RUNX1 and EZH2; FLT3, ASXL1, RUNX1 and BCOR; and ASXL1, RUNX1 and BCOR.

Methods of conducting mutation analyses, e.g., to determine whether a subject has one or more mutations, are known in the art, and include next generation sequencing. For example, institutions use commercially available products and reagents to establish their own molecular pathology analytic processes. The combined cancer panel from one institution, for example, targets exonic and intronic sequences obtained from DNA purified from tumor (with or without normal DNA) using Custom Agilent SureSelect capture and Illumina HiSeq2500 sequencing. Samples have an average coverage of at least 500-fold, and at least 30-fold coverage of greater than 98% of coding sequences in the region of interest. These sequences are evaluated for single nucleotide variants, and small insertions and deletions. Actionable mutations are confirmed by an orthologous method. In addition, several companies, including Hematologics, Inc. and Foundation Medicine, provide commercial mutation analysis services. There are also many commercial products, including FoundationOne® Heme (available from Foundation Medicine, Cambridge, MA), for performing comprehensive genomic profiling.

In another embodiment, any of the above methods further comprise classifying the patient for likelihood of response to MDS treatment based on the patient's BH3 profile.

In another embodiment, any of the above methods further comprise assessment of BH3 and/or other biomarkers in peripheral blood and/or bone marrow aspirates.

In one embodiment, the preliminary prevalence of MCL-1 dependency in untreated MDS patients may be evaluated via BH3 profiling. In another embodiment, the effect of HMA treatment on modulating BH3 profiling results in peripheral blood may be determined. In another embodiment, the effect of alvocidib administration in sequence after HMA treatment on modulating BH3 profiling results in peripheral blood may be determined.

In another embodiment, the method further comprises measurement of an additional biomarker associated with MDS.

In another embodiment, the additional biomarker associated with MDS is selected from the group consisting of nucleic acids, proteins, lipids, and metabolites.

Analyses may include, but are not limited to, assessment of BH3 profiling by flow cytometry with an emphasis on MCL-1 dependence, evaluating genetic mutations, and other biomarkers associated with MDS. In another embodiment, the additional biomarker associated with MDS is MCL-1.

In another embodiment, the BH3 profile is determined by flow cytometry.

In another aspect, the present disclosure provides a method for determining a response to MDS treatment comprising administering a hypomethylating agent and alvocidib to a patient with previously untreated MDS, the method comprising determining a BH3 profile for the patient's tumor cell specimen, and classifying the patient for likelihood of response to MDS treatment.

In another embodiment, the method further comprises measurement of an additional biomarker associated with MDS.

In another embodiment, the additional biomarker is selected from the group consisting of nucleic acids, proteins, lipids, and metabolites.

In another embodiment, the additional biomarker is MCL-1.

In another embodiment, the BH3 profile is determined by flow cytometry.

In yet another aspect, the present disclosure provides a method of treating a patient with myelodysplastic syndrome (MDS) comprising administering to the patient a therapeutically effective amount of a hypomethylating agent (HMA) selected from azacitidine and decitabine and subsequently administering to the patient a therapeutically effective amount of alvocidib.

In another embodiment, the HMA is administered intravenously.

In another embodiment, the HMA is decitabine administered at a dose of about 10 mg/m² to about 30 mg/m² for from about 1 to about 3 hours, once to three times/day.

In another embodiment, the decitabine is administered once/day for 3 to 7 days.

In another embodiment, the decitabine is administered once/day for 5 days.

In another embodiment, the decitabine is administered at a dose of about 20 mg/m² in a one hour infusion.

In another embodiment, the alvocidib is administered at a rate of about 10 mg/m² to about 120 mg/m².

In another embodiment, the alvocidib is administered two days after the cessation of the decitabine administration.

In another embodiment, the alvocidib is administered two days after the cessation of the decitabine administration.

In another embodiment, a portion of the alvocidib is administered as a bolus dose of from about 10 mg/m² to about 50 mg/m² over a period of about 10 minutes to about 60 minutes.

In another embodiment, the bolus dose is administered over a period of about 30 minutes.

In another embodiment, the bolus dose is from about 20 mg/m² to about 30 mg/m².

In another embodiment, from about 10 mg/m² to about 60 mg/m² of alvocidib is administered intravenously over a period of about 2 hours to about 6 hours.

In another embodiment, the alvocidib is administered over a period of about 4 hours.

In another embodiment, the dose of the alvocidib is from about 20 mg/m² to about 60 mg/m².

In another embodiment, the alvocidib is administered intravenously at a dose of about 90 mg/m² over a period of about 20 minutes to about 120 minutes.

In another embodiment, the alvocidib is administered over a period of about 1 hour.

In another embodiment, the administration of the alvocidib by intravenous infusion is begun within about 30 minutes of the completion of the bolus dose.

In another embodiment, the HMA is azacitidine at a dose of about 30 to about 90 mg/m².

In another embodiment, the dose is about 75 mg/m² per day.

In another embodiment, the azacitidine is administered for seven days as an intravenous bolus injection over about 10 to about 40 minutes or subcutaneous injection.

In another embodiment, the alvocidib is administered intravenously two days after the cessation of azacitidine administration.

In another embodiment, the alvocidib is administered on day 10 after the commencement of azacitidine administration with no azacitidine administration on days 8 and 9.

In another embodiment, 90 mg/m² of the alvocidib is administered intravenously over a period of about 20 minutes to about 120 minutes.

In another embodiment, the alvocidib is administered over a period of about 1 hour.

In another embodiment, the azacitidine is administered at a dose of about 30 to about 90 mg/m²/day for five consecutive days, followed by azacitidine-free days 6 and 7, further followed by intravenous administration of azacitidine at a dose of about 30 to about 90 mg/m² on days 8 and 9, and further followed by intravenous administration of the alvocidib on day 10.

In another embodiment, the azacitidine is administered at a dose of about 75 mg/m²/day by intravenous bolus injection on days 1 to 5 and days 8 and 9, and wherein the alvocidib is administered at a dose of about 90 mg/m² over a period of about one hour by intravenous infusion on day 10.

In another embodiment, the treatment is repeated at least once.

In another embodiment, a treatment cycle comprises 28 days.

In another embodiment, the treatment cycle is repeated at least once.

In another embodiment, the treatment is repeated for at least 4 cycles.

In another embodiment, a treatment cycle comprises four to six weeks.

In another embodiment, the treatment is repeated for at least 4 cycles.

In another embodiment, the HMA is administered orally.

In another embodiment, the HMA is administered as a prodrug.

In another embodiment, the HMA is administered in combination with a cytidine deaminase inhibitor.

In another embodiment, the HMA is decitabine.

In another embodiment, the cytidine deaminase inhibitor is cedazuridine.

In another embodiment, the HMA is an azacitidine phosphate prodrug.

In another embodiment, wherein the azacitidine prodrug has the formula

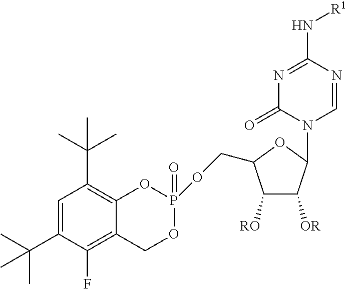

where R and R¹ are independently H or CO₂(C₁-C₆ alkyl).

In another embodiment, the HMA is the composition CC-486.

In another embodiment, the HMA is azacitidine administered as 2′,3′,5′-triacetyl-5-azacitidine.

### EMBODIMENTS

1. A method of treating myelodysplastic syndrome (MDS) in a patient with previously untreated MDS comprising administering to the patient a therapeutically effective amount of a hypomethylating agent (HMA) and a therapeutically effective amount of alvocidib.
2. A method of treating MDS in a patient who received fewer than six cycles of treatment with a hypomethylating agent (HMA) comprising administering to the patient a therapeutically effective amount of an HMA and a therapeutically effective amount of alvocidib.
3. A method of treating MDS in a patient with de novo MDS who has not received a previous MDS treatment comprising administering to the patient a therapeutically effective amount of a hypomethylating agent (HMA) and a therapeutically effective amount of alvocidib.
4. The method of embodiment Error! Reference source not found., wherein the patient is not eligible for intensive induction chemotherapy or a stem cell transplant.
5. A method of treating MDS in a patient with secondary MDS who has not received a previous MDS treatment comprising administering to the patient a therapeutically effective amount of a hypomethylating agent (HMA) and a therapeutically effective amount of alvocidib.
6. The method of embodiment Error! Reference source not found., wherein the patient is not eligible for intensive induction chemotherapy or a stem cell transplant.
7. The method of any of embodiments 1-Error! Reference source not found., wherein the MDS is selected from the group consisting of refractory anemia (RA), refractory anemia with ringed sideroblasts (RARS), refractory anemia with excess blasts (RAEB), refractory anemia with excess blasts in transformation (RAEB-T), and chronic myelomonocytic leukemia (CMML).
8. The method of any of embodiments 1-7, wherein the MDS is selected from an intermediate-1 Revised International Prognostic Scoring System (IPSS-R) group, an intermediate-2 IPSS-R group, and a high IPSS-R group.
9. The method of any of embodiments 1-Error! Reference source not found., wherein the HMA and the alvocidib are administered simultaneously.
10. The method of any of embodiments 1-Error! Reference source not found., wherein the HMA and the alvocidib are administered sequentially.
11. The method of embodiment Error! Reference source not found., wherein the HMA is administered first, followed by administration of alvocidib.
12. The method of embodiment Error! Reference source not found. or Error!Reference source not found., wherein the alvocidib is administered during a period of elevated NOXA expression following HMA administration.
13. The method of any of embodiments 1-Error! Reference source not found., wherein the HMA is administered as a prodrug.
14. The method of any of embodiments 1-9, wherein the alvocidib is administered as a prodrug.
15. The method of embodiment 26, wherein the alvocidib prodrug is an alvocidib phosphate prodrug.
16. The method of embodiment Error! Reference source not found., wherein the alvocidib phosphate prodrug is a compound having the structure

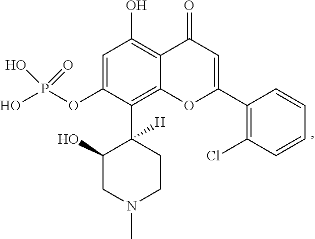 [SMILES: CN1CC[C@H](c2c(OP(=O)(O)O)cc(O)c3c(=O)cc(-c4ccccc4Cl)oc23)[C@H](O)C1]

or a pharmaceutically acceptable salt thereof.
17. The method of any of embodiments 1-Error! Reference source not found., wherein the HMA is administered in combination with a cytidine deaminase inhibitor.
18. The method of any of embodiments 1-45, wherein the HMA is administered intravenously or by subcutaneous injection.
19. The method of any of embodiments 1-Error! Reference source not found., wherein the HMA is selected from azacitidine and decitabine.
20. The method of any of embodiments 1-Error! Reference source not found., wherein the HMA is azacitidine.
21. The method of embodiment Error! Reference source not found., wherein the azacitidine is administered as an azacitidine phosphate prodrug.
22. The method of embodiment 0, wherein the azacitidine phosphate prodrug has the formula

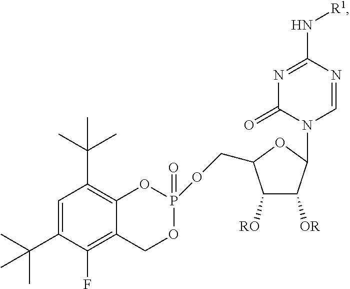

where R and R¹ are independently H or CO₂ (C₁-C₆ alkyl).
23. The method of embodiment Error! Reference source not found., wherein R is H at each occurrence and R¹ is selected from H and CO₂(C₅ alkyl).
24. The method of embodiment 0, wherein the azacitidine is 2′,3′,5′-triacetyl-5-azacitidine.
25. The method of embodiment Error! Reference source not found., wherein the azacitidine is administered orally.
26. The method of embodiment Error! Reference source not found., wherein the azacitidine is administered as CC-486 composition.
27. The method of embodiment Error! Reference source not found., wherein the azacitidine is administered as an intravenous infusion.
28. The method of embodiment 23, wherein the intravenous infusion is over from about 5 to about 100 minutes.
29. The method of embodiment Error! Reference source not found., wherein the intravenous infusion is over from about 10 to about 40 minutes.
30. The method of embodiment Error! Reference source not found., wherein the azacitidine is administered subcutaneously.
31. The method of any of embodiments Error! Reference source not found.-Error!Reference source not found., wherein the azacitidine is administered consecutively for 7 days.
32. The method of any of embodiments Error! Reference source not found.-Error!Reference source not found., wherein the azacitidine is administered once daily for 5 days, followed by 2 azacitidine-free days, then followed by once daily administration of azacitidine for 2 days.
33. The method of any of embodiments Error! Reference source not found.-Error!Reference source not found., wherein the azacitidine is administered at a dosage of about 10 mg/m² to about 90 mg/m².
34. The method of embodiment Error! Reference source not found., wherein the azacitidine is administered at a dosage lower than about 90 mg/m² and subsequently escalated to the dosage of about 90 mg/m².
35. The method of embodiment Error! Reference source not found., wherein the azacitidine is administered at a dosage of about 75 mg/m².
36. The method of any of embodiments Error! Reference source not found.-Error!Reference source not found., wherein the alvocidib is administered on day 10 from the start of the azacitidine administration.
37. The method of embodiments Error! Reference source not found.-Error! Reference source not found., wherein the alvocidib is administered during a period of elevated NOXA expression following azacitidine administration.
38. The method of embodiment Error! Reference source not found., wherein the alvocidib is administered as an intravenous infusion.
39. The method of embodiment 45, wherein the intravenous infusion is over from about 20 to about 120 minutes.
40. The method of embodiment Error! Reference source not found., wherein the intravenous infusion over about 1 hour.
41. The method of any of embodiments Error! Reference source not found.-Error!Reference source not found., wherein the alvocidib is administered at a dosage of about 90 mg/m².
42. The method of any of embodiments 1-Error! Reference source not found., wherein the HMA is decitabine.
43. The method of embodiment Error! Reference source not found., wherein the decitabine is administered in combination with cedazuridine.
44. The method of embodiment Error! Reference source not found., wherein the decitabine is administered as an intravenous infusion.
45. The method of embodiment Error! Reference source not found., wherein the intravenous infusion is over from about 20 to about 120 minutes.
46. The method of embodiment Error! Reference source not found., wherein the intravenous infusion over about 1 hour.
47. The method of any of embodiments Error! Reference source not found.-Error!Reference source not found., wherein the decitabine is administered daily for 5 days.
48. The method of embodiment Error! Reference source not found., wherein the alvocidib is administered on day 8 from the start of the decitabine administration.
49. The method of embodiments Error! Reference source not found.-Error! Reference source not found., wherein the alvocidib is administered during a period of elevated NOXA expression following decitabine administration.
50. The method of embodiment Error! Reference source not found., wherein the alvocidib is administered as a bolus followed by an intravenous infusion.
51. The method of embodiment Error! Reference source not found., wherein the bolus is over about 10 to about 40 minutes.
52. The method of embodiment Error! Reference source not found., wherein the bolus is over about 30 minutes.
53. The method of embodiment Error! Reference source not found., wherein the intravenous infusion is over from about 30 minutes to about 6 hours.
54. The method of embodiment Error! Reference source not found., wherein the intravenous infusion is over about 4 hours.
55. The method of any of embodiments Error! Reference source not found. to Error!Reference source not found., wherein the alvocidib is administered as a bolus at a dosage of about 20 mg/m² followed by an intravenous infusion at a dosage of about 10 mg/m² to about 60 mg/m².
56. The method of embodiment Error! Reference source not found., wherein the alvocidib is administered at an overall dosage of about 20 mg/m² to about 100 mg/m².
57. The method of embodiment Error! Reference source not found., wherein the alvocidib is administered as an intravenous infusion.
58. The method of embodiment Error! Reference source not found., wherein the intravenous infusion is over about 1 hour.
59. The method of claim Error! Reference source not found., wherein the alvocidib is administered at a dosage of about 90 mg/m².
60. The method of any of embodiments Error! Reference source not found.-Error!Reference source not found., wherein the decitabine is administered at a daily dosage of about 10 mg/m² to about 30 mg/m².
61. The method of embodiment Error! Reference source not found., wherein the decitabine is administered at a daily dosage of about 20 mg/m².
62. The method of any of embodiments 1-Error! Reference source not found., wherein the patient is further administered a tumor lysis syndrome prophylaxis.
63. The method of embodiment Error! Reference source not found., wherein the tumor lysis syndrome prophylaxis comprises intravenous hydration with a 0.45% aqueous NaCl.
64. The method of embodiment Error! Reference source not found., wherein the tumor lysis syndrome prophylaxis comprises administering one or more of allopurinol, an oral phosphate binder, replacement of fluid losses, and an anti-diarrheal medication.
65. The method of embodiment Error! Reference source not found., wherein the tumor lysis syndrome prophylaxis is administered prior to first HMA dose.
66. The method of embodiment Error! Reference source not found., wherein the tumor lysis syndrome prophylaxis is administered prior to first alvocidib dose.
67. The method of any of embodiments 1-Error! Reference source not found., wherein the patient is 18 years old or greater.
68. The method of any of embodiments 1-Error! Reference source not found., wherein the patient has an Eastern Cooperative Oncology Group (ECOG) Performance Status (PS) score which is less than or equal to 2.
69. The method of any of embodiments 1-9, wherein the patient has a life expectancy of greater than or equal to 3 months.
70. The method of any of embodiments 1-Error! Reference source not found., wherein the patient meets the following criteria based on laboratory data:
a) wherein the patient's serum creatinine is less than or equal to 1.8 times the upper limit of the normal (ULN) range;
b) wherein the patient's total bilirubin is less than or equal to 2 times the ULN range, and
c) wherein the patient's aspartate transaminase (AST) and alanine transaminase (ALT) are less than or equal to 3 times the ULN range.
71. The method of any of embodiments 1-Error! Reference source not found., wherein the patient does not have a concomitant severe cardiovascular disease.
72. The method of any of embodiments 1-Error! Reference source not found., wherein the patient does not have a condition selected from New York Heart Association (NYHA) Functional Class III or IV heart disease, National Cancer Institute (NCI) Common Terminology Criteria for Adverse Events (CTCAE) v5.0 grade equal to or greater than 3 arrhythmia, angina pectoris, abnormal electrocardiogram findings, interstitial pneumonia, and pulmonary fibrosis.
73. The method of any of embodiments 1-Error! Reference source not found., wherein the patient has not had myocardial infarction within 6 months before the treatment.
74. The method of any of embodiments 1-Error! Reference source not found., wherein the patient does not have a concomitant malignancy requiring chemotherapy, or a concomitant malignancy for which the patient received chemotherapy within 6 months prior to treatment, with the proviso that the malignancy is not selected from basal and squamous cell carcinoma of the skin.
75. The method of any of embodiments 1-Error! Reference source not found., wherein the patient does not have an uncontrolled or uncontrollable infection, or a Grade equal to or greater than 3 infection according to NCI CTCAE v5.0. 76. The method of any of embodiments 1-Error! Reference source not found., wherein the patient does not have a dry tap on bone marrow aspiration.
77. The method of any of embodiments 1-Error! Reference source not found., wherein the patient does not have a concurrent autoimmune disease or a history of chronic or recurrent autoimmune disease.
78. The method of any of embodiments 1-Error! Reference source not found., wherein the patient does not require a long-term systemic steroid therapy greater than the equivalent of 20 mg of prednisone daily.
79. The method of any of embodiments 1-Error! Reference source not found., wherein the patient does not have another documented malignancy within the past year.
80. The method of any of embodiments 1-Error! Reference source not found., wherein the patient does not have Grade equal to or greater than 2 hemorrhage according to NCI CTCAE v5.0. 81. The method of any of embodiments 1-Error! Reference source not found., wherein the patient is not pregnant or breastfeeding.
82. The method of any of embodiments 1-Error! Reference source not found., wherein the patient has not previously received alvocidib or another cyclin-dependent kinase 9 (CDK9) inhibitor.
83. The method of any of embodiments 1-Error! Reference source not found., further comprising determining a BH3 profile for the patient's tumor cell specimen.
84. The method of embodiment Error! Reference source not found., further comprising classifying the patient for likelihood of response to MDS treatment based on the patient's BH3 profile.
85. The method of any of embodiment Error! Reference source not found. or Error!Reference source not found., further comprising measurement of an additional biomarker associated with MDS.
86. The method of embodiment 68, wherein the additional biomarker associated with MDS is selected from the group consisting of nucleic acids, proteins, lipids, and metabolites.
87. The method of embodiment 68, wherein the additional biomarker associated with MDS is MCL-1. 88. The method of any of embodiments Error! Reference source not found.-70, wherein the BH3 profile is determined by flow cytometry.
89. A method for determining a response to MDS treatment comprising administering a hypomethylating agent and alvocidib to a patient with previously untreated MDS, the method comprising determining a BH3 profile for the patient's tumor cell specimen, and classifying the patient for likelihood of response to MDS treatment.
90. The method of embodiment Error! Reference source not found., further comprising measurement of an additional biomarker associated with MDS.
91. The method of embodiment Error! Reference source not found., wherein the additional biomarker is selected from the group consisting of nucleic acids, proteins, lipids, and metabolites.
92. The method of embodiment Error! Reference source not found., wherein the additional biomarker is MCL-1. 93. The method of any of embodiments Error! Reference source not found.-Error!Reference source not found., wherein the BH3 profile is determined by flow cytometry.
94. A method of treating a patient with myelodysplastic syndrome (MDS) comprising administering to the patient a therapeutically effective amount of a hypomethylating agent (HMA) selected from azacitidine and decitabine and subsequently administering to the patient a therapeutically effective amount of alvocidib.
95. The method of embodiment Error! Reference source not found. wherein the HMA is administered intravenously.
96. The method of embodiment Error! Reference source not found. wherein the HMA is decitabine administered at a dose of about 10 mg/m² to about 30 mg/m² for from about 1 to about 3 hours, once to three times/day.
97. The method of embodiment Error! Reference source not found. wherein the decitabine is administered once/day for 3 to 7 days.
98. The method of embodiment Error! Reference source not found. where the decitabine is administered once/day for 5 days.
99. The method of embodiment Error! Reference source not found. wherein the decitabine is administered at a dose of about 20 mg/m² in a one hour infusion.
100. The method of embodiment Error! Reference source not found. wherein the alvocidib is administered at a rate of about 10 mg/m² to about 120 mg/m². 101. The method of embodiment Error! Reference source not found. wherein the alvocidib is administered two days after the cessation of the decitabine administration.
102. The method of embodiment Error! Reference source not found. wherein the alvocidib is administered two days after the cessation of the decitabine administration.
103. The method of embodiment Error! Reference source not found. wherein a portion of the alvocidib is administered as a bolus dose of from about 10 mg/m² to about 50 mg/m² over a period of about 10 minutes to about 60 minutes.
104. The method of embodiment Error! Reference source not found., wherein the bolus dose is administered over a period of about 30 minutes.
105. The method of embodiment Error! Reference source not found. wherein the bolus dose is from about 20 mg/m² to about 30 mg/m².
106. The method of embodiment Error! Reference source not found. wherein from about 10 mg/m² to about 60 mg/m² of alvocidib is administered intravenously over a period of about 2 hours to about 6 hours.
107. The method of embodiment Error! Reference source not found., wherein the alvocidib is administered over a period of about 4 hour.
108. The method of embodiment Error! Reference source not found. wherein the dose of the alvocidib is from about 20 mg/m² to about 60 mg/m².
109. The method of embodiment Error! Reference source not found. wherein the alvocidib is administered intravenously at a dose of about 90 mg/m² over a period of about 20 minutes to about 120 minutes.
110. The method of embodiment Error! Reference source not found., wherein the alvocidib is administered over a period of about 1 hour.
111. The method of embodiment Error! Reference source not found. wherein the administration of the alvocidib by intravenous infusion is begun within about 30 minutes of the completion of the bolus dose.
112. The method of embodiment Error! Reference source not found. wherein the HMA is azacitidine at a dose of about 30 to about 90 mg/m².
113. The method of embodiment Error! Reference source not found. wherein the dose is about 75 mg/m² per day.
114. The method of embodiment Error! Reference source not found. wherein the azacitidine is administered for seven days as an intravenous bolus injection over about 10 to about 40 minutes or subcutaneous injection.
115. The method of embodiment Error! Reference source not found. wherein the alvocidib is administered intravenously two days after the cessation of azacitidine administration.
116. The method of embodiment Error! Reference source not found. wherein the alvocidib is administered on day 10 after the commencement of azacitidine administration with no azacitidine administration on days 8 and 9.
117. The method of embodiment Error! Reference source not found. wherein 90 mg/m² of the alvocidib is administered intravenously over a period of about 20 minutes to about 120 minutes.
118. The method of embodiment Error! Reference source not found., wherein the alvocidib is administered over a period of about 1 hour.
119. The method of embodiment Error! Reference source not found. wherein the azacitidine is administered at a dose of about 30 to about 90 mg/m²/day for five consecutive days, followed by azacitidine-free days 6 and 7, further followed by intravenous administration of azacitidine at a dose of about 30 to about 90 mg/m² on days 8 and 9, and further followed by intravenous administration of the alvocidib on day 10.
120. The method of embodiment Error! Reference source not found. wherein the azacitidine is administered at a dose of about 75 mg/m²/day by intravenous bolus injection on days 1 to 5 and days 8 and 9, and wherein the alvocidib is administered at a dose of about 90 mg/m² over a period of about one hour by intravenous infusion on day 10.
121. The method of embodiment Error! Reference source not found. wherein the treatment is repeated at least once.
122. The method of embodiment Error! Reference source not found. wherein the treatment is repeated at least once.
123. The method of embodiment Error! Reference source not found. wherein a treatment cycle comprises 28 days.
124. The method of embodiment Error! Reference source not found. wherein the treatment cycle is repeated at least once.
125. The method of embodiment Error! Reference source not found. wherein the treatment is repeated for at least 4 cycles.
126. The method of embodiment Error! Reference source not found. wherein a treatment cycle comprises four to six weeks.
127. The method of embodiment Error! Reference source not found. wherein the treatment is repeated for at least 4 cycles.
128. The method of embodiment Error! Reference source not found. wherein the HMA is administered orally.
129. The method of embodiment Error! Reference source not found. wherein the HMA is administered as a prodrug.
130. The method of embodiment Error! Reference source not found. wherein the HMA is administered in combination with a cytidine deaminase inhibitor.
131. The method of embodiment Error! Reference source not found. wherein the HMA is decitabine.
132. The method of embodiment Error! Reference source not found. wherein the cytidine deaminase inhibitor is cedazuridine.
133. The method of embodiment Error! Reference source not found. wherein the HMA is an azacitidine phosphate prodrug.
134. The method of embodiment Error! Reference source not found. wherein the azacitidine prodrug has the formula

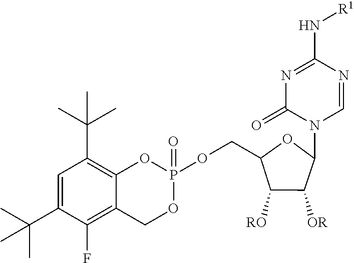

where R and R1 are independently H or CO2 (C1-C6 alkyl).
135. The method of embodiment Error! Reference source not found. wherein the HMA is the composition cc-486.
136. The method of embodiment Error! Reference source not found. wherein the HMA is azacitidine administered as 2′,3′,5′-triacetyl-5-azacitidine.
137. The method of any of the preceding embodiments, wherein the patient has one or more mutations in one or more of RUNX1, SRSF2, SF3BI, U2AF1, ZRSR2, ASXL1, EZH2, BCOR and STAG2. 138. The method of embodiment 137, wherein the patient has one or more mutations in RUNX1.
139. The method of embodiment 137 or 138, wherein the patient has one or more mutations in ASXL1.
140. The method of any one of embodiments 137-139, wherein the patient has one or more mutations in one, two, three, four or five of RUNX1, SRSF2, SF3BI, U2AF1, ZRSR2, ASXL1, EZH2, BCOR and STAG2.
141. The method of any one of the preceding embodiments, wherein the MDS is MCL-1 dependent.

### EXAMPLES

The following examples illustrate specific aspects of the instant description. The examples should not be construed as limiting, as the examples merely provide specific understanding and practice of the embodiments and their various aspects.

### Abbreviations

AE Adverse event
ACM Cytarabine and mitoxantrone
ALL Acute lymphocytic leukemia
ALP Alkaline phosphatase
ALT Alanine aminotransferase
AML Acute myeloid leukemia
aPTT Activated partial thromboplastin time
AST Aspartate aminotransferase
AUC Area under the concentration time curve
AUC₀₋₂₄ AUC from 0 to 24 hours
AUC_{0-inf} AUC from 0 to infinity
AUCₜ AUC from 0 to time t
AZA Azacitidine
B-CLL B-cell chronic lymphocytic leukemia
p-HCG Beta human chorionic gonadotropin
AUC Area under the curve
BM Bone marrow
BSA Body surface area
BUN Blood urea nitrogen
CBC Complete blood count
CDK9 Cyclin dependent kinase 9
CFR Code of Federal Regulations
CI Confidence interval
CIV Continuous intravenous
CL Clearance using noncompartmental methods
Cₘₐₓ Maximum concentration
CR Complete response
CRA Clinical research associate
CRF Case report form
CRi Complete Response with incomplete blood count recovery
CRO Contract Research Organizaiton
CRR Complete Response Rate (CR/CRi/CRmarrow/PR/HI)
CSR Clinical Study Report
CTCAE Common Terminology Criteria for Adverse Events
DCF Data clarification form
DEC Decitabine
DLT Dose-limiting toxicity
DSMB Data Safety Monitoring Board
ECOG Eastern Cooperative Oncology Group
ECG Electrocardiogram
ECHO Echocardiogram
eCRF Electronic case report form
ESA Erythropoietin-stimulating agent
FAB French-American-British (classification system)
FDA Food and Drug Administration
GCP Good Clinical Practice
hERG Human Ether-a-go-go-Related Gene
Hgb Hemoglobin
HI Hematologic Improvement
HMA Hypomethylating agent
IC₅₀ Inhibitory concentration in 50% of test subjects
IEC Independent Ethics Committee
IHC Immunohistochemistry
IND Investigational New Drug (application)
IPSS-R Revised International Prognostic Scoring System
IRB Institutional Review Board
ITT Intent-to-treat
IV Intravenous(ly)
IVI Intravenous infusion
IWG International Working Group
LDH Lactate dehydrogenase
LVEF Left ventricular ejection fraction
MCL-1 Myeloid cell leukemia 1
MDS Myelodysplastic syndrome
MedDRA Medical Dictionary for Regulatory Activities
MTD Maximum tolerated dose
MUGA Multigated acquisition (scan)
NCI National Cancer Institute
NYHA New York Heart Association
OS Overall survival
PD Progressive disease
PD Pharmacodynamic
PK Pharmacokinetic
PR Partial response
PRN As needed
PS Performance status
PT Prothrombin time
QTc QT interval (corrected)
RBC Red blood cell
RP2D Recommended Phase 2 Dose
RT-aPCR Quantitative reverse transcription-polymerase chain reaction
SAE Serious adverse event
SAS Statistical Analysis System (software)
SD Stable disease
SGOT (AST) Serum glutamic-oxaloacetic transaminase
SGPT (ALT) Serum glutamic-pyruvic transaminase
SIV Study initiation visit
SOP Standard operating procedure(s)
SPRT Sequential Probability Ratio Test
T_{1/2} Half-life
TEAE Treatement-emergent adverse event
TLS Tumor lysis syndrome
Tₘₐₓ Time to maximum concentration
ULN Upper limit of normal
WHO World Health Organization
XIAP X-linked inhibitor of apoptosis

### Example 1: Alvocidib Shows Clinical Activity in Secondary AML

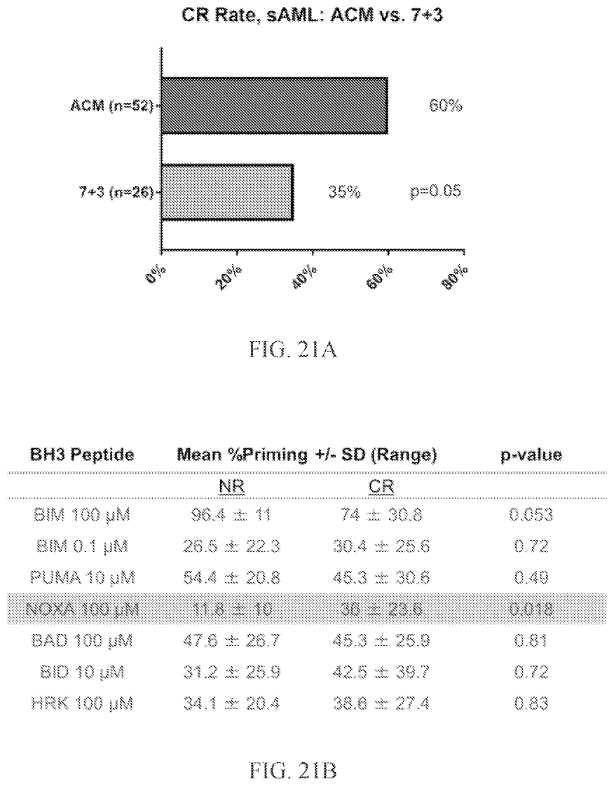

Alvocidib shows clinical activity in secondary AML (FIG. 21). An ACM (alvocidib+cytarabine+mitoxantrone) regimen demonstrated a significant improvement in CR rates in clinical studies of secondary AML patients. The ACM regimen outperformed 7+3 chemotherapy. CR/CRi rate for treatment groups having secondary AML was assessed (FIG. 14A). AML patients demonstrated a significant improvement in CR rates over 7+3 chemotherapy patients. Mitochondrial profiling performed on pre-treatment bone marrow samples (n=24) and correlated with response. Mann-Whitney tests were used for each mimetic peptide. Patient responses to ACM treatment correlated with NOXA priming in AML patients (FIG. 14B).

### Example 2: Alvocidib Reduces RNA Pol II Phosphorylation and MCL-1 Expression In Vitro

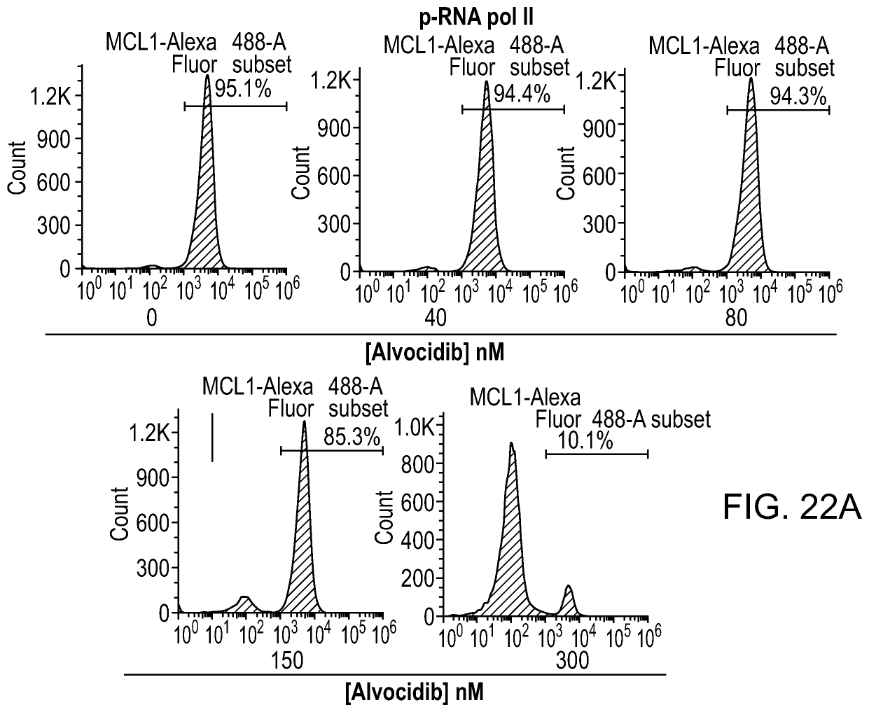

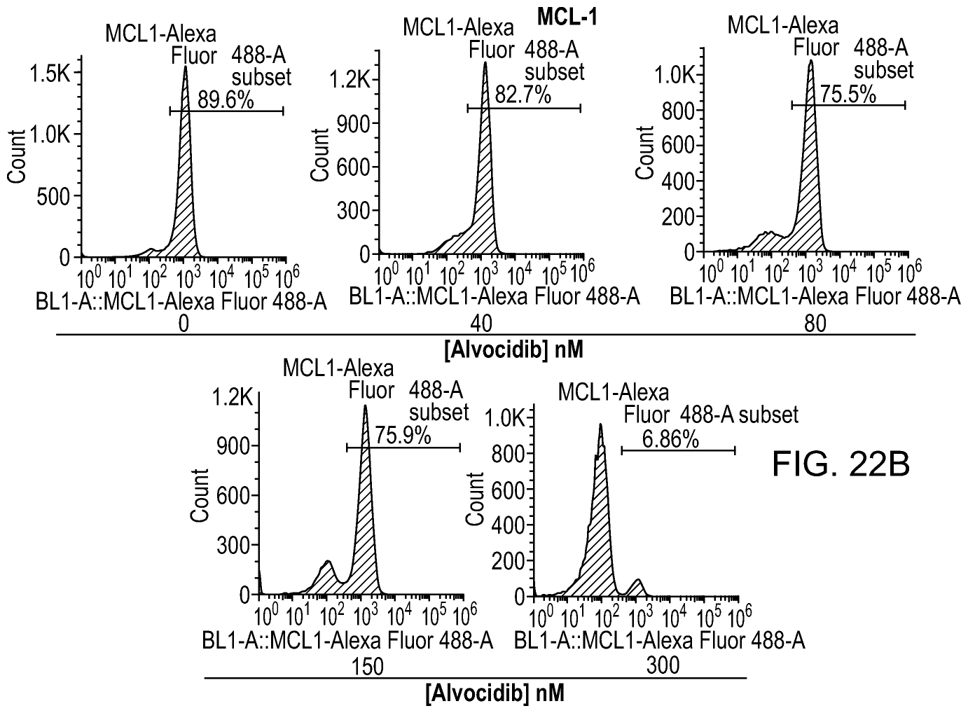

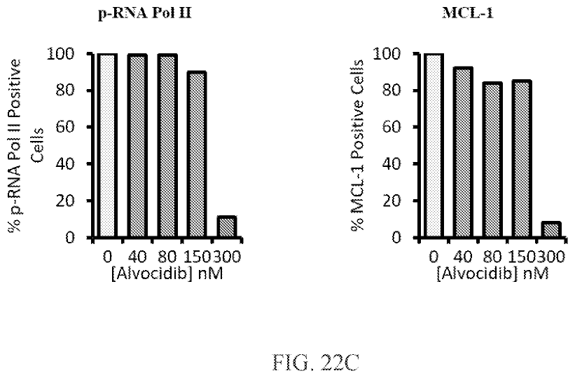

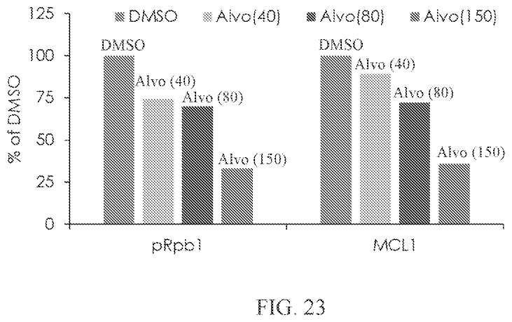

It has been shown that alvocidib reduces RNA pol II phosphorylation and MCL-1 expression in vitro. FIGS. 22A and 22B show that alvocidib inhibits RNA pol II phosphorylation (a primary CDK9 substrate) and MCL-1 expression (a PD marker encoded by a gene regulated by phosphorylation of RNA pol II by CDK9) in a dose-dependent fashion in MV-4-11 AML cells, as measured by flow cytometry following 24-hour treatment. FIG. 22C is a graphical representation of the flow cytometry data shown in FIGS. 22A and 22B. Phospho-RNA polymerase (pRpb1) and MCL-1 (MCL1) levels are decreased after alvocidib treatment in MDS patient cells (FIG. 23). Levels are indicated as a percent relative to levels measured for a DMSO treated sample. Numbers in parenthesis represent concentrations in nM for each alvocidib (Alvo) treatment.

### Example 3: HMAs Increase NOXA Expression In Vitro

An experiment evaluating whether the hypomethylating agents (HMAs) decitabine and azacitidine approved for treatment of MDS can influence MCL-1 dependency through expression of pro-apoptotic proteins like NOXA has been conducted.

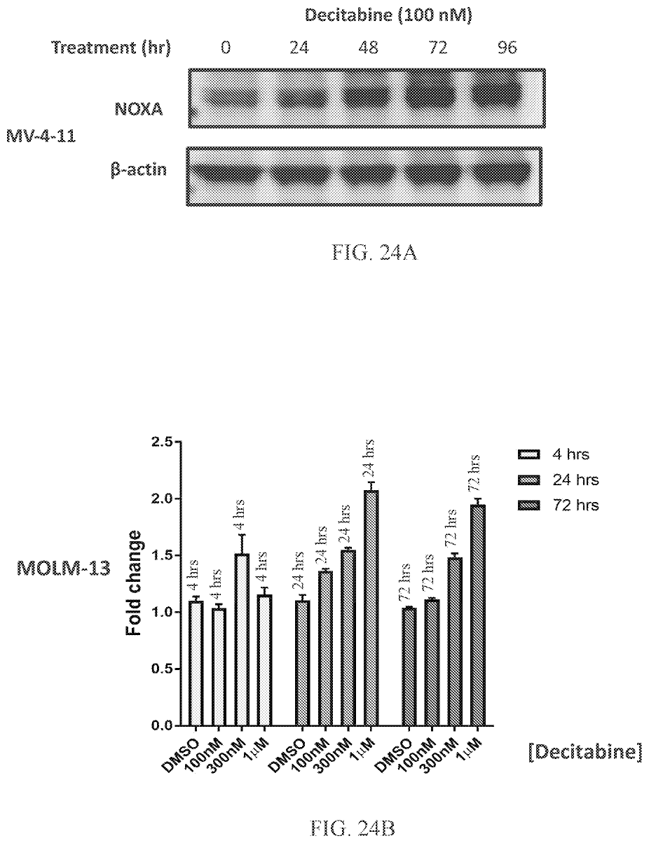

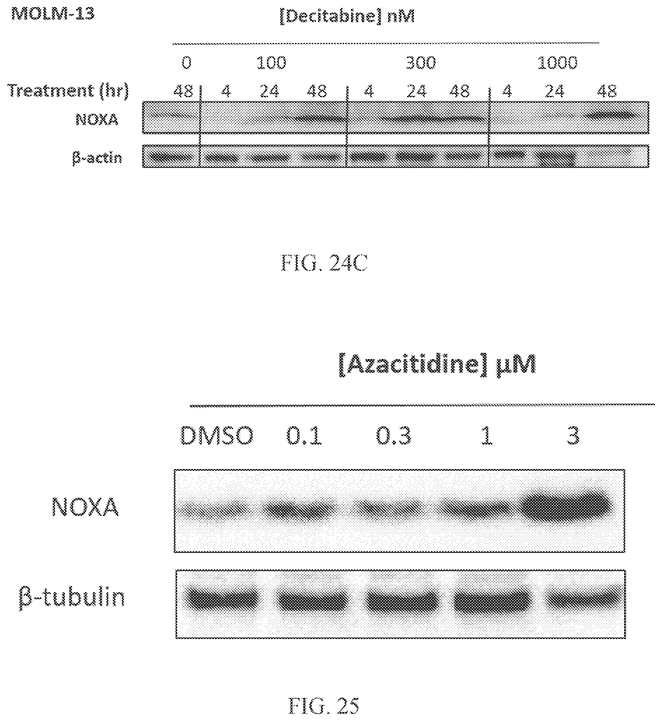

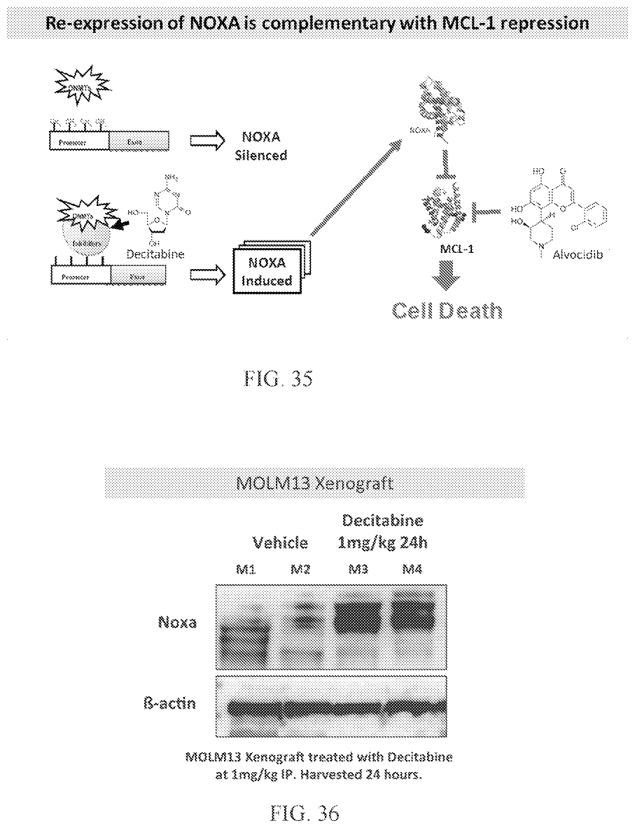

HMAs (e.g., decitabine and azacitidine) increase NOXA expression in vitro (FIGS. 24A-24C and FIGS. 35-36 (decitabine) and FIG. 25 (azacitidine)). In the decitabine experiments (FIGS. 24 and 36), the MV-4-11 AML cell line and MOLM13 cell line model for secondary AML (prior MDS) were used. MV-4-11 AML cells (or MOLM13 cells as indicated) were treated with indicated concentrations of decitabine for indicated times, and protein was assessed by Western blot for NOXA expression. A time-dependent increase of NOXA expression was observed. FIGS. 24A and 24C are Western blots and FIG. 24B is a bar graph of mRNA concentrations. Together, FIGS. 24A-C demonstrate that HMAs, such as decitabine, induce NOXA re-expression. FIG. 36 is a Western blot further demonstrating that decitabine effects an increase in NOXA expression. The Western blot was prepared from a MOLM13 xenograft treated with decitabine at 1 mg/kg IP for 24 hours. FIG. 35 illustrates decitabine-mediated re-expression of NOXA is complementary with MCL-1 repression by alvocidib.

In the azacitidine experiments (FIG. 25), MV-4-11 AML cells were treated with indicated concentrations of azacitidine for 24 hours and protein assessed by Western blot for NOXA expression. A dose-dependent increase of NOXA expression was observed.

Additionally, an assay has been tested in bone marrow mononuclear cells (BMMCs) from AML and MDS patients to examine MCL-1 dependency prior to therapy with alvocidib. Approximately 25% of AML patient BMMCs and 60% of MDS patient BMMCs are MCL-1 dependent. Azacitidine and decitabine increased NOXA expression in AML cell line models. Additionally, azacitidine treatment sensitized MV4-11 cells to MCL-1 dependent apoptosis. Alvocidib treatment resulted in a dose dependent reduction in pRpb1 and MCL1 in primary MDS BMMCs. Treatment with alvocidib and azacytidine resulted in IC50 values for ˜100 nM and ˜1000 nM in MDS BMMC's respectively. Combination studies are currently under investigation to determine whether enhanced anti-cancer activity is observed through pharmacologic downregulation of MCL-1 via CDK9 inhibition by alvocidib and upregulation of the MCL-1 antagonist NOXA after HMA exposure.

### Example 4: Synergistic Effect of HMAs in Combination with Alvocidib

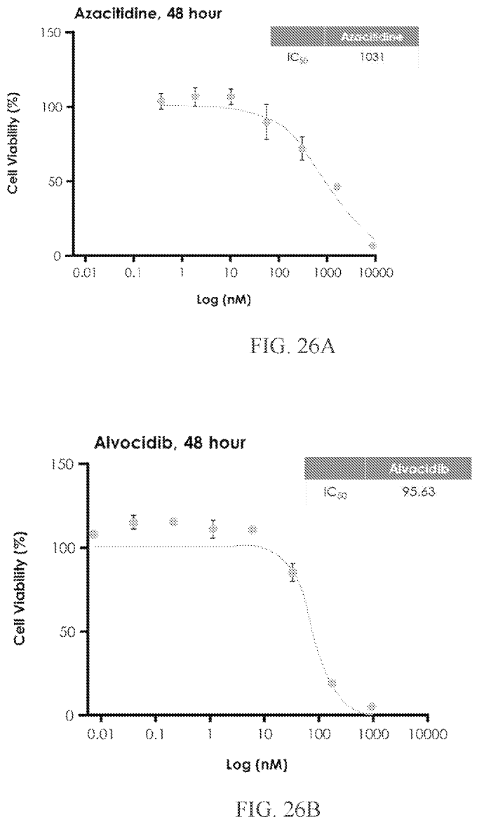

FIGS. 26A and 26B are scatter plots showing cell viability following 48 hours treatment at indicated concentrations of azacytidine (alone) or alvocidib (alone). FIG. 26A is a plot of cell viability following 48-hour treatment against indicated concentrations of azacitidine. IC₅₀ for azacitidine treatment was 1031 nM. FIG. 26B is a plot of cell viability following 48-hour treatment against indicated concentrations of alvocidib. IC₅₀ for azacitidine treatment was 95.63 nM.

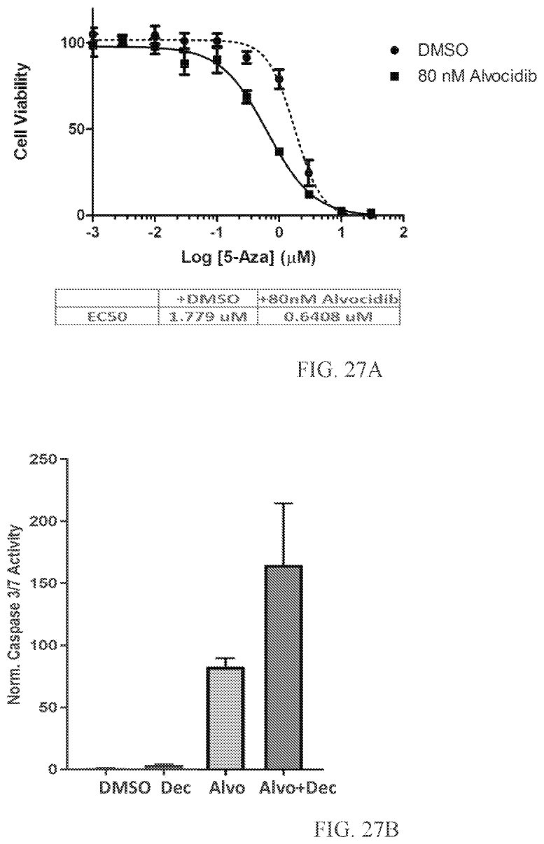

A synergy between alvocidib and HMAs to induce apoptosis is demonstrated in FIG. 27. FIG. 27A is a plot of cell viability in cells treated with DMSO (control) and indicated concentrations of azacytidine or 80 nM alvocibib and indicated concentrations of azacitidine. The lower panel of FIG. 27A lists EC50 values determined from the plot of cell viability. Cell viability was assessed (Celltiter-glo) in MV4-11 cells. Cells were treated for 24 hours with 80 nM alvocidib at various concentrations of azacitidine. Additive anti-proliferative activity was observed in combination treatment. FIG. 27B is a bar graph of caspase activity in cells treated with DMSO (control), decitabine, alvocidib, or decitabine and alvocidib. Cells were treated for 24 hours. Caspase activity was measured using Caspase-glo. Decitabine and alvocidib in combination demonstrated synergistic apoptosis activity.

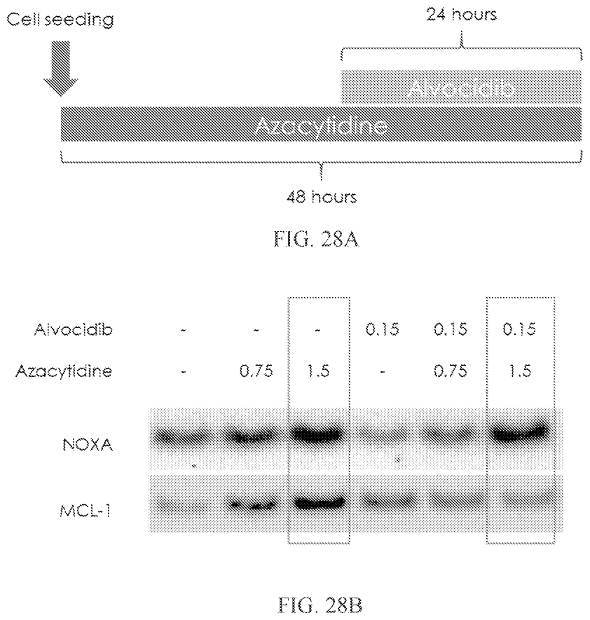

Alvocidib inhibits upregulation of MCL-1 by azacytidine without affecting NOXA induction in MV-4-11 cells (FIG. 28). FIG. 28A is an overview of an experimental used to evaluate an interaction between alvocidib and azacytidine with regard to MCL-1 and NOXA expression. FIG. 28B is a Western blot showing NOXA and MCL-1 levels in cells treated according to the experiment summarized in FIG. 28A at indicated drug concentrations.

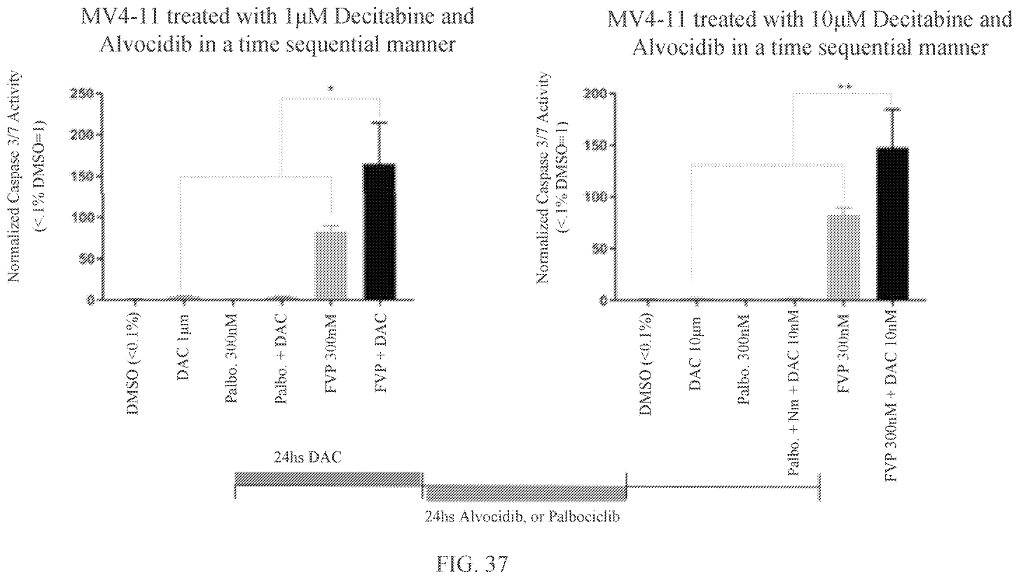

FIG. 37 demonstrates that administration of decitabine followed by administration of alvocidib results in a synergistic increase in normalized caspase 3/7 activity in an AML cell line. The lower panel of FIG. 37 illustrates a dosing regimen used to assess any synergy between decitabine and alvocidib. Cells were exposed to decitabine (DAC) for 24 hours followed by 24 hours exposure to ALV or Palbo. The upper left panel of FIG. 37 and the upper right panel of FIG. 37 are bar graphs demonstrating a synergy between DAC and AML administered at indicated concentrations according to the protocol shown in the lower panel of FIG. 37. In all experiments Palbo was evaluated at 300 nM and ALV was evaluated at 300 nM. Without wishing to be bound by theory, it is hypothesized that the synergistic effect of decitabine followed by alvocidib effects through a CDK9-dependent mechanism.

### Example 5: HMAs Increase MCL-1 Dependency

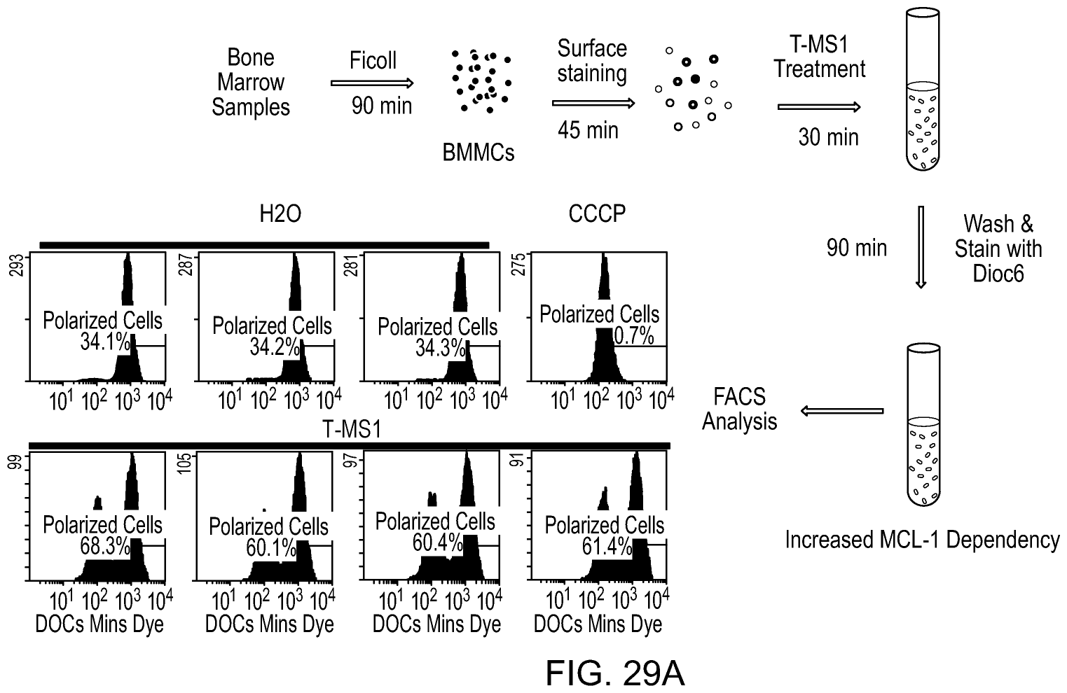

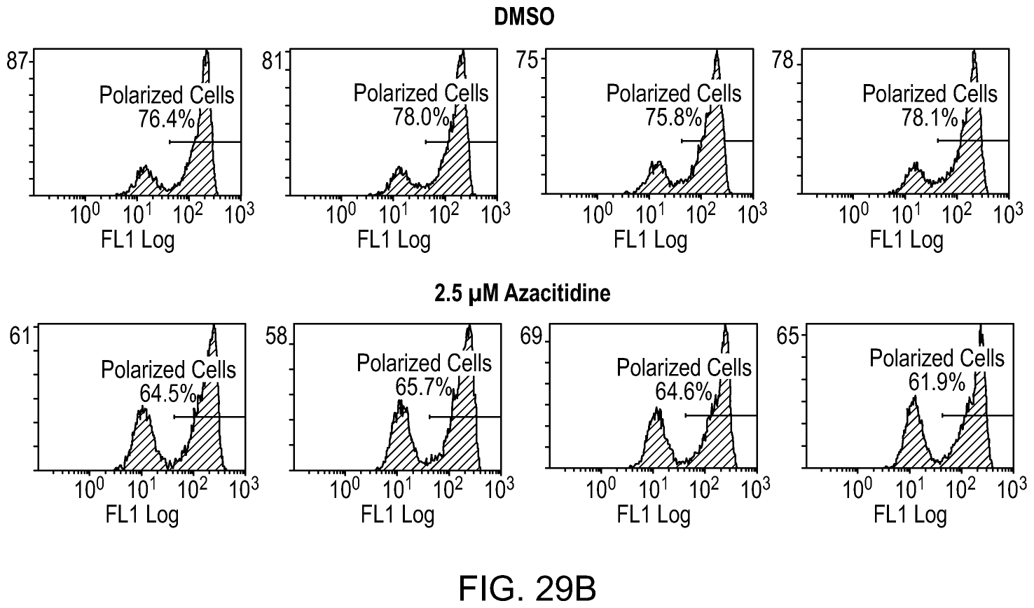

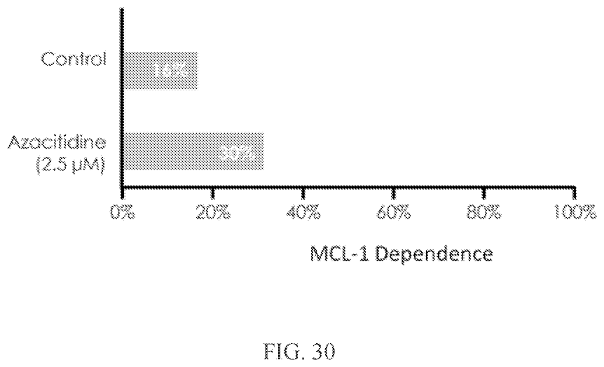

FIG. 37 show the results of an MCL-1 dependency assay that demonstrate that HMA treatment increases MCL-1 dependency. BMMC are isolated by Ficoll gradient, stained with cell surface markers and incubated with water or T-MS1 (MCL-1 specific peptide) and stained with Dioc6 to determine mitochondrial membrane potential. Loss of fluorescence indicates increased MCL-1 dependency. FIG. 29B shows flow cytometry data demonstrating that MV-4-11 cells treated with azacytidine for 24 hours showed increased MCL-1 dependency compared with DMSO controls. The lower panel of FIG. 29B lists % priming under each treatment condition.

FIG. 29B is a bar graph demonstrating that HMA treatment increases sensitivity to MCL-1 suppression. MV-4-11 cells treated with azacitidine demonstrated increased sensitivity to MCL-1 suppression. Control (DMSO) showed 16.1% MCL-1 dependence and azacitidine at 2.5 μM showed 29.6% MCL-1 dependence.

### Example 6: Sequential Dosing of Alvocidib and HMAs In Vitro

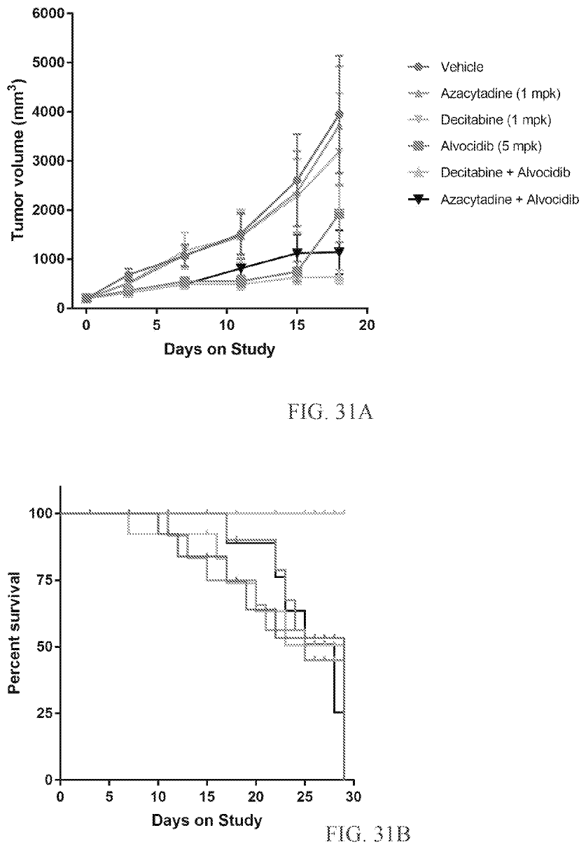

FIGS. 31A and 31B show results from sequential dosing of alvocidib and HMAs in a MOLM13 model for MDS/sAML. HMA dosing sensitized AML cells to sequential dosing and improved survival in vivo. Alvocidib, azacytidine, and decitabine activity were assessed in the MOLM13 xenograft model. FIG. 31A is a plot of tumor volume over time and FIG. 31B is a plot of percent survival over time. Mice were dosed with alvocidib, qdx2, and/or an HMA, qdx3 on a weekly basis. Doses are indicated in mg/kg (mpk).

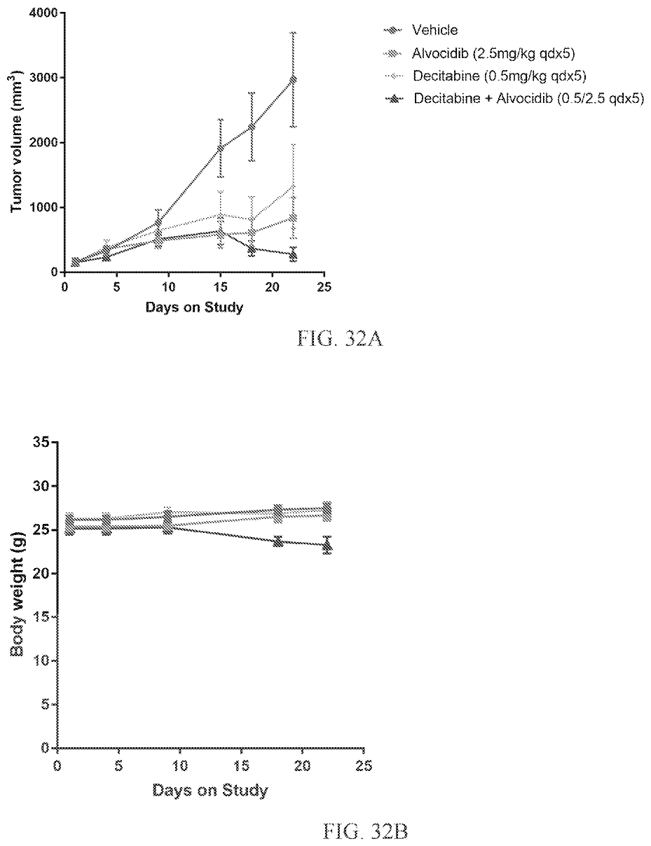

FIG. 29B show results from aggressive daily dosing of alvocidib and decitabine in the MOLM13 model. Tumor bearing mice were treated. Doses and schedule are indicated. FIG. 32A plots tumor volume following treatment and FIG. 32B plots body weight following treatment. As a single agent, alvocidib achieved tumor growth inhibition (% TGI) of 75.8. Decitabine achieved a % TGI of 58.6 as a single agent. In combination, decitabine and alvocidib achieved a % TGI of 95.8.

### Example 7: Efficacy of Alvocidib Alone or in Combination with HMA in AML Xenograft Models

The efficacy of alvocidib alone or the combination with HMA has been explored in several AML xenograft models to support studies in high-risk MDS patients. Treatment with alvocidib alone resulted in complete abrogation of total flux count in luciferase expressing MV4-11 model and 30.8% TGI in a THP-1 AML model. The combination of azacitidine or decitabine with alvocidib was active in the OCI AML3 xenograft model, yielding up to 62.6 or 78.2% tumor growth inhibition (% TGI), respectively. While alvocidib as single agent has shown 55.0% TGI. Survival data also supports the combination of alvocidib and HMA.

These in vitro studies and pre-clinical data suggest that an alvocidib/HMA combination may constitute a viable therapeutic regimen whose rationale focuses on hypertargeting of NOXA/MCL-1. Taken together, the in vitro and in vivo studies indicate that combination of alvocidib and HMA reagents can drive the AML/MDS cells toward MCL-1 dependent apoptosis.

### Example 8: Combination Treatment Regimen Effects

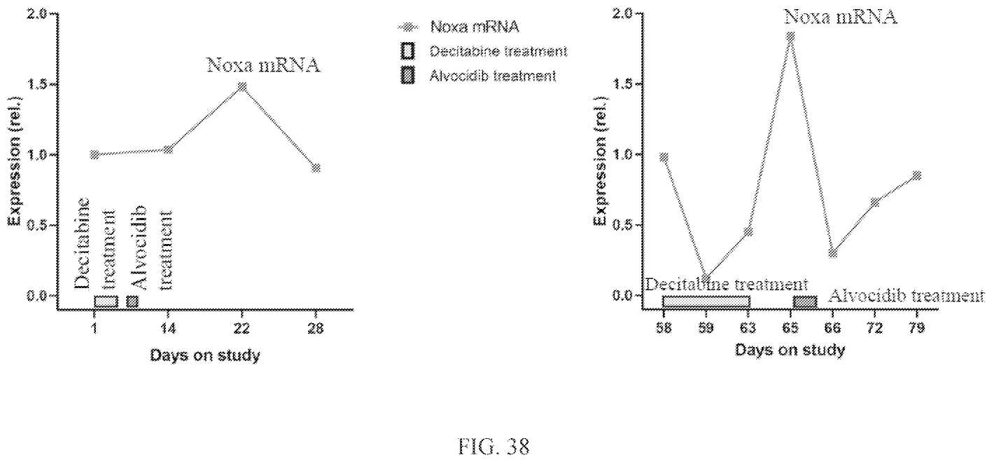

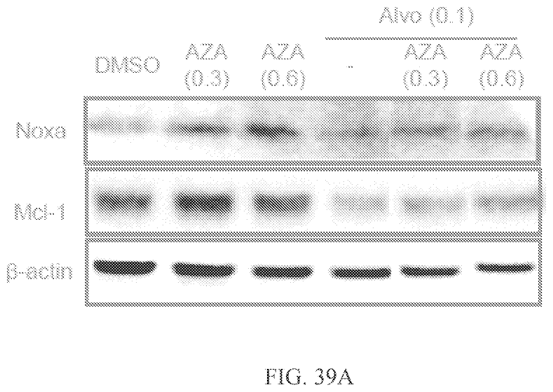

To assess the effects of hypomethylating agent (HMA) treatment on the expression of NOXA in the combination treatment regimen, the expression of NOXA mRNA was measured using standard quantitative real-time PCR (qPCR) technique in PBMCs harvested from patients at the times indicated, as shown in FIG. 38. For reference, treatment timing and duration is also indicated (colored bars).

FIG. 38 depicts scatter plots showing average trends of NOXA mRNA levels in Cohort 1 patients of a TPI-ALV-102 phase 1b/2, open-label clinical study (https://www.ClinicalTrials.gov identifier NCT03593915) to determine preliminary safety of alvocidib when administered in sequence after decitabine (“in sequence” treatment) in patients with MDS. The left and right panels of FIG. 38 demonstrate that NOXA expression peaks several days (about 2 weeks shown in the left panel and about two days in the left panel) after termination of HMA administration. The colored bars in the scatter plots illustrate the timing and duration of drug administration.

Increased NOXA expression following HMA treatment was observed, both in cycle 1 of treatment (FIG. 38, left panel), and in subsequent cycles (showing cycle 3, FIG. 38, right panel). In cycle 1 treatment, a peak of NOXA expression was observed at day 22. Following these observations, additional collections were added to the protocol and laboratory manual allowing for better resolution during the treatment. Cycle 3 treatment measurements of NOXA are shown at FIG. 38, right panel. Immediately following treatment initiation, a loss in NOXA is seen, which may be a result of blast cell death, which ultimately leads to an increase observed by day 65. Alvocidib was dosed here at a local peak of NOXA expression.

These results indicate that optimal synergy of the combination of HMA (as exemplified by decitabine) and alvocidib may be achieved by administering alvocidib during a period of maximum or elevated NOXA expression achieved following decitabine administration.

### Example 9: Patient Populations, Inclusion and Exclusion Criteria

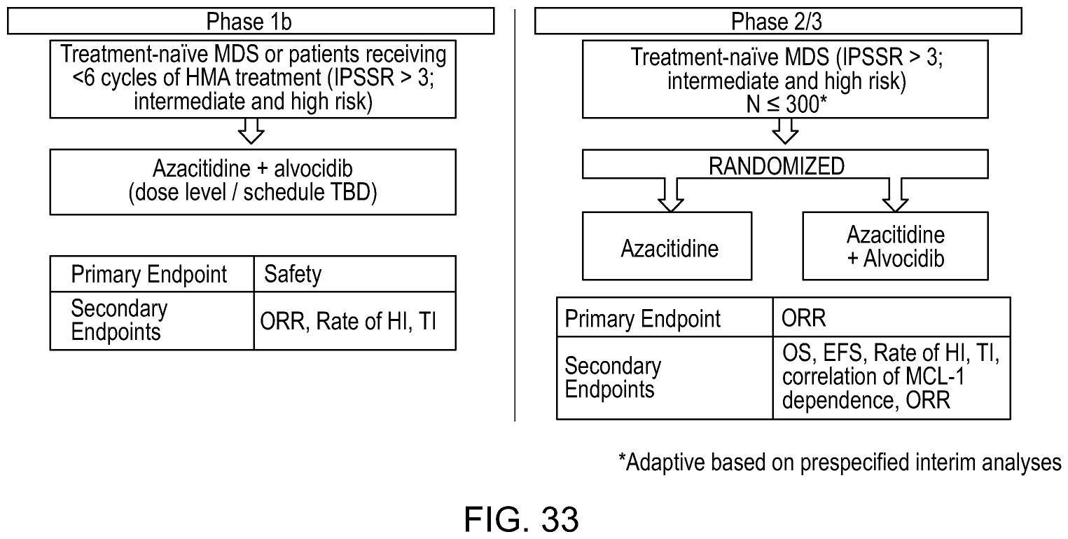

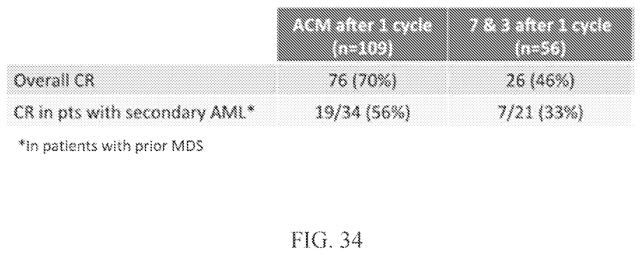

FIG. 33 is an illustration of a study schema for a clinical trial including an azacitidine arm. The clinical trial will evaluate HMAs+/−alvocidib in newly diagnosed intermediate and high-risk myelodysplastic syndromes.

A Phase 1b/2 clinical study of alvocidib administered in sequence after an HMA (e.g., azacytidine or decitabine) in patients with intermediate to high risk MDS is being conducted (Zella 102). Enrollment includes MDS patients (Phase 1b) with previously untreated MDS and patients who received fewer than six (6) cycles of previous HMAs, as well as (Phase 2) untreated patients with de novo (cause unknown) or secondary MDS (treatment-related). This includes all French-American-British (FAB) subtypes (refractory anemia, refractory anemia with ringed sideroblasts, refractory anemia with excess blasts, refractory anemia with excess blasts in transformation, and chronic myelomonocytic leukemia), and intermediate and above per the Revised International Prognostic Scoring System (IPSS-R) groups. The primary objective is to determine the maximum tolerated dose and recommended Phase 2 dose of alvocidib when administered in sequence with decitabine or azacitidine. Key Phase 2 endpoints will include complete response rate and reduced transfusion dependency.

### Patient Populations:

Patients with previously untreated MDS
Patients with MDS who have received fewer than 6 cycles of treatment with hypomethylating agents (HMAs)
Patients with de novo (cause unknown) or secondary MDS (treatment-related) who are not eligible for intensive induction chemotherapy or stem cell transplant
All French-American-British (FAB) subtypes (refractory anemia, refractory anemia with ringed sideroblasts, refractory anemia with excess blasts, refractory anemia with excess blasts in transformation, and chronic myelomonocytic leukemia)
Intermediate and above per the Revised International Prognostic Scoring System (IPSS-R) groups

### Inclusion Criteria:

To be eligible for participation in the study, patients must meet all of the following inclusion criteria:
1. Aged >18 years
2. Phase 1b: Patients with previously untreated MDS and patients with MDS who received fewer than six (6) cycles of previous HMAs
Phase 2: Untreated patients with de novo or secondary MDS
3. Patients with an Eastern Cooperative Oncology Group (ECOG) Performance Status (PS) score ≤2 at enrollment
4. Provide written informed consent prior to any study-related procedure. (In the event that the patient is re-screened for study participation or a protocol amendment alters the care of an ongoing patient, a new informed consent form must be signed.)
5. Patients with a life expectancy of ≥3 months (90 days)
6. Patients with adequate major organ functions meeting the following criteria on the basis of laboratory data within 4 weeks (28 days) before enrollment (if multiple data are available, most recent data during the period):
a. Serum creatinine: ≤1.8× the upper limit of the normal (ULN) range
b. Total bilirubin: G2× the ULN
c. Aspartate transaminase (AST) and alanine transaminase (ALT): 3× the ULN
7. Be able to comply with the requirements of the entire study.

### Exclusion Criteria:

Patients meeting any one of these exclusion criteria will be prohibited from participating in the study.
1. Presence of concomitant severe cardiovascular disease:
a. Patients who had myocardial infarction within 6 months (180 days) before enrollment
b. Patients with significant diseases at enrollment that may affect study treatment, such as New York Heart Association (NYHA) Functional Class III or IV heart disease, National Cancer Institute (NCI) Common Terminology Criteria for Adverse Events (CTCAE) v5.0 grade ≥3 arrhythmia, angina pectoris, abnormal electrocardiogram findings, interstitial pneumonia or pulmonary fibrosis
2. Presence of concomitant malignancy requiring chemotherapy or any malignancy (except basal and squamous cell carcinoma of the skin) for which the patient received chemotherapy within 6 months prior to enrollment. NOTE: Diagnosis of any previous or concomitant malignancy is thus not an exclusion criterion.
3. Presence of uncontrolled or uncontrollable infection(s); or ≥Grade 3 infection according to NCI CTCAE v5.0
4. Presence of any psychological, familial, sociological or geographical condition that, in the opinion of the investigator, could potentially hinder compliance with the study protocol and follow-up schedule
5. Patients with a dry tap on bone marrow aspiration before enrollment
6. Patients with concurrent autoimmune disease or a history of chronic or recurrent autoimmune disease, or patients who require long-term systemic steroid therapy greater than the equivalent of 20 mg of prednisone daily (excluding therapy given on an ‘as needed’ [PRN] basis)
7. Patients with other documented malignancies within past year aside from synchronous or metachronous multiple cancers with a disease-free period of ≤5 years (excluding carcinoma in situ, mucosal carcinoma, or other such carcinomas curatively treated with local therapy)
8. Patients with >Grade 2 hemorrhage according to NCI CTCAE v5.0
9. Patients who have previously received alvocidib or another cyclin-dependent kinase 9 (CDK9) inhibitor
10. Patients who are pregnant or breastfeeding
11. Female patients of childbearing potential who are sexually active and unwilling to use a medically acceptable method of contraception associated with a low failure rate during and for at least 6 months after the last dose of study drug (Patients will be considered to be of childbearing potential unless surgically sterilized by hysterectomy, or bilateral tubal ligation/salpingectomy, or postmenopausal for at least 2 years.)
12. Male patients with partners of childbearing potential who are unwilling to use condoms in combination with a second effective method of contraception during the trial and for at least 6 months after the last administration of study treatment.
13. Patients who are inappropriate for participation in the study for other reasons in the opinion of the investigator or sub-investigator(s)
14. Patients with a known hypersensitivity to DEC, AZA or mannitol EXAMPLE 10: treatment protocols (DEC+alvocidib)

Patients receive alvocidib administered in sequence after DEC or AZA. Decitabine (DEC) is administered as a 1-hour intravenous (IV) infusion (IVI) daily for 5 days at a dose of 20 mg/m² followed on Day 8 by alvocidib as a loading dose over 30-minutes followed by a 4-hour IVI according to the following schedule.

TABLE 4
Decitabine + alvocidib dosing

| Dose | Days 1-5, Decitabine | Day 8, Alvocidib ^{b} | |
|---|---|---|---|
| Level^{a} | 1-hr IV infusion | 30-min bolus | 4-hr IV infusion |
| 1 | 20 mg/m² | 20 mg/m² | 20 mg/m² |
| 2 | 20 mg/m² | 30 mg/m² | 30 mg/m² |
| 3 | 20 mg/m² | 30 mg/m² | 45 mg/m² |
| 4 | 20 mg/m² | 30 mg/m² | 60 mg/m² |

^{a}It is possible for additional and/or intermediate dose levels to be added during the course of the study.
^{b} Alvocidib to be administered first as a 30-minute (±10 minutes) IV bolus followed up to 30 minutes later by a 4-hour (±15 minutes) IVI.
^{c} Once Cohort 4 is completed using the hybrid dosing schedule (ie, 30-min IV bolus + 4-hr IVI), a cohort of at least 3 patients will receive DEC followed by 90 mg/m² of ALV administered by 1-hr IVI.

Once the MTD of alvocidib administered via hybrid dosing (e.g., 30-minute bolus followed by a 4-hour IVI) has been determined, 2 cohorts of patients (minimum of 6 patients; 3 per cohort) will receive AZA followed by alvocidib administered as a 1-hour IVI. The dose of alvocidib in the first AZA cohort will be 75 mg/m². In the absence of any significant toxicities, the alvocidib dose will be escalated.
Days 1-5: Decitabine to be administered at a starting dose of 20 mg/m² as a 1-hour IVI infusion (ie, starting dose for Cohort 1; see Table 4 (above) for assigned decitabine doses per treatment cohort).
Day 6 and 7: Drug-free days
Day 8: Alvocidib to be administered at a starting dose of 20 mg/m² as a 30-minute (±10 minutes) IV bolus followed up to 30 minutes later by 30 mg/m² administered as a 4-hour (±15 minutes) IVI (starting dose for Cohort 1; see Table (above) for assigned alvocidib doses per treatment cohort).

### Example 11: Treatment Protocols (AZA+Alvocidib)

Azacitidine may be administered on either a 7-day schedule (e.g., 7 consecutive days) or a 5-2-2 schedule (e.g., once daily for 5 days followed by 2 drug-free days with 2 more days of treatment. Azacitidine may be given as an IVI over 10 to 40 minutes or as a subcutaneous (SC) injection. Regardless of which AZA schedule or route of administration is used, alvocidib will be given on Day 10 as a 1-hour IVI. Choice of schedule and route of administration of AZA will be at the discretion of the investigator.

TABLE 4A
Azacitidine + alvocidib dosing

| Dose | Azacitidine ^{b} | Day 10, Alvocidib |
|---|---|---|
| Level ^{a} | IVI or SC injection ^{c} | 1-hr IVI |
| 1A | 75 mg/m² | 75 mg/m² |
| 1B | 75 mg/m² | 90 mg/m² |

^{a} It is possible for additional and/or intermediate dose levels to be added during the course of the study.
^{b} AZA can be administered on either a 7-day or 5-2-2 schedule.
^{c} AZA may be given as an IVI over 10 to 40 minutes or an SC injection.

### AZA 7-Day Schedule:

Days 1-7: AZA to be administered as a 75 mg/m² IV bolus over 10 to 40 minutes or SC injection daily for 7 consecutive days
Days 8-9: Drug-free days
Day 10: Alvocidib, 75 or 90 mg/m², to be administered as a 1-hour IVI
AZA 5-2-2 Schedule:
Days 1-5: AZA to be administered as a 75 mg/m² IV bolus over 10 to 40 minutes or SC injection daily for 5 consecutive days
Days 6-7: Drug-free days
Days 8-9: AZA to be administered as a 75 mg/m² IVI over 10 to 40 minutes or SC injection daily for 2 consecutive days
Day 10: Alvocidib, 75 or 90 mg/m², to be administered as a 1-hour IVI

In one embodiment, a short dose escalation of azacytidine will be used, starting at one dose lower than is being used with the decitabine combination, with the goal of escalating to 90 mg/m².

The alvocidib dose may be administered as a single 1 h infusion or as a hybrid bolus/infusion dosing schedule.

10 patients will be enrolled in Phase 2 to bring the total evaluable patients up to 25. The Phase 2 study will use the RP2D of alvocidib administered by 1-hour IVI from the Phase 1b study and follow a Simon 2-stage minimax design. Patients are eligible to receive a minimum of 4 cycles of treatment.

### Example 12: Supportive Care and TLS Prophylaxis

Supportive care measures for all patients to include:
Infection prevention (antibiotics, antifungals, antivirals) according to institutional standards
Routine growth factor support is not allowed. Growth factor support can be given at the discretion of the Investigator and with the Medical Monitor's approval in the presence of life threatening infection with ongoing neutropenia.
Donor lymphocyte infusions are not allowed at any time during the study

Tumor lysis may occur as part of initial cytoreductive therapy. The most extreme form, known as Tumor Lysis Syndrome (TLS), is characterized by hyperkalemia, hyperuricemia, hyperphosphatemia, increased lactate dehydrogenase (LDH), coagulopathy, and a potential cytokine release syndrome. Preventative measures to reduce the likelihood of developing TLS include ensuring adequate hydration of patients prior to administration of alvocidib as well as careful monitoring of laboratory parameters before and after infusion. Investigators should follow their own institutional protocols in determining the best treatment for patients with symptoms of TLS.
TLS Prophylaxis (DEC+ALV)
All patients receiving DEC will receive TLS prophylaxis as per each institution's standard of care
TLS Prophylaxis (AZA+ALV)

All patients receiving alvocidib in sequence following AZA will receive TLS prophylaxis prior to first dose of AZA as per each institution's standard of care.

Prior to the first dose of alvocidib, TLS prophylaxis will be instituted as detailed below:

### Example 13: Tumor Lysis Prevention and Treatment

Mandatory IV hydration with 0.45% NaCl (or similar hydration fluid per institutional standard) sterile solution at 500 cc for 1-2 hours prior to alvocidib, then an additional 500 cc for 1-2 hours after alvocidib during Cycle 1 (optional for subsequent cycles).

Replacement of excessive fluid losses, including from diarrhea, should be done unless otherwise clinically indicated, during AZA and alvocidib dosing.

Alvocidib may induce mild diarrhea during treatment days. Over-the-counter measures are typically effective in this setting if initiated early. It is strongly suggested that patients take 2 tablets of loperamide, 2 mg each (or equivalent), prior to the alvocidib IVI and then take 1 tablet (2 mg) for every loose stool up to a maximum of 8 tablets (16 mg) in a 24-hour period. Persistent diarrhea despite optimal outpatient management would trigger medical consultation. Early consideration should be given for possible Clostridioides difficile (C. diff) infection in this patient population and identifying/treating as expeditiously as possible should be top of mind.

Mandatory oral allopurinol to be started at least 72 hours prior to Day 10 of Cycle 1 and continued until completion of the first cycle (ie, 28 days). This may be discontinued for subsequent treatment cycles if uric acid levels are within normal limits and there is no evidence of tumor lysis syndrome.

Mandatory oral phosphate binder to be started at the same time as initiation of IV hydration on Day 10 of Cycle 1 and continued for the two weeks (i.e., 14 days).

If serum phosphorus levels are <3 after the first treatment with alvocidib and there is no evidence of TLS, phosphate binders may be discontinued. Patients should continue to be monitored for TLS as outlined for subsequent treatment cycles. Caution is warranted for patients who still have a high blast count as they remain at risk for TLS with subsequent treatments.

Evaluation of laboratory indicators of TLS during Cycle 1:

Tumor lysis laboratory evaluations (tumor lysis labs) include electrolytes (sodium, potassium, chloride, and carbon dioxide) as well as creatinine, calcium, lactate dehydrogenase (LDH), uric acid, and phosphorus levels.

During Cycle 1, monitor tumor lysis labs prior to first AZA and 2 hours (±30 minutes) after completion of first AZA dose. Monitor tumor lysis labs prior to alvocidib infusion and 2 hours (±30 minutes) after completion of IV hydration post alvocidib. All tumor lysis labs should be drawn, however the potassium level obtained at 2 hours post hydration should be reviewed immediately to determine if additional treatment is warranted. Labs will also be drawn daily for the first 2 days following alvocidib (i.e., Days 11-12) and at least weekly for the remainder of Cycle 1.

During Cycle 2, tumor lysis labs will be assessed prior to C2D1 first AZA dose and at 2 hours post C2D1 AZA dose, and prior to C2D10 alvocidib IVI and 2 hours (±30 minutes) after completion of alvocidib IVI.

During Cycles 3+, tumor lysis labs will be assessed at the discretion of the investigator based on blast counts

Suggested doses of these supportive care therapies are provided in the protocol; however, adjustment of the dosages based on the patient's clinical condition or each institution's standard of care is permitted.

### Example 14: PK Endpoints

Plasma concentrations of HMA and/or alvocidib will be summarized by descriptive statistics, including mean, n, standard deviation, coefficient of variation, minimum, maximum, and median. Prior to analysis of study samples, the assay sensitivity, specificity, linearity, and reproducibility will be documented.

Plasma PK analyses for alvocidib and known metabolites, if any, and dose proportionality will be determined on Days 8 and 9 (for patients receiving alvocidib following DEC) or Days 10 and 11 (for patients receiving alvocidib following AZA) of Cycle 1 in all patients enrolled in the study, according to Table 5 or Table 6:

TABLE 5
Cycle 1, Day 8 (DEC + ALV)/Cycle 1, Day 10 (AZA + ALV):

| PK | | | |
|---|---|---|---|
| Sample | Timepoint in Relation to | | |
| No. | Alvocidib Administration | Window | |
| 1 | Prior to IVI | At any time | |
| 2 | End of IVI | ±10 | mins |

Up to 30-min wait (±10 minutes) ^{a}

| | | | |
|---|---|---|---|
| 3 | 30 mins after end of IVI | ±10 | mins |
| 4 | 1 hr after end of IVI | +10 | mins |
| 5 | 2 hrs after end of IVI | ±15 | mins |
| 6 | 4 hrs after end of IVI | ±15 | mins |

^{a} This wait time is only required in patients receiving DEC followed by alvocidib hybrid dosing (ie, 30-min IV bolus; up to 30-min wait; and then the 4-hr IVI).

TABLE 6
Cycle 1, Day 9 (DEC + ALV)/Cycle 1, Day 11 (AZA + ALV):

| PK | | |
|---|---|---|
| Sample | Timepoint in Relation to | |
| No. | Alvocidib Administration | Window |
| 7 | After alvocidib IVI | ±1 hr |
| | (23 hrs after start of 4-hr IVI in pts | |
| | receiving hybrid dosing or end of | |
| | infusion in pts receiving 1-hr IVI) | |

### Example 15: PD Endpoints

Any possible correlation between the rate of CR/CRi/CRmarrow/PR/HI and BH3 profiling will be determined by flow cytometry with an emphasis on MCL-1 dependence.

The types of biomarkers to be analyzed may include, but are not limited to, nucleic acids, proteins, lipids or metabolites. Biomarker assessments may be used to assess and generate prognostic, predictive, or surrogate biomarker signatures. These assessments may be explored in the context of MDS or related conditions or drugs of similar class. The results from these analyses are exploratory in nature and may not be included in a clinical study report (CSR). Samples may be analyzed according to schedules provided in Table 7 or Table 8.

TABLE 7
DEC + ALV PD Sample Collection Schedule

| PD | | | | |
|---|---|---|---|---|
| Sample | | Timepoint in Relation to | | |
| No. | Visit/Day | Drug Administration | Window | |
| 1 | Screening | Day −28 to Day 1 | At any time | |
| 2 | C1D1 | Prior to DEC infusion | ≤10 mins prior | |
| 3 | C1D3 | Prior to DEC infusion | ≤10 mins prior | |
| 4 | C1D5 | Prior to DEC infusion | ≤10 mins prior | |
| 5 | C1D8 | Prior to alvocidib IVI | At any time | |
| 6 | C1D9 | After alvocidib IVI | Within ±1 hr | |
| | | (23 hrs after start of 4-hr IVI | | |
| | | in pts receiving hybrid dosing | | |
| | | or end of infusion for pts | | |
| | | receiving 1-hr IVI) | | |
| 7 | C1D15 | During Study Visit | ±3 | days |
| 8 | C1D22 | During Study Visit | ±3 | days |
| 9 | C2D1 | Prior to DEC infusion | At any time | |
| 10 | C2D8 | Prior to alvocidib IVI | At any time | |
| 11 | C2D15 | During Study Visit | ±3 | days |
| 12 | C2D28 | During Study Visit | +1/−3 | days |

| | | | |
|---|---|---|---|
| 13 | C4* + | During Study Visit | At any time |
| | D28 | | |
| 14 | EOS | During Study Visit | At any time |

*Even cycles beginning with Cycle 4.

TABLE 8
AZA + ALV PD Sample Collection Schedule

| PD | | | | |
|---|---|---|---|---|
| Sample | | Timepoint in Relation to | | |
| No. | Visit/Day | Drug Administration | Window | |
| 1 | Screening | Day −28 to Day 1 | At any time | |
| 2 | C1D1 | Prior to AZA infusion | ≤10 mins prior | |
| 3 | C1D3 | Prior to AZA infusion | ≤10 mins prior | |
| 4 | C1D5 | Prior to AZA infusion | ≤10 mins prior | |
| 5 | C1D7 | Prior to AZA infusion | ≤10 mins prior | |
| 6 | C1D10 | Prior to alvocidib IVI | At any time | |
| 7 | C1D11 | After alvocidib IVI | Within ±1 hr | |
| | | (23 hrs after end of infusion) | | |
| 8 | C1D15 | During Study Visit | ±3 | days |
| 9 | C1D22 | During Study Visit | ±3 | days |
| 10 | C2D1 | Prior to AZA infusion | At any time | |
| 11 | C2D10 | Prior to alvocidib IVI | At any time | |
| 12 | C2D15 | During Study Visit | ±3 | days |
| 13 | C2D28 | During Study Visit | +1/−3 | days |

| | | | |
|---|---|---|---|
| 14 | C4* + | During Study Visit | At any time |
| | D28 | | |
| 15 | EOS | During Study Visit | At any time |

*Even cycles beginning with Cycle 4.

Additional exploratory analyses may be performed if useful in the interpretation of the data and/or to assist the sponsor in planning future studies.

### Example 16: In Vitro Pharmacodynamic Marker Study on CD34⁺ MDS Bone Marrow Mononuclear Cells (BMMNC)

A pharmacodynamic maker study was performed for azaditidine and alvocidib using MDS patient-derived BMMNC from Cureline. CD34⁺ cells were isolated by CD34 MicroBead Kit (Miltenyi Biotec) and cultured in StemSpan™ SFEM II with StemSpan™ CD34⁺ Expansion Supplement (STEMCELL Technologies). Cells were seeded in 6-well plates and treated with DMSO or azacitidine (0.3 or 0.6 μM) for 24 hours. Following that, DMSO or alvocidib (0.1 μM) was sequentially added, and the cells were incubated for an additional 6 hours. Protein was extracted using RIPA buffer with protease and phosphatase inhibitor cocktail. Protein expression was assessed by Western blot using NOXA (CST, Cat #14766S), MCL-1(CST, Cat #392245) and β-actin (CST, Cat #4967L) antibodies. Apoptotic activity was assessed by Caspase-Glo® 3/7 assay (Promega).

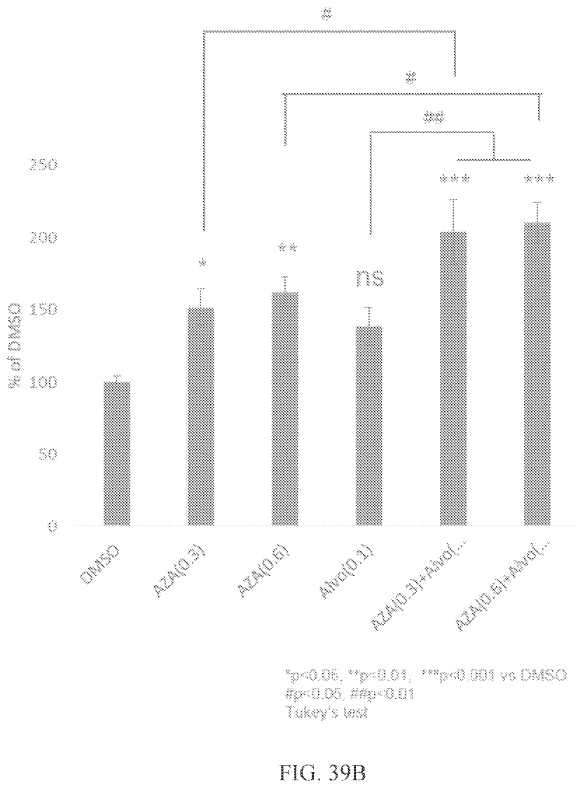

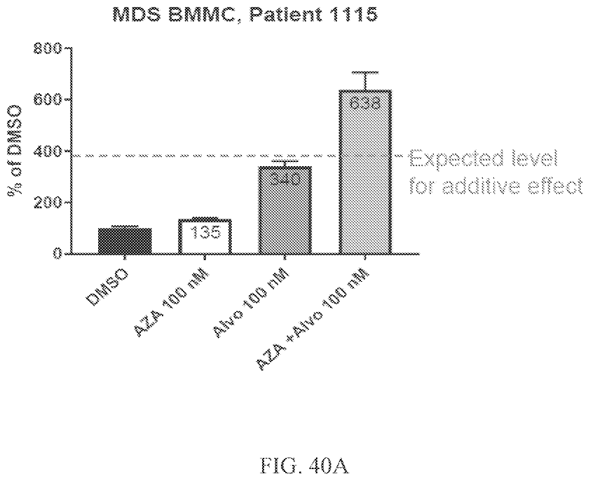

FIG. 39A shows that azacitidine (alone) induced NOXA expression and alvocidib (alone) suppressed MCL-1 expression, whereas sequential treatment of azacitidine and alvocidib showed both NOXA induction and MCL-1 suppression compared with a DMSO-treated sample in CD34⁺ MDS BMMNC from Patient 0219. FIG. 39B shows that azacitidine (alone) showed 52 and 62% increase in apoptotic activity at the concentration of 0.3 and 0.6 μM, respectively, whereas sequential treatment of azacitidine (0.3 or 0.6 μM) and alvocidib showed 104 and 110% increase of apoptotic activity, respectively, compared with a DMSO-treated sample, and was statistically significant compared with either of the single treatment samples. Sequential treatment of azacitidine and alvocidib modulates apoptotic pathway by apoptotic protein induction and anti-apoptotic protein reduction in CD34⁺ MDS patient derived BMMNC.

### Example 17: In Vitro Efficacy Study on CD34⁺ MDS Bone Marrow Mononuclear Cells (BMMNC)

An efficacy study was performed for azacitidine and alvocidib using MDS patient-derived BMMNC from Cureline. CD34⁺ cells were isolated by CD34 MicroBead Kit (Miltenyi Biotec) and cultured in StemSpan™ SFEM II with StemSpan™ CD34⁺ Expansion Supplement (STEMCELL Technologies). Cells were seeded in 96-well plates and treated with DMSO or azacitidine (100 nM) for 24 hours. Following that, DMSO or alvocidib (100 nM) was added, and the cells were incubated for additional 24 hours. Apoptotic activity was assessed by Caspase-Glo® 3/7 assay (Promega).

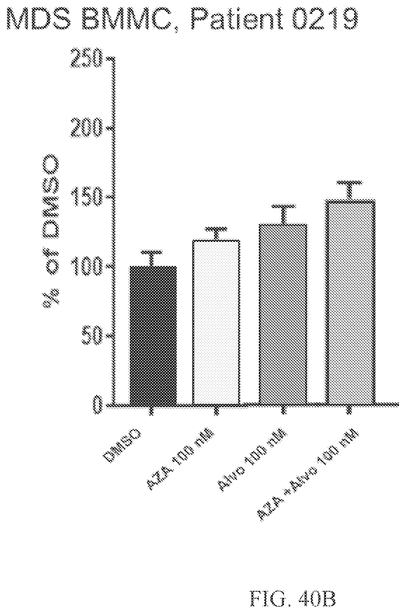

FIG. 40A shows that azacitidine (alone) and alvocidib (alone) showed 35% and 240% increase of apoptotic activity, respectively, whereas sequential treatment of azacitidine and alvocidib showed 538% increase of apoptotic activity compared with a DMSO-treated sample in CD34⁺ MDS BMMNC from Patient 1115, which was statistically significant compared with either of the single treatment samples. Patient 1115 had high-risk MDS.

FIG. 40B shows that azacitidine (alone) and alvocidib (alone) showed 20% and 31% increase of apoptotic activity, respectively, whereas sequential treatment of azacitidine and alvocidib showed 49% increase of apoptotic activity compared with a DMSO-treated sample in CD34⁺ MDS BMMNC from Patient 0219, which was not statistically significant compared with either of the single treatment samples.

Sequential treatment of alvocidib following azacitidine shows synergistic apoptosis induction in CD34⁺ MDS patient-derived BMMNC.

### Example 18: T-MS1 Based MCL-1 Dependency Assay

To determine the patients' cells' dependency on MCL-1 anti-apoptotic protein for survival, the MCL-1 binding protein, MS I with N-terminal modifications for improved cell penetrance was utilized. The modified peptide is referred to as T-MS1. When added to cells, T-MS1 crosses the plasma membrane and antagonizes MCL-I, leading to mitochondrial outer membrane pore (MOMP) formation and subsequently, depolarization of the mitochondria. Mitochondrial potential was assessed using the cationic dye, DiOC₂(3), which accumulates in negatively-charged, healthy mitochondria. Fresh or previously frozen MDS bone marrow cells were treated with azacitidine or DMSO control in media containing RPMI+10% FBS+pen/strep for 48 hours prior to being interrogated with T-MS1. After blocking and staining with antibody cocktail to gate on MDS blasts, T-MS1 was added and cells were incubated for 30 minutes at 37° C. After T-MS1 treatment, cells were washed and stained with cationic dye, Dioc6, for 90 minutes to assess mitochondrial depolarization via flow cytometric analysis. Priming % was calculated using the formula:

$$Priming \% = ( Control - Treatment ) / Control * 100.$$

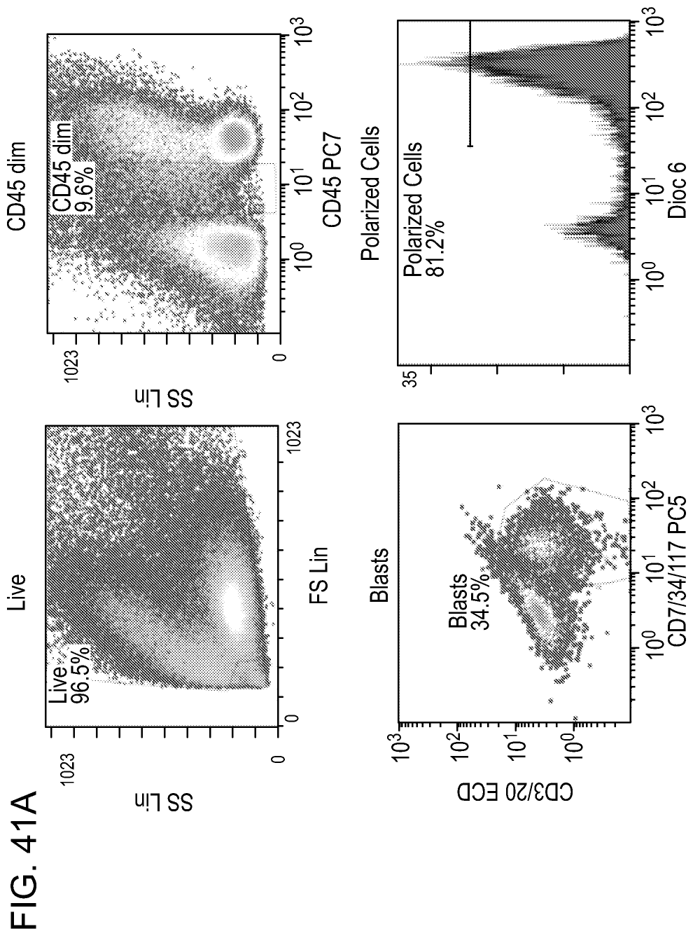

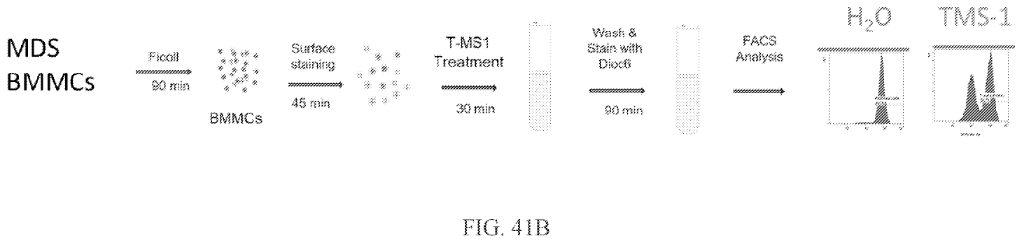

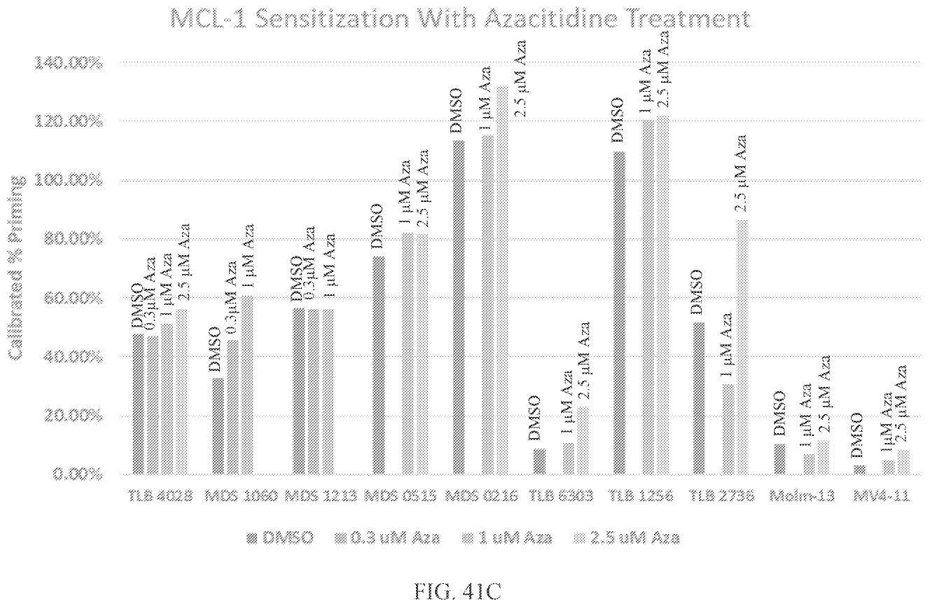

For concordance with prior priming values, an additional calibration factor of 1.6 was applied to patient priming values. FIG. 41A shows the gating strategy employed for this assay, and FIG. 41B shows the assay design.

To determine the patients' cells' MCL-1 dependency following treatment with azacitidine, MDS patient-derived BMMCs were treated with DMSO, 0.3 μM azacitidine, 1 μM azacitidine or 2.5 μM azacitidine for 48-72 hours before being assessed by the MCL-1 dependency assay. FIG. 41C shows MCL-1 sensitization of several patients' blasts upon treatment with azacitidine. Patient 0216's blasts showed a percent priming of 113.4% for DMSO control, 115.3% for the 1 μM azacitidine treatment and 132.1% for the 2.5 μM azacitidine treatment. Patient 0515's blasts showed a percent priming of 74.1% for DMSO control, 82.1% for the 1 μM azacitidine treatment and 81.6% for the 2.5 μM azacitidine treatment. Patient 1060's blasts showed a percent priming of 32.7% for DMSO control, 45.8% for the 72-hour, 0.3 μM azacitidine treatment and 60.9% for the 72-hour, 1 μM azacitidine treatment.

There was a dose-dependent increase in priming observed with 0.3, 1 and 2.5 μM azacitidine treatment across multiple bone marrow samples from patients with MDS. Priming increased in nearly all the patient samples, particularly at 1 and 2.5 μM azacitidine, with a maximum increase of 35% in one patient sample.

### Example 19: Interim Clinical Trial Results

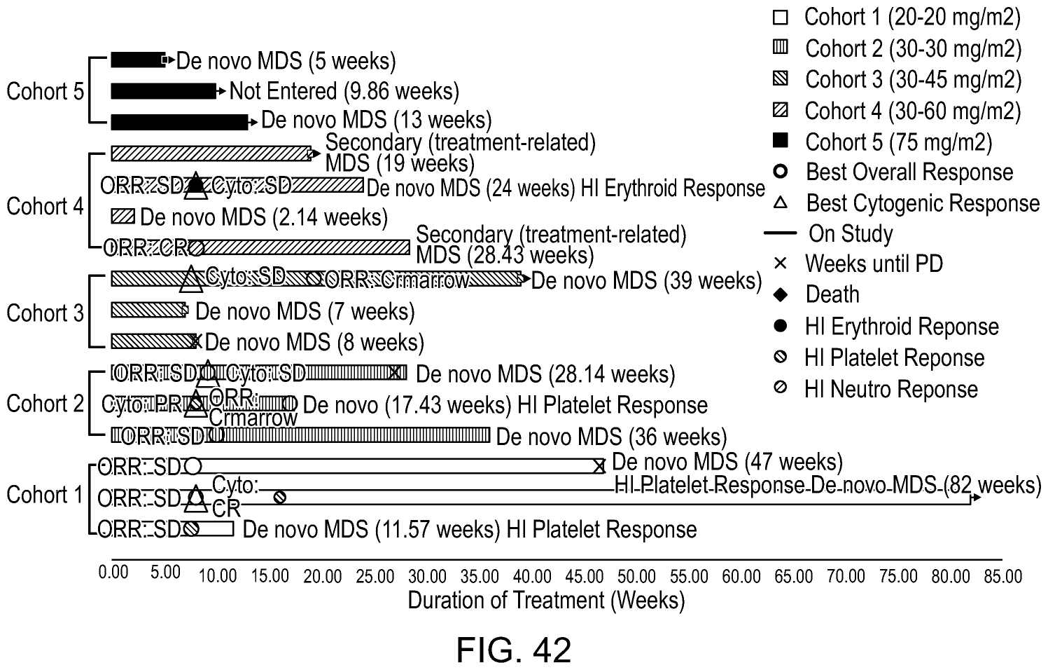

A clinical trial was conducted in accordance with the guidelines described in Examples 9-15. Interim results from the clinical trial are depicted in FIG. 42.

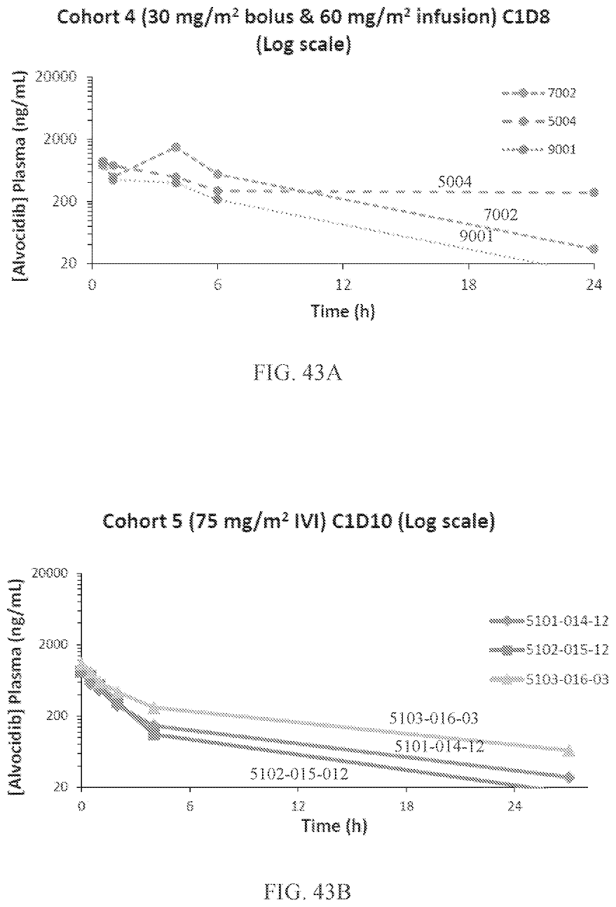

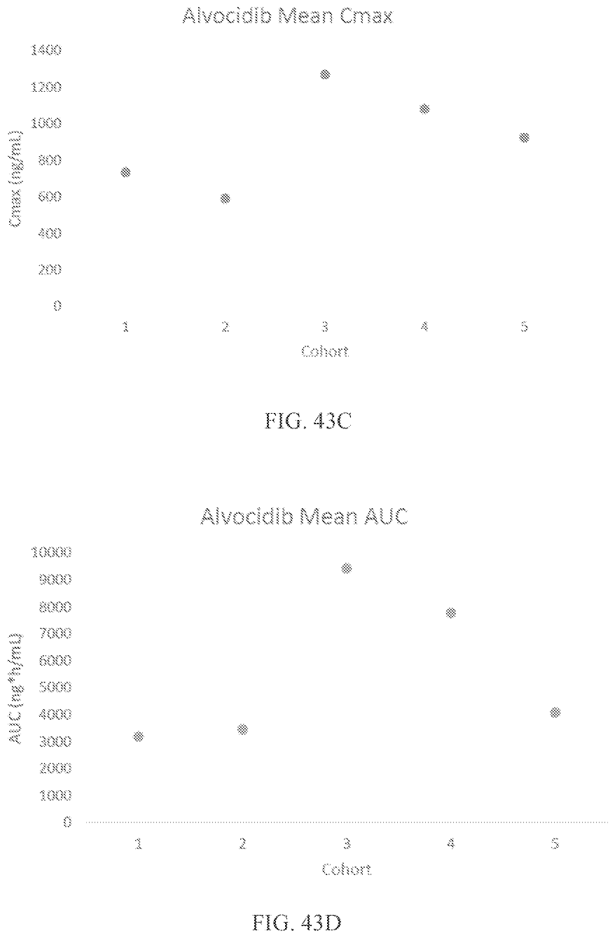

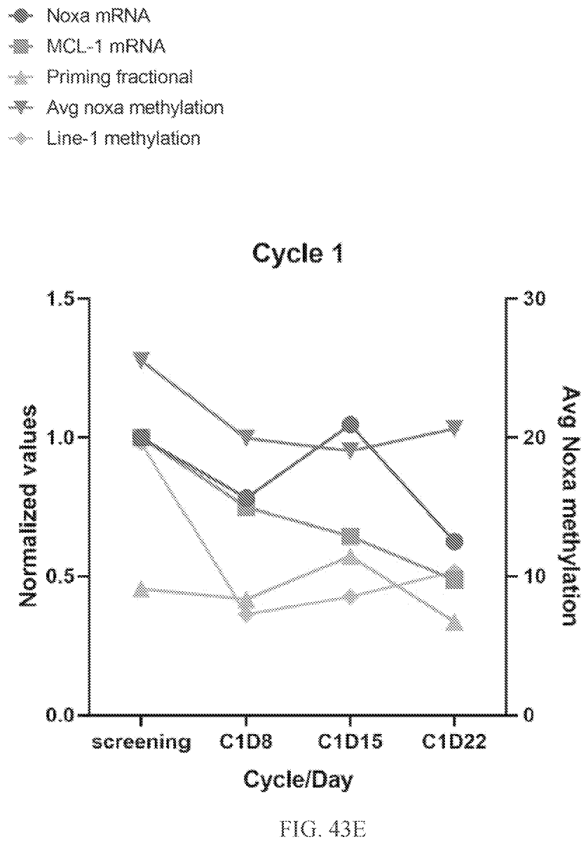

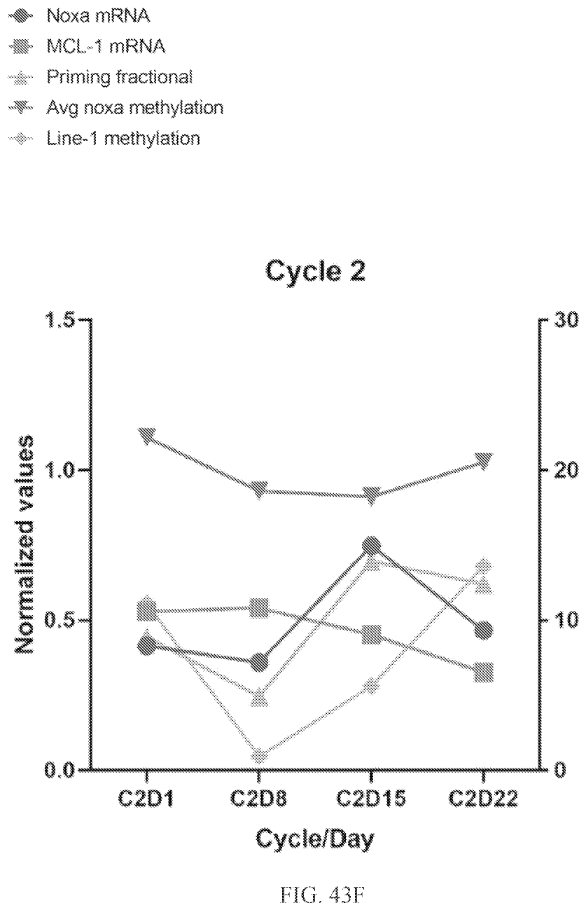

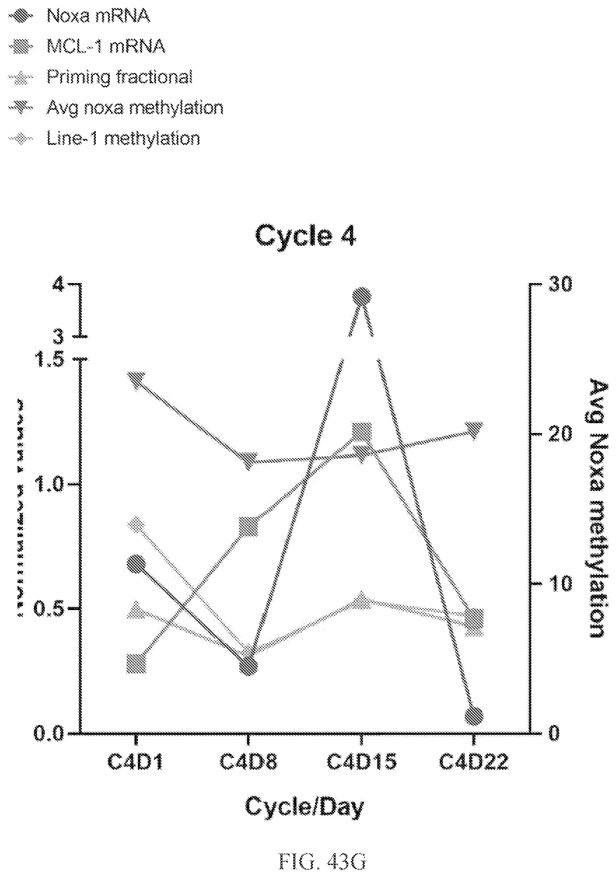

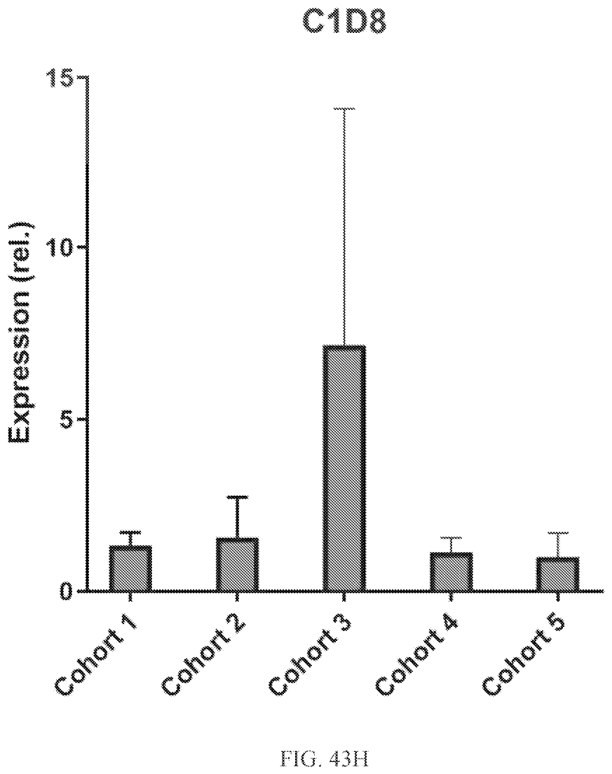

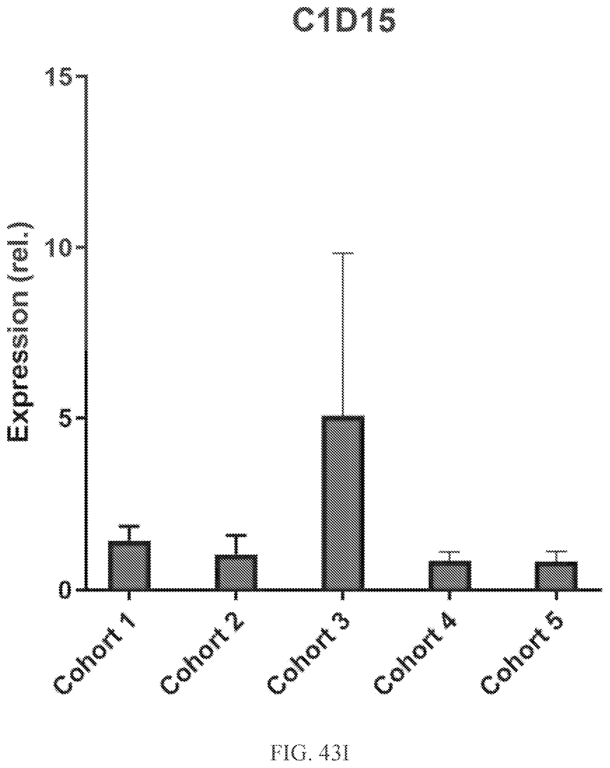

FIGS. 43A and 43B show pharmacokinetic curves for alvocidib from cohort 4 (30 mg/m² bolus+60 mg/m² infusion) on C1D8, and cohort 5 (75 mg/m² IVI) on C1D10. A summary of pharmacokinetic data calculated from the curves in FIGS. 43A and 43B appears in Tables 9 and 10, respectively.

TABLE 9
Cohort 4 (30 mg/m² bolus + 60 mg/m² infusion)

| Cohort 4 | Cₘₐₓ | Tₘₐₓ | AUC(0-24) | T_{1/2} |
|---|---|---|---|---|
| Subject # | (ng/mL) | (h) | (ng*h/mL) | (h) |
| 7002 | 1490 | 4 | 10800 | 3.97 |
| 5004 | 877 | 0.5 | 8470 | — |
| 9001 | 880 | 0.5 | 4080 | 4.71 |
| Mean | 1082 | 1.7 | 7783 | 4.3 |
| SD | 353 | 2.0 | 3412 | 0.5 |
| CV | 33% | 121% | 44% | 12% |

TABLE 10
Cohort 5 (75 mg/m² IVI)

| Cohort 5 | Cₘₐₓ | Tₘₐₓ | AUC(0-27) | T_{1/2} |
|---|---|---|---|---|
| Subject # | (ng/mL) | (h) | (ng*h/mL) | (h) |
| 5101-014-12 | 868 | 0 | 3410 | 8.23 |
| 5102-015-12 | 835 | 0 | 3030 | 6.74 |
| 5103-016-03 | 1070 | 0 | 5800 | 10.0 |
| Mean | 924 | 0 | 4080 | 8 |
| SD | 127.2 | 0.0 | 1501.6 | 1.6 |
| CV | 13.8% | | 36.8% | 19.6% |

The Cₘₐₓ decreased by 15.4% going from cohort 4 to cohort 5, and AUC decreased by 47.6% going from cohort 4 to cohort 5. The AUC observed in cohort 5 reflects the shorter duration of treatment administration utilized in cohort 5. In cohort 5, the observed T_{1/2} was 8 hours.

FIGS. 43C and 43D show the mean Cₘₐₓ and AUC, respectively, of alvocidib by cohort. Relative to cohort 4, an increase in Cₘₐₓ and AUC were not observed in cohort 5. FIGS. 43E, 43F and 43G show various treatment trends for cohorts 1-4 as a function of treatment cycle. FIGS. 43H and 43I show relative NOXA expression on C1D8 and C1D15, respectively, as a function of cohort.

### Example 20: BH3 Profiling Assay

The BH3 profiling assay is done according to T-MS1-based MCL-1 dependency assay protocol with minor modifications. Briefly, cells (e.g., BMMC) grown in culture or frozen archival samples are stained with antibody cocktail after recovery at 37° C. for 60 minutes in media containing RPMI+10% FBS+pen/strep. 250,000 cells per condition are treated for 45 min at 37° C. with T-PUMA, T-BAD, T-MS1, and T-HRK in duplicate, or with vehicle alone as negative control. Assay is performed along with inhibitors to elucidate exact mechanism of T-BAD peptide-dependence membrane depolarization. Inhibitor and BH3 peptide are added and incubated for 30 minutes at 37° C. There is no specific BCL-W inhibitor—therefore, possible BCL-W dependency is determined by the cell's dependency on T-BAD. After inhibitor/peptide treatment, cells are washed and stained with cationic dye, Dioc6, for 90 min to assess mitochondrial depolarization via flow cytometric analysis. Priming % is calculated using the formula:

$$Priming \% = ( Control - Treatment ) / Control * 100.$$

A calibration factor of 1.6 is applied to T-MS1 priming result to stay consistent with priming percentage obtained from T-MS1 Based MCL-1 Dependency Assay. T-MS1 is reconstituted in H₂O. T-PUMA, T-BAD, and T-HRK are reconstituted in 30% TFE (trifluoroethanol) to maintain correct alpha-helical conformation of the peptide for specific binding and activity. Inhibitors are reconstituted in DMSO.

### Example 21: Form B

Data was acquired according to the parameters listed below:

X-ray powder diffraction (XRPD): Stoe Stadi P. Copper KαI radiation, 40 kV/40 mA; Mythen 1K detector transmission mode, curved monochromator, 0.02° 2θ step size, 12 s step time, 1.5-50.5° 2θ scanning range with 1° 2θ detector step in step-scan mode. Each sample (25-40 mg of powder) was placed between two cellulose acetate foils spaced with a metal washer (0.4 mm thick, 12-mm inner diameter; “sandwich element”). The sandwich element was transferred to a sample holder (SCell) that was sealed with acetate foils. Samples were acquired in ambient air atmosphere and rotated during measurements

TG-FTIR: Netzsch Thermo-Microbalance TG 209 with Bruker FT-IR Spectrometer IFS28 or Vector 22; Al crucible with microhole, N₂ atmosphere, 10 K/min heating rate, 25° C. to 300° C. (or 350° C. range).

HPLC: The method used to detect and determine purity of compound of structure (I) and related substances (such as alvocidib) was a reverse-phase HPLC method with a gradient program and DAD detection technique. Reverse phase C₁₈ Waters X-bridge 150 mm×4.6 mm, 3.5-μm particle column; flow rate=1.0 mL/min; detection wavelength=265 nm; run time: 35.0 minutes; sample diluted in methanol; mobile phase A was 80:20 (v/v) pH 6.5 buffered aqueous acetonitrile; mobile phase B was 35:65 (v/v) pH 6.5 buffered aqueous acetonitrile; 1.0 mL/min; column temperature=35° C. The gradient program is depicted in the following table:

| Run Time (minutes) | Mobile Phase A (%) | Mobile Phase B (%) |
|---|---|---|
| 0 | 100 | 0 |
| 8 | 100 | 0 |
| 16 | 66 | 34 |
| 24 | 0 | 100 |
| 26 | 0 | 100 |
| 26.5 | 100 | 0 |
| 35 | 100 | 0 |

Total Impurities (%) was calculated by summing the percentages of each individual impurity, including alvocidib. Other Impurities (%) was calculated by summing the percentages of each individual impurity, excluding alvocidib. Purity of compound of structure (I) (%) was calculated by taking the difference between 100% and the Total Impurities (%). All individual impurities at and above 0.05% were taken for the calculation of total impurities.

¹H NMR: Bruker DPX 300 using a frequency of 300.13 MHz, a 30° excitation pulse and a recycle delay of 1 s. 16-1024 scans were accumulated per spectrum; deuterated DMSO or D₂O was used as a solvent. Two-dimensional COSY spectra were acquired with 512 data points in the indirect dimension, an indirect time increment of 441.60 ps, 16 scans per slice and a recycle delay of 0.36 s.

Differential Scanning Calorimetry (DSC): DSC was performed using a TA Q200/Q2000DSC from TA Instruments using a ramp method and a crimped, aluminum sample pan at 25° C. The heating rate was 10° C./minute, and the purge gas was nitrogen.

Polymorph syntheses:

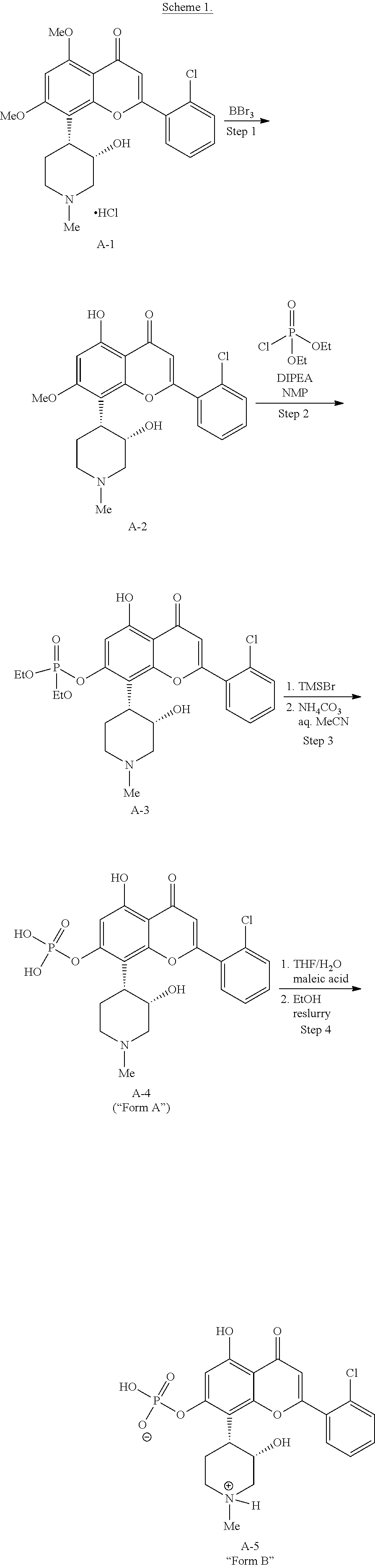

Step 1: A-1 was treated with boron tribromide in chlorobenzene. Removal of byproducts by distillation and crystallization from chlorobenzene-methanol-water resulted in A-2 as free base.
Step 2: A-2 (in free base form) was treated with diethyl chlorophosphate and diisopropylethylamine in N-methylpyrrolidone. Water was added to stop the reaction and precipitate the product. The resulting slurry was filtered, washed with water, and dried under vacuum to produce the compound A-3.
Step 3: A-3 was treated with trimethylsilyl bromide to deprotect A-3 and afford A-4 as a hydrobromide, which was treated with ammonium bicarbonate solution. The precipitated A-4 (i.e., compound of structure (I) having an amorphous crystal structure (i.e., “Form A”) was filtered and dried.
Step 4: A-4 was suspended in a mixture of THF and water (19:1), and maleic acid was added. After stirring at room temperature, the solid was filtered and dried in a filter dryer to afford A-5. The resultant filtered solid compound A-5 was suspended in ethanol and re-slurried before an additional filtration. Filtered product (i.e., A-5 as polymorph form B) was washed with ethanol and dried to afford the desired product.

Synthesis of polymorph Form B according to Scheme 1 has been conducted using 5.72 kg A-1, 4.17 kg A-2 and 2.70 kg A-3. At this scale, the yield of Step 1 was 79.9%. After recrystallization, the yield of A-3 from Step 2 was 48%, and the purity of compound A-3 thus obtained was 86% by HPLC. The combined yield of Steps 3 and 4 was 37.5%. The overall yield of the process of Scheme 1 was 15%, and the process yielded 0.90 kg of A-5.

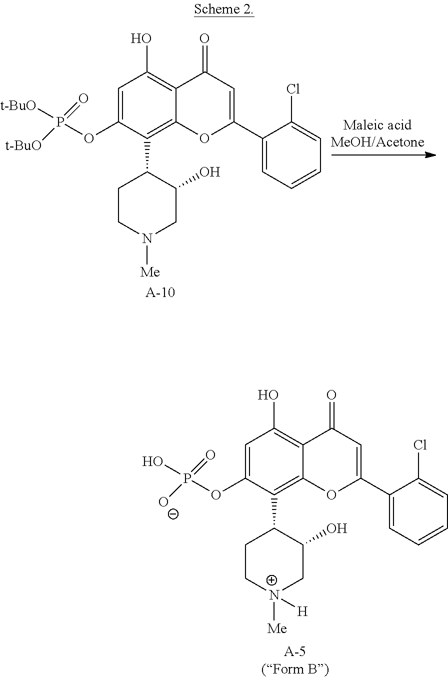

A-10 was obtained according to the protocol described in International Publication No. WO 2020/117988. A-10 (100 mg) was substantially dissolved in methanol (1 ml) at 50° C. A solution of maleic acid (12.2 mg, 0.5 equiv) in methanol (1.5 ml) was added dropwise to the mixture of A-10 in methanol, followed by acetone (2.5 ml). The resulting reaction mixture was stirred for one hour at room temperature, and then filtered to obtain A-5 (77.4 mg) having a crystallinity of 94%. Residual maleic acid was confirmed by ¹H NMR (0.01 proton).

Polymorph Form B was analyzed using XRPD and DSC using the parameters described above. The resultant spectra are shown in FIGS. 44A and 44B, respectively. Tabulated XRPD data generated for Form B is shown in Table 11.

TABLE 11
Tabulated data from XRPD diffractogram of Form B

| D value | | Intensity | Intensity | |
|---|---|---|---|---|
| (Å) | 2θ | (relative) | (absolute) | FWHM |
| 18.382645 | 4.8032 | 55.17 | 3833 | 0.08 |
| 9.190754 | 9.6155 | 10.48 | 728 | 0.08 |
| 8.157735 | 10.8365 | 93.39 | 6489 | 0.06 |
| 6.471747 | 13.6717 | 49.51 | 3440 | 0.08 |
| 5.956605 | 14.8604 | 73.17 | 5084 | 0.04 |
| 5.524677 | 16.0296 | 11.07 | 769 | 0.06 |
| 5.281129 | 16.774 | 30.9 | 2147 | 0.06 |
| 5.172326 | 17.1295 | 9.19 | 639 | 0.06 |
| 5.080202 | 17.4425 | 17.69 | 1229 | 0.06 |
| 4.984581 | 17.7798 | 58 | 4030 | 0.04 |
| 4.722631 | 18.7747 | 18 | 1250 | 0.06 |
| 4.654062 | 19.0539 | 25.93 | 1802 | 0.04 |
| 4.552517 | 19.483 | 32.41 | 2252 | 0.06 |
| 4.445879 | 19.955 | 100 | 6948 | 0.06 |
| 4.144506 | 21.4226 | 19.68 | 1367 | 0.04 |
| 4.079254 | 21.7694 | 22.77 | 1582 | 0.06 |
| 3.90758 | 22.7383 | 9.77 | 679 | 0.04 |
| 3.867711 | 22.9759 | 28.61 | 1988 | 0.06 |
| 3.72577 | 23.8638 | 22.65 | 1573 | 0.06 |
| 3.6868 | 24.1198 | 25.68 | 1785 | 0.04 |
| 3.616616 | 24.5951 | 48.94 | 3401 | 0.06 |
| 3.512733 | 25.3344 | 7.74 | 538 | 0.06 |
| 3.394705 | 26.2307 | 28.77 | 1999 | 0.06 |
| 3.350985 | 26.5791 | 11.43 | 794 | 0.04 |
| 3.319773 | 26.8337 | 18.57 | 1290 | 0.06 |
| 3.241092 | 27.4977 | 23.54 | 1635 | 0.08 |
| 3.142734 | 28.376 | 15.33 | 1065 | 0.06 |
| 3.055085 | 29.208 | 12.7 | 882 | 0.08 |
| 2.973274 | 30.0303 | 10.91 | 758 | 0.14 |
| 2.920812 | 30.5827 | 6.32 | 439 | 0.06 |
| 2.830324 | 31.5856 | 9.24 | 642 | 0.08 |
| 2.748258 | 32.5546 | 6.82 | 474 | 0.04 |
| 2.731945 | 32.7544 | 11.76 | 817 | 0.06 |
| 2.703955 | 33.1032 | 6.09 | 423 | 0.06 |
| 2.64826 | 33.8201 | 10.18 | 707 | 0.04 |
| 2.640271 | 33.9255 | 14.93 | 1037 | 0.06 |
| 2.60744 | 34.3659 | 16.09 | 1118 | 0.06 |
| 2.587021 | 34.6457 | 11.27 | 783 | 0.06 |
| 2.540227 | 35.3046 | 5.6 | 389 | 0.06 |
| 2.424055 | 37.0566 | 5.24 | 364 | 0.06 |
| 2.216436 | 40.6738 | 7.03 | 488 | 0.06 |
| 2.125602 | 42.4942 | 10.93 | 760 | 0.06 |
| 2.078432 | 43.5072 | 4.98 | 346 | 0.14 |
| 2.042561 | 44.3113 | 12.61 | 876 | 0.1 |
| 2.008764 | 45.0975 | 5.76 | 400 | 0.08 |
| 1.950189 | 46.5303 | 5.84 | 406 | 0.14 |

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A method of treating myelodysplastic syndrome (MDS) in a patient in need thereof, comprising administering to the patient a therapeutically effective amount of a hypomethylating agent (HMA) followed by administering to the patient a therapeutically effective amount of alvocidib, or a prodrug thereof, or a pharmaceutically acceptable salt of the foregoing, wherein the patient has one or more of the following:
previously untreated MDS;
received fewer than six cycles of treatment with a hypomethylating agent;
de novo MDS; or
secondary MDS; and
wherein MCL-1 dependence of the MDS increases with administration of the HMA, and the MDS has MCL-1 dependence of greater than or equal to 40% upon administration of the alvocidib, or a prodrug thereof, or a pharmaceutically acceptable salt of the foregoing.

2-10. (canceled)

11. The method of claim 1, wherein the HMA is azacitidine, or a prodrug thereof, or a pharmaceutically acceptable salt of the foregoing, or decitabine, or a prodrug thereof, or a pharmaceutically acceptable salt of the foregoing.

12. The method of claim 11, wherein the HMA is azacitidine, or a prodrug thereof, or a pharmaceutically acceptable salt of the foregoing.

13. (canceled)

14. A method of treating myelodysplastic syndrome (MDS) in a patient in need thereof, comprising administering to the patient a therapeutically effective amount of azacitidine, or a prodrug thereof, or a pharmaceutically acceptable salt of the foregoing; and a therapeutically effective amount of alvocidib, or a prodrug thereof, or a pharmaceutically acceptable salt of the foregoing, wherein:
the azacitidine, or a prodrug thereof, or a pharmaceutically acceptable salt of the foregoing, is administered on days 1, 2, 3, 4, 5, 6 and 7, or on days 1, 2, 3, 4, 5, 8 and 9 of a 28-day treatment cycle; and
the alvocidib, or a prodrug thereof, or a pharmaceutically acceptable salt of the foregoing, is administered on day 10 of the 28-day treatment cycle,
wherein MCL-1 dependence of the MDS increases with administration of the azacitidine, or a prodrug thereof, or a pharmaceutically acceptable salt of the foregoing. and the MDS has MCL-1 dependence of greater than or equal to 40% upon administration of the alvocidib, or a prodrug thereof, or a pharmaceutically acceptable salt of the foregoing.

15. The method of claim 12, wherein
azacitidine, or a pharmaceutically acceptable salt thereof, is administered to the patient.

16-23. (canceled)

24. The method of claim 11, wherein a prodrug of azacitidine having the following structure: 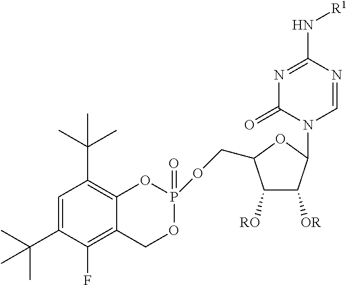
or a pharmaceutically acceptable salt thereof, where R and R¹ are independently H or CO₂(C₁-C₆ alkyl), is administered to the patient.

25. The method of claim 11, wherein 2′,3′,5′-triacetyl-5-azacitidine, or a pharmaceutically acceptable salt thereof, is administered to the patient.

26. The method of claim 11, wherein the HMA is decitabine, or a prodrug thereof, or a pharmaceutically acceptable salt of the foregoing.

27. (canceled)

28. A method of treating myelodysplastic syndrome (MDS) in a patient in need thereof, comprising administering to the patient a therapeutically effective amount of decitabine, or a prodrug thereof, or a pharmaceutically acceptable salt of the foregoing; and a therapeutically effective amount of alvocidib, or a prodrug thereof, or a pharmaceutically acceptable salt of the foregoing, wherein:
the decitabine, or a prodrug thereof, or a pharmaceutically acceptable salt of the foregoing, is administered on days 1, 2, 3, 4 and 5 of a 28-day treatment cycle; and
the alvocidib, or a prodrug thereof, or a pharmaceutically acceptable salt of the foregoing, is administered on day 8 of the 28-day treatment cycle,
wherein MCL-1 dependence of the MDS increases with administration of the decitabine, or a prodrug thereof, or a pharmaceutically acceptable salt of the foregoing. and the MDS has MCL-1 dependence of greater than or equal to 40% upon administration of the alvocidib, or a prodrug thereof, or a pharmaceutically acceptable salt of the foregoing.

29. The method of claim 26, wherein decitabine, or a pharmaceutically acceptable salt thereof, is administered to the patient.

30-34. (canceled)

35. The method of claim 29, wherein the decitabine, or a prodrug thereof, or a pharmaceutically acceptable salt of the foregoing, is administered orally.

36. The method of claim 1, wherein alvocidib, or a pharmaceutically acceptable salt thereof, is administered to the patient.

37-39. (canceled)

40. The method of claim 36, wherein about 30 mg/m² alvocidib, or a pharmaceutically acceptable salt thereof, is administered to the subject by intravenous bolus of about 30 minutes in duration, and about 60 mg/m² alvocidib, or a pharmaceutically acceptable salt thereof, is administered to the subject by intravenous infusion of about 4 hours in duration.

41-43. (canceled)

44. The method of claim 1, wherein a prodrug of alvocidib, or a pharmaceutically acceptable salt thereof, is administered to the patient.

45. The method of claim 44, wherein the prodrug of alvocidib has the following structural formula: 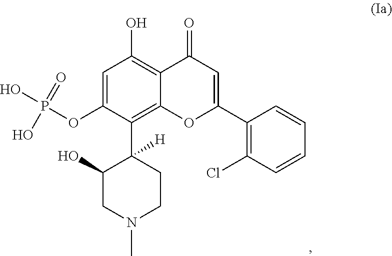 [SMILES: CN1CC[C@H](c2c(OP(=O)(O)O)cc(O)c3c(=O)cc(-c4ccccc4Cl)oc23)[C@H](O)C1]
or a zwitterionic form or pharmaceutically acceptable salt thereof.

46-73. (canceled)

74. The method of claim 1, wherein the patient has one or more mutations in RUNX1.

75. The method of claim 1, wherein the patient has one or more mutations in ASXL1.

76. The method of claim 1, wherein the patient has one or more mutations in RUNX1 and one or more mutations in ASXL1.

77. The method of claim 14, wherein the patient has one or more of the following: previously untreated MDS; received fewer than six cycles of treatment with a hypomethylating agent: de novo MDS; or secondary MDS.

78. The method of claim 28, wherein the patient has one or more of the following: previously untreated MDS; received fewer than six cycles of treatment with a hypomethylating agent: de novo MDS: or secondary MDS.

79-85. (canceled)
